(19) United States
(12) Patent Application Publication
(10) Pub. No.: US 2016/0069887 A1
(43) Pub. Date: Mar. 10, 2016

(54) BIOMARKERS FOR PROGNOSIS

(71) Applicant: ISIS INNOVATION LIMITED, Summertown, Oxford (GB)

(72) Inventor: Nicholas B. La Thangue, Bridge of Weir (GB)

(21) Appl. No.: 14/783,065

(22) PCT Filed: Apr. 8, 2014
(86) PCT No.: PCT/GB2014/051091

§ 371 Date: Oct. 7, 2015

(30) Foreign Application Priority Data
Apr. 8, 2013 (GB) 1306339.1

(51) Int. Cl. G01N 33/574; C12Q 1/68; A61K 45/06

(52) U.S. Cl. CPC G01N 33/57484; A61K 45/06; C12Q 1/6886; C12Q 2600/106

(57) ABSTRACT

The invention relates to biomarkers for determining the prognosis of cancer patients. By determining the level of the biomarker HR23B in the cell or patient, and optionally determining the levels of one or more of the biomarkers HDAC6, LC3 and HSP90, the invention provides a method for determining the susceptibility of a cell or a patient of interest to entering an autophagocytic state upon treatment with a drug. The invention also provides a method of determining susceptibility of a cell or patient to treatment with a drug. Entering an autophagocytic state is thought to be a tumour cell survival mechanism, whereby the tumour cell avoids apoptosis. The methods of the invention may therefore be helpful for determining whether a patient should be treated and for determining the prognosis upon drug treatment.

### BACKGROUND

Histone deacetylase is a family of enzymes that control the acetylation of chromatin (1). An increasingly large group of proteins connected with different aspects of normal and tumour cell biology are known to be influenced by acetylation (2-4). As a consequence, the HDAC family has attracted considerable attention as a therapeutic target (2, 3, 5). Indeed, inhibition of HDAC activity is strongly antiproliferative on tumour cells in vitro, and delays tumour growth in xenograft models (5-7). Accordingly, HDAC inhibitors are now a class of anti-cancer drug (1, 2, 16). An extensive number of clinical trials with HDAC inhibitors in a variety of malignancies are underway, and two HDAC inhibitors, SAHA/Vorinostat and FK228/Romidepsin, have to date been approved for treating a human malignancy, namely cutaneous T cell lymphoma (CTCL; (1, 8-10)). However, identifying other malignancies and disease types that are likely to respond favourably to HDAC inhibitors has been hampered, principally because knowledge of the key pathways through which HDAC inhibitors affect tumour cell growth remains limited (9, 11).

At a mechanistic level, inhibition of HDAC activity has profound anti-proliferative effects, and HDAC inhibitors have different outcomes on cells including induction of apoptosis, cell cycle arrest, senescence, differentiation and more recently autophagy (2, 5, 6, 20). However, the precise mechanisms through which HDAC inhibitors give rise to different biological consequences remains to be determined.

In previous studies, a genome-wide loss-of-function screen has been used to identify genes that impact on the sensitivity of tumour cells to HDAC inhibitors (12). It was reasoned that genes identified in this way would not only provide important information on key pathways and thereby illuminate mechanisms that are affected by HDAC inhibitors, but also identify potential biomarkers that inform on the tumour response to HDAC inhibitor-based therapies (11, 12). Through this analysis, HR23B was identified as a protein that influences the response and sensitivity of tumour cells to HDAC inhibitors (12, and see WO2007/110623, Isis Innovation Limited). Thus, HR23B is a useful biomarker for response to HDAC inhibitors.

HR23B functions in at least two pathways; nucleotide excision repair (NER) and protein targeting to the proteasome (13-15). Further studies suggested that the ability of HR23B to engage in proteasomal shuttling underpins its role as a determinant of HDAC inhibitor sensitivity, and it is consistent with this idea that aberrant proteasome activity occurs in tumour cells treated with HDAC inhibitors, which is likely to be an important mechanism in prompting apoptosis (12).

The potential utility of HR23B as a predictive biomarker has been evaluated in the clinical setting by studying its expression in biopsies taken from a group of patients suffering from CTCL that had been treated with Vorinostat (8, 16, 17). The analysis indicated that there was a good coincidence between HR23B expression and therapeutic response to treatment (16), suggesting that HR23B could provide a useful predictive biomarker for identifying CTCL that responds favourably to HDAC inhibitors.

However, treating tumour cells with HDAC inhibitors can result in a number of different outcomes, which includes apoptosis, senescence, autophagy and cell cycle arrest (2, 18-20). There is increasing evidence that tumour cells evade cell death through autophagy (34-36). For example, autophagy is initiated by chemotherapy and radiation, where it is believed to represent a tumour cell survival mechanism against stressful agents that induce apoptosis (37, 38). The fact that therapy-induced autophagy provides a survival advantage is supported from studies where pharmacological inhibition of autophagy enhanced the therapeutic effects of drugs in tumour regression models (39-41). Consequently, autophagy has emerged as a potential tumour cell survival mechanism and in turn a possible therapeutic target in cancer, since agents which inhibit autophagy would be expected to improve therapeutic outcomes when combined with conventional chemotherapeutics.

Given the variability in cellular response, and the potential utility of HR23B as a response-specific biomarker, it is interesting to evaluate whether there is a role for HR23B in the diverse cellular outcomes of HDAC inhibitor treatment. With this objective in mind, the present invention explores the mechanistic role of HR23B in regulating the biological consequence of HDAC inhibitor treatment.

### SUMMARY OF THE INVENTION

The invention provides a method for determining the susceptibility of a cell or a patient of interest to entering an autophagocytic state upon treatment with a drug, comprising determining the level of HR23B in the cell or patient.

Also provided is a method for determining the susceptibility of a cell or a patient of interest to treatment with a drug comprising determining the level of HR23B in combination with the level of one or more of HDAC6, LC3 and HSP90 in the cell or patient.

Also provided is a method for determining the susceptibility of a cell or a patient of interest to treatment with a drug comprising determining the level of HSP90 in the cell or patient.

HR23B is provided in combination with one or more of HDAC6, LC3 and HSP90 for use as biomarkers for determining the susceptibility of a cell or a patient of interest to treatment with a drug.

HSP90 is provided for use as a biomarker for determining the susceptibility of a cell or a patient of interest to treatment with a drug.

There is provided a kit for determining the level of HR23B in combination with the level of one or more of HDAC6, HSP90 and LC3 in a cell or patient of interest, wherein the kit comprises an anti-HR23B antibody and one or more of an anti-HDAC6 antibody, an anti-HSP90 antibody and an anti-LC3 antibody, optionally in combination with a label for visualising the antibodies.

The invention further provides a method of treating a disease or other condition with a drug, which comprises administering the drug to a cell or patient which has been identified as not having a low level of HR23B.

Similarly, the invention provides a method of treating a disease or other condition with a drug, which comprises administering the drug to a cell or patient who has been identified as having a high level of HR23B in combination with a low level of one or more of HDAC6, HSP90 and LC3 or as not having a low level of HR23B in combination with a high level of one or more of HDAC6, HSP90 and LC3.

Likewise, there is provided a drug for use in treating a disease or other condition in a cell or patient of interest who has been identified as having a high level of HR23B in combination with a low level of one or more of HDAC6, HSP90 and LC3 or as not having a low level of HR23B in combination with a high level of one or more of HDAC6, HSP90 and LC3.

Similarly, there is provided a method of treating a patient having a disease or other condition, wherein the disease or other condition has been determined to have a level of HR23B in combination with a level of one or more of HDAC6, HSP90 and LC3 that indicates that the disease is susceptible to treatment with a drug, wherein the treating comprises administering the drug to the patient.

Also provided is a method of treating a patient having a disease or other condition, wherein the disease or other condition has been determined not to have a low level of HR23B, wherein the treating comprises administering the drug to the patient.

Also provided is a method of increasing the susceptibility of a cell or patient of interest to treatment with a drug comprising increasing the level of HR23B and/or decreasing the level of HDAC6 and/or inactivating or decreasing the level of HSP90 and/or decreasing the level of LC3 in the cell or patient.

Likewise, there is provided a method of treating a disease or other condition with a drug, which comprises administering the drug to a cell or patient wherein the treating comprises administering the drug in combination with an HR23B-increasing agent and/or an HDAC6-decreasing agent and/or an HSP90 inhibitor and/or an LC3-decreasing agent, either simultaneously, separately or sequentially.

The invention provides a drug for use in treating a disease or other condition in a cell or patient of interest wherein the treating comprises administering the drugin combination with an HR23B-increasing agent and/or an HDAC6-decreasing agent and/or an HSP90 inhibitor and/or an LC3-decreasing agent, either simultaneously, separately or sequentially.

Similarly, there is provided an HR23B-increasing agent for use in treating a disease or other condition in a cell or patient of interest, wherein the treating comprises administering the HR23B-increasing agent simultaneously, separately or sequentially with a drug and optionally with an HDAC6-decreasing agent and/or with an HSP90 inhibitor and/or with an LC3-decreasing agent.

Similarly, there is provided an HDAC6-decreasing agent for use in treating a disease or other condition in a cell of patient of interest, wherein the treating comprises administering the HDAC6-decreasing agent simultaneously, separately or sequentially with a drug and optionally with an HR23B-increasing agent and/or an HSP90 inhibitor and/or an LC3-decreasing agent.

Similarly, there is provided an HSP90 inhibitor for use in treating a disease or other condition in a cell or patient of interest, wherein the treating comprises administering the HSP90 inhibitor simultaneously, separately or sequentially with a drug and optionally with an HR23B-increasing agent and/or an HDAC6-decreasing agent and/or an LC3-decreasing agent.

Similarly, there is provided an LC3-decreasing agent for use in treating a disease or other condition in a cell or patient of interest, wherein the treating comprises administering the LC3-decreasing agent simultaneously, separately or sequentially with a drug and optionally with an HR23B-increasing agent and/or an HDAC6-decreasing agent and/or an HSP90 inhibitor.

A product is provided comprising a drug in combination with one, two, three or all four of i) an HR23B-increasing agent, ii) an HDAC6-decreasing agent, iii) an HSP90 inhibitor, and iv) an LC3-decreasing agent in the treatment of a disease of other condition in a cell or patient of interest, wherein the drug in combination with one, two, three or all four of i) an HR23B-increasing agent, ii) an HDAC6-decreasing agent, iii) an HSP90 inhibitor, and iv) a LC3-decreasing agent are for simultaneous, separate or sequential administration

Also provided is a method to optimise the dosage of a drug comprising administering the drug to a cell or patient of interest, monitoring whether an apoptotic state or an autophagy state ensues, and administering a further dose of drug which has been adjusted if necessary, or alternatively not administering a further dose of the drug.

### DETAILED DESCRIPTION OF THE INVENTION

HR23B has surprisingly been found to be an autophagy marker. Autophagy is connected with poor prognosis in disease. Accordingly, the invention provides the use of HR23B as an autophagy marker. More specifically, the invention provides a method for determining the susceptibility of a cell or a patient of interest to entering an autophagocytic state, for example upon treatment with a drug, comprising determining the level of HR23B in the cell or patient. If the cell or patient of interest is determined to have a low level of HR23B, the cell or patient of interest is determined to be susceptible to entering an autophagocytic state. HR23B has the amino acid sequence shown in SEQ ID NO:16.

Accordingly, the invention also provides a method for determining the susceptibility of a cell or a patient of interest to treatment with a drug comprising determining the level of HR23B in the cell or patient, wherein the method further comprises determining that the cell or patient has a low level of HR23B and is therefore not susceptible to treatment with the drug.

Autophagy is becoming increasingly recognised as a mechanism that cancer cells use to survive under adverse conditions, for example, chemotherapy (Kroemer, G., Marino, G. & Levine, B. Autophagy and the integrated stress response. Mol Cell 40, 280-293 (2010)). As mentioned above, the present invention has surprisingly found that a low level of HR23B is prognostic for an autophagocytic response. LC3 is a well known marker for autophagy, with a high level of LC3 being prognostic for an autophagocytic response (42). It is reasoned therefore that tumours exhibiting different levels of HR23B and LC3 may differ in their clinical history and prognosis, reflecting the detrimental influence that surviving autophagocytic tumour cells might have on therapeutic response and clinical outcome. LC3 has the amino acid sequence shown in SEQ ID NO:17.

Accordingly, the invention provides a method for determining the susceptibility of a cell or a patient of interest to entering an autophagocytic state, for example upon treatment with a drug, comprising determining the levels of HR23B and a further autophagy marker, such as LC3, in the cell or patient. If the cell or patient of interest is determined to have a low level of HR23B and a high level of LC3, the cell or patient of interest is determined to be susceptible to entering an autophagocytic state. Conversely, if the cell or patient of interest is determined to have a high level of HR23B and a low level of LC3, the cell or patient of interest is determined to be susceptible to entering an apoptotic state.

As mentioned above, entering an autophagocytic state upon treatment with a drug or radiation is thought to be a tumour cell survival mechanism, whereby the tumour cell avoids apoptosis. Thus, determining the susceptibility of a cell or a patient of interest to entering an autophagocytic state, for example upon treatment with a drug, is helpful for determining whether a patient should be treated with a drug and for determining the cell or patient's prognosis upon drug treatment.

Accordingly, the invention provides a method of determining the susceptibility of a cell or patient of interest to treatment with a drug comprising determining the levels of HR23B and LC3 in the cell or patient. The method may further comprise determining that the cell or patient has a low level of HR23B and a high level of LC3 and is therefore not susceptible to treatment with the drug. Similarly, the method may further comprise administering the drug to a patient who has been found not to have a low level of HR23B and a high level of LC3.

Conversely, a high level of HR23B in combination with a low level of LC3 has surprisingly been found to be prognostic for an apoptotic response. Thus if a patient of interest is determined to have a high level of HR23B in combination with a low level of LC3, they are likely to respond well to treatment. Thus, the method may further comprise determining that the cell or patient has a high level of HR23B and a low level of LC3 and is therefore susceptible to treatment with the drug. Similarly, the method may further comprise administering the drug to a patient who has been found to have a high level of HR23B and a low level of LC3.

The drug for use in the invention may be any drug whose mechanism of action is apoptosis, for example, anti-cancer drugs, such as HDAC inhibitors or other chemotherapeutic agents. A preferred drug is an HDAC inhibitor.

Significantly, the present invention demonstrates that HR23B impacts on the outcome of HDAC inhibitor treatment by regulating the switch between apoptosis and autophagy. Within the cell-types and systems studied, it appears that high levels of HR23B render cells sensitive to HDAC inhibitor-induced apoptosis.

The results indicate that HR23B impacts on two documented effects of HDAC inhibitors, namely apoptosis and autophagy. Thus, HDAC inhibitors cause apoptosis in cells expressing high levels of HR23B, whereas in cells with low level expression, HDAC inhibitor treatment was frequently associated with autophagy. The mechanism responsible for regulating the outcome of treatment involves the interplay between HR23B and HDAC6, and specifically the ability of HDAC6 to counter-balance the pro-apoptotic effect of HR23B. This is achieved through an interaction between HDAC6 and HR23B, which down-regulates HR23B, reducing the level of ubiquitinated substrates targeted to the proteasome and ultimately desensitising cells to apoptosis.

HR23B targets the ubiquitin proteasome system (UPS), where it shuttles ubiquitinated cargo that are destined for subsequent proteasomal degradation (13, 14). Proteasomal degradation represents one of the most important pathways for regulating proteostasis (42), and one which is well documented as an anti-cancer drug target (43). Its integration with other levels of protein turnover, for example autophagosomal degradation, is also becoming increasingly evident (42, 44-47). The results presented herein suggest that the ability of HR23B to target the proteasome leads to aberrant proteasome activity which in turn sensitises tumour cells to apoptosis.

Accordingly, the invention provides the use of HR23B as a biomarker to determine whether a cell or patient of interest is a suitable candidate for treatment with an HDAC inhibitor, wherein a low level of HR23B indicates that the cell or the cells in the patient being treated will likely enter an autophagocytic state following treatment. Thus, looking at the levels of HR23B alone allows a prediction to be made as to whether a patient may be a likely responder to HDAC inhibitor treatment or whether the patient's cells may enter an autophagocytic state following treatment. Similarly, the invention provides a method for determining the susceptibility of a cell or a patient of interest to treatment with a HDAC inhibitor comprising determining the level of HR23B in the cell or patient, wherein the method further comprises determining that the cell or patient has a low level of HR23B and is therefore not susceptible to treatment with the HDAC inhibitor.

In addition, however, HDAC6 has now been identified as a negative regulator of HR23B, which thereby allows HDAC6 to impinge on proteasomal activity. Since HR23B sensitises tumour cells to apoptosis (12, 16), by virtue of its ability to downregulate HR23B, HDAC6 acts to counter-balance the pro-apoptotic activity of HR23B. It is consistent with this idea that HDAC6-deficient cells exhibit higher levels of HR23B, which coincides with an increased level of apoptosis and reduced susceptibility to autophagy and the ability of HDAC6 to down-regulate HR23B. Thus, the balance between HR23B and HDAC6 is important in dictating the effect of HDAC inhibitors on tumour cells.

These results therefore suggest that the interplay between HR23B and HDAC6 is important in regulating the biological outcome of drug treatment, for example, HDAC inhibitor treatment. Thus, the present invention has found that whether or not a cell or a patient is susceptible to treatment with a drug, for example with an HDAC inhibitor, can more accurately be determined by looking not only at the level of HR23B, but also at the level of HDAC6. Specifically, it has been found that cells or patients having a high level of HR23B and a low level of HDAC6 are suitable candidates for treatment with an HDAC inhibitor because the cells will enter an apoptotic state following treatment. In contrast, cells or patients having a low level of HR23B and a high level of HDAC6 are not suitable candidates for treatment with an HDAC inhibitor because the cells will enter an autophagocytic state following treatment. Entering an autophagocytic state is undesirable in situations where the treatment requires the cells to be killed, because an autophagocytic state means that the cell's survival mechanism has been induced, and generally means that the cells will be resistant to other cell-killing therapies, such as chemotherapy.

The inventors have also surprisingly found that a high level of HR23B does not always correlate with the cell entering an apoptotic state upon treatment with an HDAC inhibitor. Specifically, whilst cells having a high level of HR23B and a low level of HDAC6 will enter an apoptotic state when treated with an HDAC inhibitor, cells having a high level of HR23B and a high level of HDAC6 may either enter an apoptotic state or an autophagocytic state when treated with an HDAC inhibitor. Consequently, treating a patient having a high level of HR23B and a high level of HDAC6 with an HDAC inhibitor may make the disease or other condition worse, because it may lead to cells going into survival mode, thus making them even more difficult to kill. Thus, although using a high level of the HR23B biomarker alone does increase the likelihood that the cell or patient of interest will respond to HDAC inhibitor treatment, there are some cells or patients that have high levels of HR23B that will not be suitable candidates for HDAC inhibitor treatment. These findings are illustrated by Table 2 below:

TABLE 2

| Level of | Level of | Susceptible to treatment |
|---|---|---|
| HR23B | HDAC6 | with HDAC inhibitor? |
| High | Low | Yes →apoptosis |
| High | High | Unpredictable |
| Low | High | No → autophagy |
| Low | Low | Unpredictable |

Looking at the levels of HR23B in combination with the levels of HDAC6 therefore increases the accuracy of the prediction as it enables the skilled person to determine whether an apoptotic or autophagocytic state will arise as a result of HDAC inhibitor treatment and thereby permits the skilled person to assess whether or not to treat the cell or patient of interest with the HDAC inhibitor.

Accordingly, the present invention provides an improved method for assessing the susceptibility of a cell or patient of interest to treatment with a drug, for example an HDAC inhibitor. The improvement arises from the ability to look at HR23B in combination with one or more other biomarkers for autophagy and/or apoptosis, rather than just HR23B. In particular, the improvement arises from determining the level of the HR23B biomarker in combination with the level of one, two or all three of the LC3 biomarker, the HDAC6 biomarker and the HSP90 biomarker in the cell or patient. Preferred combinations include HR23B and HDAC6; HR23B and HSP90; HR23B and LC3; and HR23B, HDAC6 and HSP90. By assessing the levels of these biomarkers, the invention advantageously identifies new subclasses of patients which may be treated using a drug, for example an HDAC inhibitor. The present invention has important implications for personalised medicine approaches to drug treatment, in particular for HDAC inhibitor treatment.

Accordingly, in some embodiments, the invention provides a method for determining the susceptibility of a cell or a patient of interest to entering an autophagocytic state, for example upon treatment with a drug, comprising determining the levels of HR23B in combination with determining the levels of one or more of LC3, HDAC6 and HSP90 (for example, the levels of LC3 and HDAC6, LC3 and HSP90, HDAC6 and HSP90 or all of LC3, HDAC6 and HSP90) in the cell or patient. In some embodiments, the method is for determining the susceptibility of a cell or a patient of interest to entering an autophagocytic state in response to treatment with chemotherapy or radiation.

Accordingly, the invention provides a method for determining the susceptibility of a cell or a patient of interest to treatment with a drug, for example an HDAC inhibitor, comprising determining the levels of HR23B and HDAC6 in the cell or patient. The invention also provides the use of HR23B and HDAC6 in combination as biomarkers for determining the susceptibility of a cell or a patient of interest to treatment with a drug, for example an HDAC inhibitor. Similarly, the invention provides HR23B and HDAC6 in combination for use as biomarkers for determining the susceptibility of a cell or a patient of interest to treatment with a drug, for example an HDAC inhibitor. The method or use may further comprise determining whether a cell or patient of interest is suitable or is not suitable for treatment with a drug, for example an HDAC inhibitor.

Preferably, a cell or patient which is susceptible to treatment with a drug, for example an HDAC inhibitor, has a high level of HR23B and a low level of HDAC6. Accordingly, the method or use may further comprise administering the drug to the cell or patient of interest if the cell or patient has been found to have a high level of HR23B and a low level of HDAC6.

Advantageously, the invention also makes it possible to determine whether a cell or patient should not be treated with a drug, in particular in the event that obtaining an autophagy response is undesirable. Autophagy is generally undesirable when the drug is used for cancer treatment because a cell in autophagy has often adapted to survive chemotherapy. Preferably, a cell or patient which is not susceptible to treatment has a low level of HR23B and a high level of HDAC6.

Accordingly, the method or use may further comprise administering the drug to the cell or patient of interest if the cell or patient has been found not to have a low level of HR23B and a high level of HDAC6. Alternatively, the method may further comprise not administering the drug to the cell or patient of interest if the cell or patient has been found to have a low level of HR23B and a high level of HDAC6.

In some embodiments, the method comprises administering the drug to a cell or patient which has been found to have a high level of HR23B and a high level of HDAC6 or to a cell or patient which has been found to have a low level of HR23B and a low level of HDAC6. Although such a cell or patient is not identified as a good candidate for drug treatment according to the invention, neither is it identified as a bad candidate and so it may be desirable to treat the cell or patient with the drug in case an apoptotic response does ensue.

Where the invention relates to a cell or patient that “has been found [not] to have . . . ” a biomarker at a certain level, the cell or patient has preferably been found to have that level of biomarker using a method, use or kit according to the present invention.

The inventors have also surprisingly found that heat shock protein 90 (HSP90) is also involved in regulating the levels of HR23B. In exploring the mechanism through which HDAC6 downregulates HR23B, a series of protein networks have been identified that interact with HDAC6 and which potentially could be subject to control by HDAC6. Networks involving tubulins and chaperones were evident, as were others connected with DNA repair and cell metabolism. The inventors have observed that HSP90 is a key effector in the HDAC6-dependent down-regulation of HR23B, because the ability of HDAC6 to down-regulate HR23B was overcome by treating cells with the HSP90 inhibitor 17-AAG.

HDAC6 has been found to downregulate HR23B via HSP90. Significantly, the ability of HDAC6 to down-regulate HR23B occurred independently of its deacetylase activity, and a proteomic analysis of the HDAC6 interactome identified HSP90 as a key effector in the HDAC6-mediated down-regulation of HR23B.

It is noteworthy that the HSP90 network is responsible for regulating HR23B, because HDAC6 is established to control HSP90 chaperone activity, through a deacetylation event that regulates client protein and chaperone binding (30, 48). Through its HR23B binding BUZ domain and HSP90 activating catalytic domain, it is envisaged that HDAC6 is thus able to couple proteasome and chaperone activity, in addition to a major influence on HR23B levels (FIG. 5h). The inventors have elucidated that the mechanism which underpins HDAC6 and its effect on HR23B involves the interplay with the HSP90 (via HDAC6 BUZ domain interacting with HR23B UbL domain).

Accordingly, the invention also provides a method for determining the susceptibility of a cell or a patient of interest to treatment with an HDAC inhibitor comprising determining the level of HSP90 in the cell or patient. Generally, a low level of HSP90 will be indicative of a patient that would be susceptible to treatment with a drug, for example an HDAC inhibitor, because HDAC6 cannot have its negative regulatory effect on HR23B in the absence of HSP90. Thus, in some embodiments, the levels of HSP90 can be used alone as a biomarker. In some embodiments, the method further comprises administering the HDAC inhibitor to a cell or patient which has been found to have a low level of HSP90. However, it is advantageous to determine the level of at least HR23B in addition to HSP90. In other embodiments, the methods of determining the susceptibility of a cell or patient to treatment with a drug, for example an HDAC inhibitor, comprise determining the levels of HR23B, HDAC6 and HSP90 or HR23B, HDAC6, LC3 and HSP90 in the cell or patient.

When a cell or patient is determined by the invention to not be susceptible to treatment with a drug, such as an HDAC inhibitor, the invention may further comprise treating the cell or patient with a more aggressive apoptotic regime, for example, a chemotherapy regime with cytotoxic drugs such as irinotecan, oxaliplatin and 5-FU.

The invention may also be used to determine the susceptibility of a particular disease or other condition to treatment with a drug, such as an HDAC inhibitor. For example, if a particular disease or other condition is found to be associated with a particular biomarker profile as described herein, a cell or patient of interest with that particular disease or other condition can then be determined to be susceptible or not to treatment with the drug, such as an HDAC inhibitor, according to the techniques described herein. Thus, the cell whose level of the HR23B biomarker is being determined, optionally in combination with the level of one or more (e.g. 1, 2 or 3) of the HDAC6 and/or HSP90 and/or LC3 biomarker may be a cell from a particular disease or other condition and thereby representative of a particular disease or other condition. Identifying that the cell has a particular biomarker profile allows a correlation to be made that the disease also has that biomarker profile. Thus, it is not always necessary to test the biomarker profile in each and every patient being diagnosed. Instead, if a patient is diagnosed as having a particular disease, a treatment regimen can be designed according to the prior knowledge of the biomarker profile of that particular disease.

As a non-limiting example, the invention provides a method for determining the susceptibility of a particular disease or other condition to treatment with a drug, comprising determining the biomarker levels associated with the disease. For example, in accordance with the invention, a low level of HR23B is prognostic for an autophagy response and so is indicative of a lack of susceptibility to treatment with the drug. Similarly, a low level of HR23B combined with a high level of LC3 and/or HDAC6 and/or HSP90 is indicative of a lack of susceptibility to treatment with the drug. Conversely, a high level of HR23B combined with a low level of LC3 and/or HDAC6 and/or HSP90 is indicative of susceptibility to treatment with the drug. Thus the method may further comprise treating a patient with the drug wherein the patient has been diagnosed as having a disease or other condition that is susceptible to treatment with the drug, for example, according to a method as described herein. More specifically, the invention provides a method of treating a patient having a disease or other condition, wherein the disease or other condition has been determined to have a biomarker profile as described herein that is susceptible to treatment with a drug, for example an HDAC inhibitor, wherein the treating comprises administering the drug to the patient. Conversely, the method may further comprise treating a patient with the drug wherein the patient has been diagnosed as having a disease or other condition which, for example using a method as described herein, has not been found not to be susceptible to treatment with the drug.

HR23B and/or LC3 and/or HDAC6 and/or HSP90 are referred to herein collectively as the “one or more biomarkers”.

Where the invention refers to levels of the one or more biomarkers “in a patient”, this is to be understood as being the levels of the one or more biomarkers in cells of interest from the patient. The cells of interest will generally be the cells associated with the disease or condition being treated by the drug, for example, with the HDAC inhibitor. For example, where the patient of interest has cancer, the cells of interest are preferably the cancerous cells from the patient. An example of a suitable cell is a tumour cell, for example, a Circulating Tumour Cell (CTC). Similarly, the biomarker profile of the disease may be assessed by taking a sample of the cells associated with the disease. The determining step is preferably carried out in vitro. Thus, the method is preferably an in vitro method. The cell or cells of interest may be obtained from the patient by any suitable method, for example, by a tissue biopsy, in particular a tumour biopsy, or from a sample of body fluid such as saliva, blood, serum, plasma, lymph or urine, or from a stool sample. The skilled person will understand which sample is appropriate depending on the type of disease or other condition being treated. For example, if the patient of interest is being tested for susceptibility to treatment of breast cancer, it would be appropriate to determine the levels of the one or more biomarkers in breast cancer cells from said patient.

The invention may also be used to test the susceptibility of healthy cells in a patient to treatment with a drug, for example an HDAC inhibitor. For example, it may be desirable to determine whether a patient's healthy cells are likely to be affected by treatment with a drug as a side-effect of the desirable drug treatment, such as anti-cancer treatment.

Preferably the patient/cell is a human patient/cell. However, it is also envisioned that the invention may be used with non-human patients/cell. In some embodiments, the patient/cell is an animal patient/cell, for example, a mammal (for example, a non-human mammal). Examples of mammals that may be used in the various aspects of the present invention include primates (for example, non-human primates), rodents (for example, mice or rats), rabbits, a laboratory test animal, a domestic animal (for example a cat or dog), and a farm animal (for example a cow, pig or sheep). In some embodiments, the patient/cell is a bird (for example, a chicken).

Levels of the one or more biomarkers may be determined by any suitable method. In some embodiments, a ligand for the biomarker whose levels are being determined, such as an antibody against the biomarker, is used. Thus, determining the level of the one or more biomarkers may comprise the steps of a) contacting a ligand of the one or more biomarkers, such as an antibody against the one or more biomarkers, with a biological sample under conditions suitable for the formation of ligand-protein complex; and b) detecting said complex.

For example, in some embodiments, anti-HR23B and/or anti-LC3 and/or anti-HDAC6 and/or anti-HSP90 antibodies are used to bind to the one or more biomarkers within the cell. Suitable antibodies include anti-HR23B (CellSignalling), anti-HDAC6 (Millipore), anti-HSP90α/β (Santa Cruz) and anti-LC3 (microtubule-associated protein1 light chain 3B, for example, clone 5F10, mouse monoclonal, IgG1, Epitope: N-terminus of LC3-B, company: nanoTools, order number: 0231-100/LC3-5F10). In some embodiments, the anti-HR23B antibody targets an epitope on HR23B within the SPTA rich domain, which runs from amino acid residue 73 to 193 of SEQ ID NO: 16. In some embodiments, the anti-HR23B antibody targets an epitope on HR23B selected from DPEETVKALKEKIE (amino acids 18-31 of SEQ ID NO:16; SEQ ID NO:18); PAPASAAKQEKPAE (amino acids 116-129 of SEQ ID NO:16; SEQ ID NO:19); or VATSPTATDSTSGD (amino acids 136-149 of SEQ ID NO:16; SEQ ID NO:20).

As used herein, the term “antibody” refers to full length antibody molecules as well as to fragments thereof, such as Fab, F(ab′)2 and Fv fragments, which are capable of binding to the antigenic determinant in question. Preferably, the antibody binds specifically to the biomarker whose levels are being determined. The term “binds specifically” means that the antibody has substantially greater affinity for its target polypeptide than its affinity for other related polypeptides, and preferably do not cross-react with other proteins. By “substantially greater affinity” we mean that there is a measurable increase in the affinity for the target biomarker as compared with the affinity for other related polypeptide. Preferably, the affinity is at least 1.5-fold, 2-fold, 5-fold 10-fold, 100-fold, 10³-fold, 10⁴-fold, 10⁵-fold, 10⁶-fold or greater for the target polypeptide. Preferably, the antibodies bind to the target biomarker with high affinity, preferably with a dissociation constant of 10⁻⁴M or less, preferably 10⁻⁷M or less, most preferably 10⁻⁹ M or less; subnanomolar affinity (0.9, 0.8, 0.7, 0.6, 0.5, 0.4, 0.3, 0.2, 0.1 nM or even less) is preferred.

Preferred methods for determining the levels of the one or more biomarkers are visual techniques, in particular those which are easy to visualise. For example, immunostaining, such as immunohistochemistry, is a preferred method.

The antibodies used for determining the levels of the one or more biomarkers may be labelled with any suitable marker, for example, a visual marker such as a fluorescence marker (for example fluorescein or rhodamine), or a dye. In some embodiments, the antibodies may be conjugated to an enzyme, such as peroxidase (e.g. horse-radish peroxidase) or alkaline phosphatase, which can catalyse a colour-producing reaction. In some embodiments, the antibodies may be visualised by targeting with a secondary labelled antibody which contains a visual marker.

In some embodiments, the determining step is carried out on paraffin-embedded tissue. For example, in some embodiments, the cell (for example, from a patient sample) can be seeded onto a coverslip and be washed and fixed before being permeabilised. Following permeabilisation, the cell can then be incubated with a primary antibody against the biomarker of interest. The coverslips can then be washed and incubated with a corresponding secondary antibody labelled with an appropriate marker, such as a fluorescent marker. The coverslips may then be washed and mounted onto microscope slides to enable detection of the level of the biomarker. In some embodiments, the levels of the one or more biomarkers are determined in tissue culture, for example, using fluorescence markers. In some embodiments, the levels of the one or more biomarkers are determined by staining of a tissue biopsy, for example, using immunohistochemistry.

A further suitable method for determining the levels of the one or more biomarkers is mass spectrometry, for example, using the multiple reaction monitoring (MRM) technique (Guo, B. et al., Curr. Drug. Metab., 2012 November; 13(9):1226-43). Mass spectrometry using the MRM technique is a particularly useful method for determining the levels of the one or more biomarkers in serum.

A further suitable method for determining the levels of the one or more biomarkers is assessing the levels of a nucleic acid encoding the respective biomarkers in the cells (for example DNA, cDNA, and/or RNA, e.g. mRNA), for example, the ratios of the respective DNA and/or RNA (for example, mRNA) levels. In some embodiments, the nucleic acid being detected comprises the complement of the DNA sequence encoding the biomarker. Where the level of a nucleic acid is detected, it is preferably detected by the steps of contacting a tissue sample with a probe under stringent conditions that allow the formation of a hybrid complex between the nucleic acid and the probe; and detecting the formation of a complex.

In some embodiments, the nucleic acid being detected is detected with a nucleic acid probe. Such nucleic acid probes can be labelled to facilitate detection. Common labels include radioactive phosphates, biotin, fluorophores and enzymes.

Thus, in some embodiments, the method or use comprises determining the levels of the one or more biomarkers using a machine, for example, a microscope or a mass spectrometer. In some embodiments, the levels of the one or more biomarkers are determined using reverse transcription PCR or Western Blot. Generally, more than one cell will be present in a patient sample and so the method or use may be used to determine the level of the biomarker in the more than one cell.

It is not necessary to determine the absolute levels of the one or more biomarkers in order to practice the invention. Instead, it is required to determine whether the levels are high or low. This can be assessed at a crude level. It is standard in the art to assess whether a level is high or low. Generally, the level will be assessed using a total staining score (TSS), which will be dependent not only on the level of the marker in the sample (e.g. in the tumour sample), but also on the number of cells in the sample. The threshold level for diagnosis would depend on the particular disease and sample type under analysis. The cut-off level between a sample being “high” and normal or between a sample being “low” and normal is readily calculated by mathematical analysis of the data (typically a receiver operating characteristic (ROC) curve) which is common practice for a practitioner in the field (for example see Metz, “Basic principles of ROC analysis”, Seminars in Nuclear Medicine (1978) 8: 283-298 and Zweig and Campbell, “Receiver-operating characteristic (ROC) plots: A fundamental evaluation tool in clinical medicine”, Clinical Chemistry (1993) 39: 561-577). In some embodiments in which a ROC curve is used to determine whether a level is high or low, a level is high if it falls within the top 5%, 10%, 15%, 20%, 25%, 30% or 35% (preferably within the top 25%) of the cells and a level is low if it falls within the bottom 5%, 10%, 15%, 20%, 25%, 30% or 35% (preferably within the bottom 25%) of the cells.

In some embodiments, the levels are compared to a standard control to allow a determination to be made as to whether a level is high or low. An example of a suitable positive control is a cell which has been engineered to express a high level of the biomarker whose level is being determined (e.g. HR23B, HDAC6, HSP90 or LC3). If the test cell has a similar level of the biomarker as the control, for example, as assessed using the visual technique, then the test cell can be determined to have a high level of the biomarker. If the test cell does not have a similar level of the biomarker to the positive control, for example it has a much lower level, or the biomarker is absent, for example as assessed using a visual technique, then the test cell can be determined to have a low level of the biomarker. Thus, generally, a level that is similar to the positive control will indicate that the cell has a high level of the biomarker being assessed. By a “similar” level to a positive control is generally meant that the level of expression is the same as or is 1%, 5%, 15%, 20%, 30%, 40% or less lower than the positive control level, or is higher than the control level. In some embodiments, a negative control may also be used. An example of a suitable negative control is a cell which has been engineered so that is does not express the biomarker. In such cases, if the test cell has a similar level of the biomarker to the negative control, for example, as assessed using a visual technique, then the test cell can be determined to have a low level of the biomarker. The cell used as the positive or negative control is preferably from the same tissue type (such as the same tumour type) as the test cell and may in some embodiments be from a cell line. Preferably, prior to being engineered, the control is a close genetic match to the test cell.

In some embodiments, the control is the corresponding non-diseased tissue from which the diseased tissue is derived, preferably from the same patient, although this is not essential. Generally, a level that is significantly higher than the reference level in the non-diseased control will indicate that the level of the biomarker is high. Generally, a level that is significantly lower than the reference level in the non-disease control is indicative that the level of the biomarker is low. By “significant” is meant that the level of expression is more than 10%, 25%, 50%, 100%, 250%, 500%, 1000% or more, higher or lower, respectively, than the reference level.

In a further aspect of the invention, there is a provided a kit for determining the levels of the one or more biomarkers in a cell or patient of interest. In preferred embodiments, the kit may be used to determine the levels of both HR23B and HDAC6. In other embodiments, the kit may be used to determine the levels of HR23B and LC3. In other embodiments, the kit may be used to determine the levels of HR23B and HDAC6 and HSP90. In other embodiments, the kit may be used to determine the levels of HR23B, HDAC6, HSP90 and LC3. The kit may comprise all or only some of the agents required to carry out a method of determining the levels of the one or more biomarkers. Preferably, the agents required to determine the levels of the one or more biomarkers are agents as described herein. In some embodiments, the kit comprises an anti-HR23B antibody and/or an anti-HDAC6 antibody and/or an anti-HSP90 antibody and/or an anti-LC3 antibody. The kit may usefully also contain a label or labels for visualising the antibodies. The label or labels may be part of the antibodies or may, for example, be on a secondary antibody. Preferred labels are fluorescent labels. In some embodiments, the labels on the anti-HR23B antibody and/or the anti-HDAC6 antibody and/or the anti-HSP90 antibody and/or the LC3 antibody are different. This permits the relative levels of the one or more biomarkers to be assessed in the same cell or in the same group of cells simultaneously. However, in some embodiments, the labels may be the same and for instance, the levels of the different biomarkers may then be determined in different cells. In some embodiments, the anti-HR23B antibody and/or the anti-HDAC6 antibody and/or the anti-HSP90 antibody and/or the anti-LC3 antibody are each in separate containers. In some embodiments, the kit comprises one or more probes for determining the levels of the one or biomarkers at the nucleic acid level. In some embodiments, the kit comprises a combination of protein ligands (such as antibodies) and nucleic acid probes.

### Methods of Treatment

In a further aspect, the invention provides a method of treating a disease or other condition with a drug such as an HDAC inhibitor, which comprises administering the drug to a cell or patient who has been identified as being susceptible to treatment with the drug. Examples of patients identified by the invention as being susceptible to treatment with a drug are patients having a high level of HR23B in combination with a low level of one or more of HDAC6, HSP90 and LC3 or not having a low level of HR23B in combination with a high level of one or more of HDAC6, HSP90 and LC3. The levels of the one or more biomarkers may be determined using a method of the invention. Similarly, there is provided the use of a drug such as an HDAC inhibitor in the manufacture of a medicament for treating a patient who has been identified as being susceptible to treatment with the drug. Likewise, there is provided a drug such as an HDAC inhibitor for use in treating a patient who has been identified as being susceptible to treatment with the drug. Preferably, the cell or cells from the patient have a high level of HR23B and a low level of HDAC6. Preferably, the cell or cells from the patient do not have a low level of HR23B and a high level of HDAC6. In some embodiments, the cell or cells from the patient have a high level of HR23B and a low level of LC3. In some embodiments, the cell or cells from the patient do not have a low level of HR23B and a high level of LC3. In some embodiments, the cell or cells from the patient have a high level of HR23B and a low level of HSP90. In some embodiments, the cell or cells from the patient do not have a low level of HR23B and a high level of HSP90. In some embodiments, the cell or cells from the patient have a combination of these biomarker profiles.

In some embodiments, there is provided a drug, such as an HDAC inhibitor, for use in treating a disease or other condition, wherein the drug is administered to the cell or patient on the basis of a sample from the cell or patient having been determined to have a level of the one or more biomarkers which indicates that the cell or patient is susceptible to treatment with the drug.

In some embodiments, there is provided a drug, such as an HDAC inhibitor, for use in treating a disease or other condition, characterised in that the drug is administered to a cell or patient from whom a sample has been taken, and which sample has been determined to have a level of the one or more biomarkers which indicates that the cell or patient is susceptible to treatment with the drug.

In some embodiments, there is provided a drug, such as an HDAC inhibitor, for use in a method of treating a disease or other condition, wherein the method comprises administering the drug to a cell or patient who has been determined to have a level of the one or more biomarkers which indicates that the cell or patient is susceptible to treatment with the drug.

In some embodiments, there is provided a drug, such as an HDAC inhibitor, for use in a method of treating a disease or other condition, wherein the method comprises steps of: (a) determining the levels of the one or more biomarkers in the cell or patient; and (b) if the results of step (a) indicate that the cell or patient has a level of the one or more biomarkers which indicates that the cell or patient is susceptible to treatment with the drug, administering the drug to the cell or patient. Any suitable drug may be used in the present invention. Drugs whose mechanism of action is apoptosis are well known. Anti-cancer drugs are good examples of such drugs which may be used. For example, any suitable HDAC inhibitor may be used in the present invention. Examples of suitable HDAC inhibitors for use in the invention include superoylanilide hydroxamic acid (“SAHA”) (also known as Vorinostat or N-hydroxy-N′-phenyloctanediamide or C₁₄H₂₀N₂O₃, for example, marketed as Zolinza® by Merck), FK228/Romidepsin (for example, marketed as Istodax®, Celgene) and valproic acid. In some embodiments, the HDAC inhibitor is selected from N-hydroxy-N′-phenyl-octanediamide (Vorinostat), (1 S,4S,7Z,10S,16E,21R)-7-ethylidene-4,21-diisopropyl-2-oxa-12,13-dithia-5,8,20,23-tetrazabicyclo[8.7.6]tricos-16-ene-3,6,9,19,22-pentone (Romidepsin), (2E)-N-hydroxy-3-[4-({[2-(2-methyl-1H-indol-3-yl)ethyl]amino}methyl)phenyl]acrylamide (Panobinostat), 2-Propylpentanoic acid (Valproic acid), (2E)-3-[3-(anilinosulfonyl)phenyl]-N-hydroxyacrylamide (Belinostat), 4-(acetylamino)-N-(2-aminophenyl)benzamide (CI-994), ((E)-N-hydroxy-3-[4-[[2-hydroxyethyl-[2-(1H-indol-3-yl)ethyl]amino]methyl]phenyl]prop-2-enamide (Dacinostat), 3-(dimethylaminomethyl)-N-[2-[4-(hydroxycarbamoyl)phenoxy]ethyl]-1-benzofuran-2-carboxamide (PCI-24781), N-hydroxy-2-(4-(naphthalen-2-ylsulfonyl)piperazin-1-yl)pyrimidine-5-carboxamide (R306465), N-(2-aminophenyl)-4-[[(4-pyridin-3-ylpyrimidin-2-yl)amino]methyl]benzamide (Mocetinostat), pyridin-3-ylmethyl 4-(2-aminophenylcarbamoyl)benzylcarbamate (Entinostat), 4-(dimethylamino)-N-[7-(hydroxyamino)-7-oxoheptyl]benzamide (M344), (E)-3-(2-butyl-1-(2-(diethylamino)ethyl)-1H-benzo[d]imidazol-5-yl)-N-hydroxyacrylamide (SB939), (E)-3-(1-((4-((dimethylamino)methyl)phenyl)sulfonyl)-1H-pyrrol-3-yl)-N-hydroxyacrylamide (Resminostat), {6-[(diethylamino)methyl]naphthalen-2-yl}methyl[4-(hydroxycarbamoyl)phenyl]carbamate (Givinostat), N-Hydroxy-2-[4-({[(1-methyl-1H-indol-3-yl)methyl]amino}methyl)-1-piperidinyl]-5-pyrimidinecarboxamide (Quisinostat), pivaloyloxymethyl butyrate (AN-9), N-(2-aminophenyl)-N′-phenyloctanediamide (BML-210), phenylbutyrate, 7-(4-(3-ethynylphenylamino)-7-methoxyquinazolin-6-yloxy)-N-hydroxyheptanamide (CUDC-101), (S)—N-hydroxy-4-(3-methyl-2-phenylbutanamido)benzamide (AR-42), 2-(6-(((6-fluoroquinolin-2-yl)methyl)amino)bicyclo[3.1.0]hexan-3-yl)-N-hydroxypyrimidine-5-carboxamide (CHR-3996), CHR-2845, 4SC-202, (E)-N1-(3-(dimethylamino)propyl)-N8-hydroxy-2-((naphthalen-1-yloxy)methyl)oct-2-enediamide (CG200745), 2-(diphenylamino)-N-(7-(hydroxyamino)-7-oxoheptyl)pyrimidine-5-carboxamide (ACY-1215), 4,4′-(7-hydroxy-8-methylchroman-3,4-diyl)diphenol (ME-344), and 1-Isothiocyanato-4-methylsulfinylbutane (sulphoraphane).

The patient being assessed using a method of the invention, or being treated with the drug, such as an HDAC inhibitor, may have any disease or other condition in which autophagy or HDACs play a role. Autophagy correlates with a poor prognosis in cancer and inflammatory diseases. Thus, the disease or other condition is preferably cancer or an inflammatory disease, but may also be an infectious disease, cardiovascular disease or a neurological disease.

In a preferred embodiment, the drug, such as an HDAC inhibitor, is used to treat cancer in the patient of interest. Similarly, the cells of interest are preferably cancer cells, for example, tumour cells. Examples of cancers/tumour cells that may be treated using the methods of the invention include cutaneous T-cell lymphoma (CTCL) and peripheral T-cell lymphoma (PTCL). In some embodiments, the patient has CTCL and has progressive, persistent or recurrent disease on or following two systemic therapies. Accordingly, in some embodiments, the drug, such as an HDAC inhibitor, is used to treat cutaneous manifestations in such a patient. Zolinza® has been licensed for use in such patients. In other embodiments, the patient has CTCL and has received at least one prior systemic therapy or has PTCL and has received at least one prior therapy. Istodax® is licensed for such uses:

In some embodiments, the disease or other condition is selected from acute myeloid leukaemia, advanced breast cancer, advanced malignant pleural mesothelioma, advanced prostate cancer, advanced stage non-small cell lung cancer, brain cancer, breast cancer, castration-resistant prostate cancer, cervical cancer, chronic lymphocytic leukemia, chronic myeloic leukaemia, colorectal cancer, cutaneous T cell lymphoma (CTCL), follicular lymphoma, gastrointestinal carcinoma, hepatocellular carcinoma, HER-2 positive metastatic breast cancer, Hodgkin lymphoma, lung cancer, lymphoid malignancies, lymphoma, mantle cell lymphoma, melanoma, meningioma, metastatic castration-resistant prostate cancer, metastatic colorectal cancer, metastatic prostate cancer, micropapillary ovarian tumors, myelodysplastic syndrome, myelofibrosis, myeloid malignancy, myeloma, non-small cell lung cancer, ovarian cancer, peripheral T cell lymphoma, platinum resistant epithelial ovarian cancer, progressive multiple myeloma, prostate cancer, recurrent glioblastoma multiforme, refractory colorectal cancer, refractory Hodgkin lymphoma, refractory leukaemia, refractory multiple myeloma, relapsed Hodgkin lymphoma, relapsed multiple myeloma, relapsed peripheral T-cell lymphoma, sarcoma, schwannoma, solid refractory tumour, solid tumour, T cell lymphoma, thymic malignancies, and treatment-resistant multiple myeloma.

In alternative embodiments, the disease or other condition is selected from polycythemia vera, essential thrombocythemia, myelofibrosis, acute myocardial infarction, spinal muscular atrophy, epilepsy, bipolar disorder, depression, absence seizures, tonic-clonic seizures (grand mal), complex partial seizures, juvenile myoclonic epilepsy, and myoclonus.

In some embodiments, the HDAC inhibitor is N-hydroxy-N′-phenyl-octanediamide (Vorinostat) and the disease or condition is cutaneous T cell lymphoma, acute myeloid leukaemia, follicular lymphoma, mantle cell lymphoma, solid tumour, advanced prostate cancer, recurrent glioblastoma multiforme, relapsed multiple myeloma, refractory multiple myeloma, myelodysplastic syndrome, lymphoid malignancies, gastrointestinal carcinoma, metastatic colorectal cancer, non-small cell lung cancer (NSCLC), advanced stage NSCLC, refractory colorectal cancer, or advanced breast cancer (Wagner et al (2010) Clin. Epigenet. 1:117-136).

In other embodiments, the HDAC inhibitor is (1S,4S,7Z,10S,16E,21R)-7-ethylidene-4,21-diisopropyl-2-oxa-12,13-dithia-5,8,20,23-tetrazabicyclo[8.7.6]tricos-16-ene-3,6,9,19,22-pentone (Romidepsin) and the disease or condition is cutaneous T cell lymphoma, relapsed peripheral T-cell lymphoma, metastatic castration-resistant prostate cancer, advanced colorectal cancer, or refractory multiple myeloma (Wagner et al (2010) Clin. Epigenet. 1:117-136).

In other embodiments, the HDAC inhibitor is (2E)-N-hydroxy-3-[4-({[2-(2-methyl-1H-indol-3-yl)ethyl]amino}methyl)phenyl]acrylamide (Panobinostat) and the disease or condition is cutaneous T cell lymphoma, relapsed Hodgkin lymphoma, refractory Hodgkin lymphoma, myelofibrosis, multiple myeloma, relapsed multiple myeloma, chronic myeloic leukaemia, castration-resistant prostate cancer or HER-2 positive metastatic breast cancer (Wagner et al (2010) Clin. Epigenet. 1:117-136).

In one embodiments, the HDAC inhibitor is (2E)-3-[3-(anilinosulfonyl)phenyl]-N-hydroxyacrylamide (Belinostat) and the disease or condition is lymphoma, peripheral T cell lymphoma, cutaneous T cell lymphoma, platinum resistant epithelial ovarian cancer, micropapillary ovarian tumors, advanced malignant pleural mesothelioma, solid tumors, or thymic malignancies (Wagner et al (2010) Clin. Epigenet. 1:117-136).

In other embodiments, the HDAC inhibitor is {6-[(diethylamino)methyl]naphthalen-2-yl}methyl[4-(hydroxycarbamoyl)phenyl]carbamate (Givinostat) and the disease or condition is relapsed or progressive multiple myeloma or refractory leukaemia (Wagner et al (2010) Clin. Epigenet. 1:117-136).

In other embodiments, the HDAC inhibitor is pyridin-3-ylmethyl 4-(2-aminophenylcarbamoyl)benzylcarbamate (Entinostat) and the disease or condition is a myeloid malignancy (Wagner et al (2010) Clin. Epigenet. 1:117-136).

In other embodiments, the HDAC inhibitor is 2-Propylpentanoic acid (Valproic acid) and the disease or condition is myelodysplastic syndrome, acute myeloid leukaemia, solid tumour, melanoma, cervical cancer, ovarian cancer, or spinal muscular atrophy (Wagner et al (2010) Clin. Epigenet. 1:117-136).

In other embodiments, the HDAC inhibitor is N-(2-aminophenyl)-4-[[(4-pyridin-3-ylpyrimidin-2-yl)amino]methyl]benzamide (Mocetinostat), and the disease or condition is follicular lymphoma, Hodgkin lymphoma, or acute myeloid leukaemia.

In other embodiments, the HDAC inhibitor is 3-(dimethylaminomethyl)-N-[2-[4-(hydroxycarbamoyl)phenoxy]ethyl]-1-benzofuran-2-carboxamide (PCI-24781) and the disease or condition is sarcoma or lymphoma.

In other embodiments, the HDAC inhibitor is pyridin-3-ylmethyl 4-(2-aminophenylcarbamoyl)benzylcarbamate (Entinostat) and the disease or condition is Hodgkin lymphoma, lung cancer or breast cancer.

In other embodiments, the HDAC inhibitor is (E)-3-(2-butyl-1-(2-(diethylamino)ethyl)-1H-benzo[d]imidazol-5-yl)-N-hydroxyacrylamide (SB939) and the disease or condition is recurrent or metastatic prostate cancer or myelofibrosis.

In other embodiments, the HDAC inhibitor is (E)-3-(1-((4-((dimethylamino)methyl)phenyl)sulfonyl)-1H-pyrrol-3-yl)-N-hydroxyacrylamide (Resminostat) and the disease or condition is Hodgkin lymphoma or hepatocellular carcinoma.

In other embodiments, the HDAC inhibitor is (S)—N-hydroxy-4-(3-methyl-2-phenylbutanamido)benzamide (AR-42) and the disease or condition is relapsed or treatment-resistant multiple myeloma, chronic lymphocytic leukaemia, lymphoma, meningioma, or schwannoma.

In other embodiments, the HDAC inhibitor is (E)-N1-(3-(dimethylamino)propyl)-N8-hydroxy-2-((naphthalen-1-yloxy)methyl)oct-2-enediamide (CG200745) and the disease or condition is a solid tumour.

In other embodiments, the HDAC inhibitor is 2-(diphenylamino)-N-(7-(hydroxyamino)-7-oxoheptyl)pyrimidine-5-carboxamide (ACY-1215) and the disease or condition is multiple myeloma.

In other embodiments, the HDAC inhibitor is 4,4′-(7-hydroxy-8-methylchroman-3,4-diyl)diphenol (ME-344) and the disease or condition is a solid refractory tumour.

The levels of the one or more biomarkers in the patient may be manipulated to make a particular cell or patient of interest more susceptible to treatment with a drug, such as an HDAC inhibitor. Accordingly, there is provided a method of increasing the susceptibility of a cell or patient of interest to treatment with a drug such as an HDAC inhibitor comprising increasing the level of HR23B and/or decreasing the level of HDAC6. Advantageously, inactivating HSP90 sensitizes cells to treatment with a drug such as an HDAC inhibitor because it prevents the negative regulation of HR23B by HDAC6. Thus, in some embodiments, the method of increasing the susceptibility of a cell or patient of interest to treatment with a drug such as an HDAC inhibitor comprises inactivating or decreasing the levels of HSP90. In some embodiments, the method of increasing the susceptibility of a cell or patient of interest to treatment with a drug such as an HDAC inhibitor comprises decreasing the levels of LC3.

The terms “increasing” the level of HR23B and/or “decreasing” the level of HDAC6 and/or HSP90 and/or LC3 encompass increasing or decreasing, respectively, the absolute levels of these proteins. However, it also encompasses increasing or decreasing, respectively, the biologically available levels of the one or more biomarker proteins without affecting their absolute levels. For example, “increasing” the level of HR23B encompasses increasing the level of HR23B by decreasing the sequestration of the HR23B protein and making greater amounts biologically available, without affecting the absolute amount of the HR23B protein. In particular, by blocking the interaction of HDAC6 with HR23B, HDAC6 will no longer have an inhibitory effect on HR23B at the protein level, thus making more HR23B biologically available.

Accordingly, the drug such as an HDAC inhibitor may be administered in combination with an agent which increases the level of HR23B in the cell or patient of interest and/or with an agent which decreases the level of HDAC6 in the cell or patient of interest and/or with an agent which inactivates or decreases the level of HSP90 in the cell or patient of interest and/or with an agent which decreases the level of LC3 in the cell or patient of interest. The term “HR23B-increasing agent” is used herein to refer to an agent which increases the level of HR23B in the cell or patient of interest. The term “HDAC6-decreasing agent” is used herein to refer to an agent which decreases the level of HDAC6 in the cell or patient of interest. The term “HSP90 inhibitor” is used herein to refer to an agent which inhibits or which decreases the level of HSP90 in the cell or patient of interest. The term “LC3-decreasing agent” is used herein to refer to an agent which decreases the level of LC3 in the cell or patient of interest. For example, the drug may be administered in combination with an HR23B-increasing agent and an HDAC6-decreasing agent; or the drug may be administered in combination with an HR23B-increasing agent; or the drug may be administered in combination with an HR23B-increasing agent, an HDAC6-decreasing agent and an HSP90 inhibitor; or the drug may be administered in combination with an HR23B-increasing agent and an HSP90 inhibitor; or the drug may be administered in combination with an HSP90 inhibitor; or the drug may be administered in combination with an HR23B-increasing agent and an LC3-decreasing agent; or the drug may be administered in combination with a HR23B-increasing agent, an HDAC6-decreasing agent and an HSP90 inhibitor; or the drug may be administered in combination with a HR23B-increasing agent, an HDAC6-decreasing agent, an HSP90 inhibitor and an LC3-decreasing agent; or any combination of the above examples.

Any suitable agent may be used to increase the level of HR23B in the cell or patient of interest. In some embodiments, the HR23B-increasing agent may be an agent which increases the levels of biologically available HR23B. Such agents do not necessarily lead to an increase in the absolute level of the HR23B protein. Indeed, the inventors have found that HDAC6 exerts its negative regulation of HR23B at the protein level. Consequently, in some embodiments, the HR23B-increasing agent inhibits the interaction between HDAC6 and HR23B. The inventors have elucidated that the mechanism which underpins HDAC6 and its effect on HR23B involves the interplay with the HSP90 (via HDAC6 BUZ domain interacting with HR23B UbL domain). Thus, in some embodiments, the HR23B-increasing agent targets the BUZ domain on HDAC6 (see Hard, R. L. et al. Biochemistry, 2010, 49, 10737-10746 for a description of the BUZ domain). Without wishing to be bound by theory, in targeting this domain, it is envisaged that the agent disrupts the interaction of HDAC6 with HR23B and/or HSP90. In some embodiments, the HR23B-increasing agent targets the UbL domain on HR23B (13, 14). Without wishing to be bound by theory, in targeting this domain, it is envisaged that the agent disrupts the interaction of HR23B with HDAC6 and/or HSP90. Examples of suitable agents for inhibiting this interaction are anti-HDAC6 antibodies, anti-HR23B antibodies, and specifically antibodies which target the HDAC6 Buz domain or the HR23B Ubl domain.

In some embodiments, the HR23B-increasing agent is HR23B itself. The HR23B protein and encoding nucleic acid are well known in the art and their sequences are provided in Genbank sequence NM_{—}002874.4. In some embodiments, the HR23B-increasing agent is a polypeptide comprising at least 80%, 85%, 90%, 95%, 98%, 99% sequence identity to the HR23B sequence. In other embodiments, the HR23B-increasing agent may be a nucleic acid encoding the HR23B protein. The nucleic acid may comprise, for example, a DNA sequence, a cDNA sequence or an RNA sequence encoding HR23B. In some embodiments, the nucleic acid is contained in a vector, such as a DNA plasmid or a viral vector. In some embodiments, the nucleic acid is administered in isolated form. The skilled person would be able to administer the protein or nucleic acid to the cell or patient of interest using techniques well known in the art. In some embodiments, the HR23B-increasing agent is an agent which upregulates HR23B expression, for example, by increasing the rate of transcription or translation of HR23B.

Any suitable agent may be used to decrease the level of HDAC6 in the cell or patient of interest. In some embodiments, the HDAC6-decreasing agent is an antisense RNA, an siRNA, or an shRNA which targets and knocks down the HDAC6 mRNA. The skilled person would understand how to design suitable antisense RNA, siRNA, shRNA using standard techniques in the art. An example of a suitable HDAC6 siRNA is 00349900 (Dharmacon). A non-limiting example of a suitable HDAC shRNA comprises siRNA sequences generated against the amino terminus of HDAC6 at nucleotides 211-231 and 217-237. In some embodiments, synthetic siRNAs are introduced directly into the cells. In some embodiments, a nucleic acid producing the HDAC6-decreasing agent, such as an siRNA or shRNA, is present on a vector. In some embodiments, the vector ensures long term production of the nucleic acid in the cell and may in some embodiments be a viral vector. In some embodiments, a gene therapy agent may be used to decrease the level of HDAC6 at the DNA level, for example to knock out HDAC6 expression. For example, in some embodiments, the agent may be a site-specific endonuclease, such as a TALEN, which specifically recognises the DNA encoding the HDAC6 protein and degrades at least some of the sequence, thereby either preventing the protein being expressed or rendering inactive any protein which is expressed. The cDNA sequence for HDAC6 is shown in SEQ ID NO:10. Accordingly, in some embodiments, the HDAC6-decreasing agent may be a gene therapy agent, such as an endonuclease, which targets SEQ ID NO:10. In some embodiments, the HDAC-decreasing agent is tubastatin. In some embodiments, the HDAC6-decreasing agent decreases the level of HDAC6 at the protein level. For example, the HDAC6-decreasing agent may be an agent, such as a targeted protease, which targets and degrades HDAC6. In some embodiments, the HDAC6-decreasing agent may be a small molecule. The amino acid sequence of HDAC6 is provided in SEQ ID NO:11.

Any suitable agent may be used to inactivate HSP90. Examples of suitable HSP90 inhibitors include small molecules, antibodies, RNAi, siRNA, shRNA and antisense RNAs which target HSP90. HSP90 has two forms, alpha and beta, and the present invention envisages inhibiting either or both of these with the same or different HSP90 inhibitor. The cDNA and amino acid sequence of HSP90-alpha are provided in SEQ ID NO:12 and 13, respectively. The cDNA and amino acid sequence of HSP90-beta are provided in SEQ ID NO:14 and 15, respectively. Preferred inhibitors are small molecules. Suitable HSP90 inhibitors include 17-AAG (33), geldanamycin, radicicol and AUY922 (Novartis).

Any suitable agent may be used to decrease the level of LC3. Examples of suitable LC3-decreasing agents include small molecules, antibodies, RNAi, siRNA, shRNA and antisense RNAs which target LC3. Suitable agents include drugs which inhibit autophagy, for example, hydroxychloroquine or bafilomycin. Pharmacological inhibitors of autophagy can be broadly classified as early- or late-stage inhibitors of the pathway. Early-stage inhibitors include 3-methyadenine, wortmannin, and LY294002, which target the class III PI3K (Vps34) and interfere with its recruitment to the membranes. Late-stage inhibitors include the antimalarial drugs CQ, hydroxychloroquine (HCQ), bafilomycin A1, and monensin. Bafilomycin A1 is a specific inhibitor of vacuolar-ATPase (66), and monensin and CQ/HCQ are lysosomotropic drugs that prevent the acidification of lysosomes, whose digestive hydrolases depend on low pH. Autophagosomes and lysosomes move along microtubules, and microtubule-disrupting agents (taxanes, nocodazole, colchicine, and vinca alkaloids) inhibit fusion of autophagosomes to lysosomes. Other inhibitors of autophagy that block autophagosome degradation include the tricyclic antidepressant drug clomipramine and the anti-schistome agent lucanthone (67, 68).

Sequences of the biomarkers are provided herein. However, the invention is not limited to the biomarkers having these specific sequences and extends to variants of these sequences. For example, it is well understood that different individuals possess allelic variants of proteins which differ from the corresponding protein in another individual by way of one or more mutations, for example, one or more deletions, insertions, substitutions (for example conservative substitutions), etc. In some embodiments, the variant biomarker protein may have a nucleic acid or protein sequence with 60% or more (for example 70%, 75%, 80%, 85%, 90%, 95%, 98%, 99% or more) sequence identity to the nucleic acid or protein sequence for the respective biomarker provided herein. In some embodiments, the variant biomarker may be a domain mutant which lacks one or more domains of the biomarker. Thus, in some embodiments, the biomarker is a biomarker as described herein or an allelic variant thereof. In some embodiments, the biomarker protein is a homologue of the biomarker in a different species, for example in a non-human animal, and the invention is being carried out in a non-human animal. In some embodiments, the biomarker is a fragment of a biomarker as described herein or a fragment of a variant thereof.

The HR23B-increasing agent and/or the HDAC6-decreasing agent and/or the HSP90 inhibitor and/or the LC3-decreasing agent may be in the form of a pharmaceutical composition. For example, they may be present in combination with a pharmaceutically acceptable carrier and/or excipient and may also be present in combination with a drug such as an HDAC inhibitor.

The drug such as an HDAC inhibitor may be administered in combination with an HR23B-increasing agent and/or with an HDACC6-decreasing agent and/or with an HSP90 inhibitor and/or with an LC3-decreasing agent, either simultaneously, separately or sequentially. It is envisaged that administering the HR23B-increasing agent and/or the HDAC6-decreasing agent and/or the HSP90 inhibitor and/or the LC3-decreasing agent before administering the drug is an advantageous order for administration because it will make the cell competent for apoptosis when treated with the drug. Accordingly, in preferred embodiments, the HR23B-increasing agent and/or the HDAC6-decreasing agent and/or the HSP90 inhibitor and/or the LC3-decreasing agent are administered before the drug. However, embodiments in which the drug is administered before the HR23B-increasing agent and/or the HDAC6-decreasing agent and/or the HSP90 inhibitor and/or the LC3-decreasing agent are also encompassed by the invention.

Accordingly, there is provided a method of treating a disease or condition which comprises administering a drug such as an HDAC inhibitor simultaneously, separately or sequentially with an HR23B-increasing agent and/or with an HDAC6-decreasing agent and/or with an HSP90 inhibitor and/or with an LC3-decreasing agent.

Similarly, there is provided a drug such as an HDAC inhibitor for use in treating a disease or condition, wherein the drug is administered simultaneously, separately or sequentially with an HR23B-increasing agent and/or with an HDAC6-decreasing agent and/or with an HSP90 inhibitor and/or with an LC3-decreasing agent.

There is also provided an HR23B-increasing agent for use in treating a disease or condition in the patient of interest, wherein the HR23B-increasing agent is administered simultaneously, separately or sequentially with a drug such as an HDAC inhibitor and optionally with an HDAC6-decreasing agent and/or with an HSP90 inhibitor and/or with an LC3-decreasing agent.

For example, there is provided an HR23B-increasing agent for use in treating a disease or condition in the patient of interest, wherein the HR23B-increasing agent is administered simultaneously, separately or sequentially with an HDAC inhibitor, and wherein the patient of interest has low levels of HDAC6. Prior to treatment, this patient would not be a good candidate for treatment with an HDAC inhibitor, because of the low levels of HR23B combined with the low levels of HDAC6, which makes it unpredictable whether the patient's cells will enter an apoptotic or autophagy response, following treatment with an HDAC inhibitor. However, by manipulating the levels of HR23B, such that they are increased, this method preferably results in a patient having high levels of HR23B coupled with low levels of HDAC6, which greatly increases the likelihood that the patient's cells will enter the apoptotic pathway following treatment with an HDAC inhibitor, thereby making the patient a good candidate for treatment with an HDAC inhibitor. Preferably, this embodiment results in a patient having a high level of HR23B and a low level of HDAC6, i.e. it results in a patient who is a good candidate for treatment with an HDAC inhibitor.

Similarly, there is provided an HR23B-increasing agent for use in treating a disease or other condition in the patient of interest, wherein the HR23B-increasing agent is administered simultaneously, separately or sequentially with a drug such as an HDAC inhibitor, and wherein the patient of interest has high levels of HDAC6. Prior to treatment, this patient would not be a good candidate for treatment with a drug such as an HDAC inhibitor, because of the low levels of HR23B combined with the high levels of HDAC6 leading to an autophagy response. However, by manipulating the levels of HR23B, such that they are increased, this method increases the likelihood of the patient's cells entering the apoptotic pathway following treatment with the HDAC inhibitor.

Likewise, there is provided an HDAC6-decreasing agent for use in treating a disease or other condition in the patient of interest, wherein the HDAC6-decreasing agent is administered simultaneously, separately or sequentially with a drug such as an HDAC inhibitor, and wherein the patient of interest has high levels of HR23B. There is also provided an HDAC6-decreasing agent for use in treating a disease or other condition in the patient of interest, wherein the HDAC6-decreasing agent is administered simultaneously, separately or sequentially with a drug such as an HDAC inhibitor and optionally an HR23B-increasing agent and/or an HSP90 inhibitor.

Similarly, there is provided an HSP90 inhibitor for use in treating a disease or other condition in the patient of interest, wherein the HSP90 inhibitor is administered simultaneously, separately or sequentially with a drug such as an HDAC inhibitor and optionally with an HR23B-increasing agent and/or an HDAC6-decreasing agent. There is also provided an HSP90 inhibitor for use in treating a disease or other condition in the patient of interest, wherein the HSP90 inhibitor is administered simultaneously, separately or sequentially with a drug such as an HDAC inhibitor, and wherein the patient of interest has high levels of HR23B.

Similarly, there is provided an HSP90 inhibitor for use in treating a disease or other condition in the patient of interest, wherein the HSP90 inhibitor is administered simultaneously, separately or sequentially with a drug such as an HDAC inhibitor, and wherein the patient of interest has high levels of HR23B.

There is also provided a product comprising a drug such as an HDAC inhibitor and one or both of i) an HR23B-increasing agent and ii) an HDAC6-decreasing agent as a combined preparation for simultaneous, separate or sequential use in treating a disease or other condition in a cell or patient of interest. Similarly, the invention also provides a product comprising a drug such as an HDAC inhibitor in combination with one, two or all three of i) an HR23B-increasing agent, ii) an HDAC6-decreasing agent, and iii) an HSP90 inhibitor in the treatment of a disease of other condition in a cell or patient of interest, wherein the drug in combination with one, two or all three of i) an HR23B-increasing agent, ii) an HDAC6-decreasing agent, and iii) an HSP90 inhibitor are for simultaneous, separate or sequential administration.

The drug such as an HDAC inhibitor and optionally one, two or all three of i) an HR23B-increasing agent, ii) an HDAC6-decreasing agent, and iii) an HSP90 inhibitor may, in some embodiments, be administered in combination with one or more other anti-cancer agents. For example, the drug and optionally one, two or all three of i) an HR23B-increasing agent, ii) an HDAC6-decreasing agent, and iii) an HSP90 inhibitor may be administered in combination with surgery, radiotherapy, chemotherapy, targeted therapy, immunotherapy, hormonal therapy or angiogenesis therapy.

Preferably, the drug such as an HDAC inhibitor is not administered in combination with an autophagy-inducing drug such as an MTOR inhibitor.

In some embodiments, the drug such as an HDAC inhibitor and optionally one, two, three or all four of i) an HR23B-increasing agent, ii) an HDAC6-decreasing agent, iii) an HSP90 inhibitor; and iv) an LC3-decreasing agent is/are administered in combination with one or more (e.g. 2, 3, 4 or more) of the following agents: Carboplatin, Paclitaxel, Bortezomib, Pegylated-Liposomal-Doxorubicin, Idarubicin, Cytarabine, Lenalidomide, Dexamethasone, Decitabine, Rituximab, Ifosphamide, Etoposide, 5-Fluorouracil, Leucovorin, Oxaliplatin, Tamoxifen, Gemcitabine, Cistplainum, Erlotinib, Docetaxel, Melphalan, Imatinib, Docetaxel, Trastuzumab, 5-Azacytidine, Epirubicin, FEC100, Karenitecin, Dacarbazin, or Ifα.

Further, there is provided a kit comprising a drug such as an HDAC inhibitor and one, two, three or all four of i) an HR23B-increasing agent, ii) an HDAC6-decreasing agent, iii) an HSP90 inhibitor; and iv) an LC3-decreasing agent. In some embodiments, the drug such as an HDAC inhibitor and, if present, the HR23B-increasing agent, the HDAC6-decreasing agent, the HSP90 inhibitor; and the LC3-decreasing agent are in combination with a pharmaceutically acceptable carrier and/or excipient. In some embodiments, the drug such as an HDAC inhibitor and one or more, e.g. 2, 3 or 4 of the HR23B-increasing agent, the HDAC6-decreasing agent, the HSP90 inhibitor; and the LC3-decreasing agent are present as a combined preparation for simultaneous, separate or sequential use in treating the disease or other condition.

The skilled person will understand at what dose to administer the drug such as an HDAC inhibitor to treat the disease or other condition, what route of administration is appropriate and whether there are any other contraindications based upon common general knowledge, for example, on the information contained in the product information for the commercially available licensed drugs and on the clinical trial data that are available. In some embodiments, the drug is used at a dose of under 100 mg/kg, for example, about 0.1 to about 50 mg/kg, about 0.5 to about 30 mg/kg or about 1 to about 10 mg/kg. Similarly, the skilled person will be able to determine the dose of the one, two, three or all four of i) an HR23B-increasing agent, ii) an HDAC6-decreasing agent, iii) an HSP90 inhibitor; and iv) a LC3-decreasing agent. In some embodiments, the drug and optionally one, two, three or all four of i) an HR23B-increasing agent, ii) an HDAC6-decreasing agent, iii) an HSP90 inhibitor, and iv) a LC3-decreasing agent are administered hourly, daily, weekly, monthly or bi-monthly, as appropriate.

However, the optimal drug dose may not be exactly the same for all patients. By monitoring the autophagy or apoptotic state of a cell or patient of interest in response to drugtreatment, it is possible to optimise the dose of the drug to be given. For example, the invention provides a method to optimise the drug dosage comprising administering the drug to a cell or patient of interest, monitoring whether an apoptotic state or an autophagy state ensues, and administering a further dose of drug which has been adjusted if necessary or alternatively not administering a further dose of the drug. The step of monitoring whether an apoptotic state or an autophagy state ensues preferably comprises determining the levels of one or more biomarkers of apoptosis and/or autophagy. For example, the method may comprise monitoring the level of the autophagy marker LC3. It is desirable to have low levels of the autophagy marker LC3 when treating a cell or patient, because low levels of LC3 are prognostic for an apoptotic response rather than an autophagy response. However, if the levels of LC3 being to increase when the cell or patient is treated with the drug, then the dosage is inappropriate and it may be appropriate to adjust the dosage or cease treatment. Similarly, the method may further comprise monitoring the level of the autophagy marker HR23B. It is desirable to have high levels of the autophagy marker HR23B when treating a cell or patient, because low levels of HR23B are prognostic for an autophagy response rather than an apoptotic response. However, if the levels of HR23B begin to decrease when the cell or patient is treated with the drug, then the dosage is inappropriate and it may be appropriate to adjust the dosage or cease treatment. The methods may further comprise monitoring the levels of one or more markers of apoptosis, such as keratin 18, caspase cleavage or PARP cleavage. In some embodiments, the levels of the autophagy or apoptotis markers are measured in the blood, for example in the serum of the patient of interest. For example, caspase cleavage and PARP cleavage are markers which appear in the blood on cancer cell death.

The invention also encompasses treating cells in vitro, as described herein, for example, for experimental purposes.

### General

The term “comprising” encompasses “including” as well as “consisting” e.g. a composition “comprising” X may consist exclusively of X or may include something additional e.g. X+Y.

The terms “a” and “an” encompass both singular and plural embodiments, for example, one and more than one (for example, two, three, four or five).

Reference to “one or more” encompasses each of 1, 2, 3, 4, 5, 10, 25, 50, 100, 150, 200, 500, 1000, or more, as appropriate.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Methods of determining sequence identity are well known in the art. References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30). A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in Smith & Waterman (1981) Adv. Appl. Math. 2: 482-489.

The practice of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al., Molecular Cloning, A Laboratory Manual (2001) Cold Harbor-Laboratory Press, Cold Spring Harbor, N.Y. or Ausubel et al., Current protocols in molecular biology (1990) John Wiley and Sons, N.Y.

Unless defined otherwise, all technical and scientific terms used herein have the same meaning as commonly understood by one of ordinary skill in the art to which this invention belongs.

The invention will now be described further with reference to the following figures and examples.

### BRIEF DESCRIPTION OF THE FIGURES

FIG. 1: Autophagy and HR23B in cells treated with HDAC inhibitors

a) HUT78(i) and MYLA(ii) (CTCL), and HCT116(iii) and HCT15(iv) (CRC) cells were each treated with SAHA at the indicated (μM) concentrations, extracts prepared at 18 hr and immunoblotted with anti-HR23B, anti-LC3, anti-LAMP1, anti-cathepsin D or anti-actin antibody as indicated; note that the appearance of the lower form (LC3-II) and increased overall LC3 level is indicative of autophagy. Visualisation of autophagosomes in U2OS cells by immunostaining with anti-LC3 (in red) in SAHA (10 and 20 μM) treated cells is also shown (v). Counter-stain with DAPI was superimposed for each image.

b) U2OS cells were treated with SAHA at the indicated (μM) concentration together with 3-methyladenine (3MA; 10 mM) for 18 hr, when cell extracts were immunoblotted with the indicated antibodies.

c) i) U2OS cells stably expressing inducible HR23B were either untreated or treated with SAHA as indicated (in μM for 18 hr) under non-induced (−) or induced (+) doxycycline treatment conditions (1 μg/ml; induction for 72 hr treatment). Cells were harvested and immunoblotted with anti-Flag (for ectopic HR23B), anti-LC3 or actin as indicated.

ii) U2OS stable cells were treated as described in (i) with SAHA (10 μM) for the indicated times (in hours), and the level of apoptosis analysed by immunoblotting for poly(ADP-ribose) (PAR) polymerase “PARP” (cleaved and uncleaved) under non-induced (−) or induced (+) doxycycline treatment conditions as indicated. The level of ectopic HR23B and actin is shown.

iii) U2OS cells were treated with HR23B or control (non-targeting “NT”) siRNA (50 nM for 72 hr) in the absence (−) or presence of SAHA (5 and 10 μM) and the level of PARP (cleaved and uncleaved) measured. The level of HR23B and actin is shown.

d) i) Autophagosomes in U2OS cells visualised with anti-LC3 (red) stably expressing inducible HR23B (visualised with anti-Flag; green) after treatment with SAHA (10 μM for 18 hr) in the absence (−) or presence (+) of doxycycline. (−) shows the LC3 staining autophagosomes (red) under SAHA treatment, and (+) shows superimposed LC3 (red) and HR23B (green) images.

ii) The level of HR23B and LC3 was analysed by immunoblotting with anti-Flag, PARP (cleaved form is shown) and LC3 as indicated (ii), and quantitation of cells with autophagosomes after treatment with SAHA is presented in (iii), in the absence (−) or presence (+) of doxycycline, which was performed in triplicate (error bars indicate SEM).

e) i) U2OS cells stably expressing HR23B were grown in duplicate in the absence (−) or presence (+) of doxycycline together with SAHA (indicated concentration in μM) and, after 9 days, the number of viable cell colonies was assessed by crystal violet staining. The untreated control cells are shown for comparison.

ii) Graphical representation of the data in (i).

FIG. 2: HR23B impacts on the ubiquitin proteasome system

a) i) U2OS cells stably expressing inducible ectopic Flag-HR23B were transfected with His₆-ubiquitin (His-Ub; 2 μg), then treated with doxycycline (+ and −, 1 μg/ml) for 24 hr following an additional 20 h treatment with SAHA (5 μM) and/or 4 hr treatment with MG132 (20 μM) as indicated. Cell lysates and Ni²⁺ pull-down eluates were analyzed by immunoblotting with anti-His as described. The input HR23B is shown underneath, together with actin levels.

ii) Quantitation of the ubiquitin signal.

b) i) U2OS cells were transfected with control (non-targeting “NT”) or HR23B siRNA (50 nM) for 24 hr, then transiently transfected with His₆-ubiquitin (His-Ub; 2 μg) for 24 hr, following an additional 20 hr treatment with SAHA (5 μM) and/or 4 hr treatment with MG132 (20 μM) as indicated. Ni²⁺ pull-down eluates were analyzed by immunoblotting with anti-His. The input HR23B level is shown underneath, together with actin levels.

ii) Quantitation of the ubiquitin signal.

c) i) U2OS cells expressing inducible ectopic Flag-HR23B were transiently transfected with GFP^{u} or GFP (100 ng), then treated with or without doxycycline (+ or −, 1 μg/ml) for 24 hr as indicated. Cell lysates were analyzed as described. Levels of GFP, HR23B and actin are shown.

ii) Quantitation of the GFP signal.

d) i) U2OS cells were transfected with control (NT) or HR23B siRNA (50 nM) for 24 hr, then transiently transfected with GFP or GFP^{u} (100 ng) for 48 hr as indicated. Cell lysates were analyzed as described above. Levels of GFP, HR23B and actin are shown.

ii) Quantitation of the GFP signal.

FIG. 3: Interplay between HDAC6 and HR23B

a) Extracts prepared from U2OS cells were immunoprecipitated with anti-HR23B or anti-HDAC6, followed by immunoblotting with the indicated antibody. The level of HR23B and HDAC6 is shown in the input (In), together with the control (C) immunoprecipitation. HR23B and HDAC6 are indicated.

b) Extracts prepared from WT or HDAC6 stable shRNA expressing (knock down “KD”) A549 cells were immunoblotted with the indicated antibodies.

c) U2OS cells were transfected with control (NT) or HDAC6 siRNA (50 nM) for 24 hr, then transfected with GFP or GFP^{u} for 48 hr as indicated. Cell lysates were analyzed as described. Levels of GFP, HR23B, HDAC6 and actin are shown. Quantification of the GFP signal is shown underneath.

d) U2OS cells stably expressing inducible ectopic Flag-HDAC6 were treated with doxycycline (+ and −; 1 μg/ml) for 72 hr, and transiently transfected with GFP^{u} or GFP (100 ng) for 48 hr as indicated. Cell lysates were analyzed as described. Quantification of the GFP signal is shown underneath.

e) Either WT or HDAC6 stable shRNA expressing (KD) A549 cells were treated with SAHA for 24 hr as indicated and thereafter the level of sub-G1 (apoptotic) cells measured by flow cytometry (i and ii; n=3, error bars indicate SEM). Extracts were prepared and immunoblotted with antibodies for LAMP1, cathepsin D and LC3 to assess the level of apoptosis and autophagy, in addition to HDAC6, HR23B and actin (iii).

f and g) Lysates from WT and HDAC6 stable shRNA expressing (KD) cells (f), or WT cells treated with HDAC6, HR23B or NT siRNA for 48 hr (g). Cell lysates were analysed by immunoblotting with anti-Ub for endogenous ubiquitination (f and g). Input levels of HDAC6, HR23B and actin are shown underneath. Quantification of the ubiquitin signal is presented in the graph.

h and i) U2OS cells were transfected with control (NT) or HDAC6 siRNA for 48 hr, and then immunostained in h) for LC3 (red) and HDAC6 (green), and in i) for cleaved PARP (red) and HDAC6 (green). Merged images are shown for each (h and i). Quantification performed by counting 500 cells under NT or HDAC6 siRNA treatment conditions is shown underneath.

FIG. 4: Interaction between HR23B and HDAC6

a) U2OS cells were transfected with Flag-HDAC6, ΔBUZ, 1-503, ΔN or BUZ (i). Cells were harvested and the HDAC6 mutants were immunoprecipitated with anti-Flag antibody 24 hr after transfection and subsequently immunoblotted with anti-Flag or anti-HR23B antibody (ii and iii); note that HR23B is the endogenous protein. Input HDAC6 levels are shown together with actin as a loading control. The C-terminal BUZ region of HDAC6 was required for the interaction with HR23B (note that ΔBUZ was expressed at higher levels compared to WT HDAC6, which could account for the weak coimmunoprecipitated HR23B with ΔBUZ).

b) U2OS cells were co-transfected with Flag-HDAC6 and HR23B (WT) and its different mutants lacking the UbA domains and the UbL domain (i). Cells were harvested and HDAC6 was immunoprecipitated with anti-Flag antibody 24 hr after transfection and subsequently immunoblotted with anti-Flag (Flag IB) or anti-Myc antibody (for HR23B). The input level of HDAC6 and HR23B mutants is shown. Only WT HR23B and ΔUbA were co-immunoprecipitated with HDAC6 (ii). Note that the enhanced level of ΔUbA compared to WT HR23B in the immunoprecipitation reflects the increased expression level of the input ΔUbA.

c) Immunoblot (i) showing endogenous HR23B levels upon induction (Dox+treatment) of Flag-HDAC6 in the stable inducible cell line over the indicated time course.

(ii) shows endogenous HR23B (red) in the Flag-HDAC6 (green) stable cell line in the same conditions, as indicated at 72 and 96 hr post induction (Dox+). DAPI and the merged image is shown.

d and e) WT and HDAC6 stable shRNA expressing (KD) cells were transfected with HDAC6 (WT) or ΔBUZ (d; 1 and 2 μg), and BUZ (e; 1, 2 and 3 μg) for 72 h as indicated. Cell lysates were analysed by immunoblotting as described with the indicated antibodies. Note that ectopic full-length (WT) HDAC6 is poorly expressed compared to BUZ in transfected cells and is indicated by the arrow ( [symbol] ) in FIG. 4d. Endogenous HR23B levels decrease upon expressing WT HDAC6 or BUZ domain, reduced by over 50% by the BUZ domain alone (FIG. 4e; 3 μg BUZ transfection treatment compared to—KD treatment).

FIG. 5: Analysis of HDAC6 interacting proteins

a) The stable Flag-HDAC6 cell line was treated with (+) or without (−) doxyclycline, and the autophagy inhibitor 3MA (5 and 10 mM), or SAHA (10 μM) as indicated.

b) Both control cells expressing the vector only (TRE) and stable cells expressing Flag-HDAC6 were induced with doxycyclin (+; 1 μM) for 60 hr or left uninduced. Extracts were immunoprecipitated with anti-Flag antibody and subsequently immunoblotted with anti-Flag antibody. Input levels are shown together with actin as loading control.

c) Co-immunoprecipitation of the Flag-HDAC6 and the empty vector TRE control were stained with a colloidal blue. Dots indicate specific bands in the HDAC6 co-immunoprecipitate which were excised and analysed by mass spectrometry. The prominent band around 150 kDa (indicated by the bracket) is the immune-enriched Flag-HDAC6. Table 1 provides further details of the interacting proteins.

d) In silico analysis of HDAC6 interacting proteins found by mass spectrometry are shown for which experimental data have been annotated in STRING (50). The proteins were clustered by the Markov Cluster Algorithm (MCL) and every colour corresponds to one protein cluster. The diameter of the circle represents the betweenness centrality (calculated by the program Gephi) and is a measure of the importance of the protein in the analysed network.

e) The stable Flag-HDAC6 cell line was treated with (+) or without (−) doxycycline. Extracts were immunoprecipitated with anti-Flag followed by immunoblotting with anti-HSP90.

f) The stable Flag-HDAC6 cell line was treated with (+) or without (−) doxycycline. Extracts were immunoprecipitated with anti-Flag followed by immunoblotting with anti-Ku80/XRCC5 or anti-GRP78/HSPA5.

g) The immunoblot shows the endogenous levels of HR23B upon induction (Dox treatment) of the Flag-HDAC6 stable inducible cell line. Two days after the induction, the HDAC6 specific inhibitor tubastatin (10 μM) and/or the HSP90 specific inhibitor 17-AAG (0.1 or 1 μM) were added and the cells were harvested 2 days later. Acetylated tubulin provides a control for the tubastatin treatment. Extracts were immunoblotted with anti-HSP90, anti-HR23B, anti-tubulin, anti-acetylated tubulin, anti-caspase 3 (cleaved) and anti-actin, as indicated.

h) Model for interplay between HR23B and HDAC6 in regulating the cellular response to HDAC inhibitors. It is envisaged that the level of HR23B and HDAC6 impacts on the response to HDAC inhibitors, reflected as apoptotic or autophagocytic outcomes of drug treatment (i). The mechanism which underpins HDAC6 and its effect on HR23B involves the interplay with the HSP90 (via HDAC6 BUZ domain interacting with HR23B UbL domain; ii). HDAC6 has an established role in activating HSP90 chaperone function through its catalytic (CAT) domain.

FIG. 6

Either A2058 (a and c) or A375 (b and d) cells were treated with the indicated inhibitors SAHA or VPA and after 24 hr immunoblotted for HR23B, LC3 or actin antibody as indicated.

e) U2OS cells were treated with HR23B or control (NT) siRNA (20 nM for 72 hr) in the presence (+) or absence (−) of SAHA (10 μM for 18 hr as indicated), and subsequently immunoblotted with anti-HR23B, anti-LC3 and anti-actin antibody.

f) RT-PCR of HR23B RNA in A549 control (WT) and HDAC6 (KD) cells. GAPDH was used as control.

g) Flag-HDAC6 stable cells were either uninduced (−) or induced (+) with doxycycline (for 72 h) and then treated with bortezomib (to block proteasome activity; indicated in nM) for 48 hr. Cell extracts were immunoblotted with anti Flag, HR23B or actin antibodies as indicated.

FIG. 7: Expression of HR23B and LC3 in patients treated with HDAC inhibitors, and prognostic significance of HR23B and LC3

Representative images of HR23B and LC3 immunohistochemistry in colorectal cancer biopsies showing examples of tumours expressing high HR23B/low LC3 (7a) and low HR23B/high LC3 (7b). Magnification as indicated.

c) Kaplan-Meier disease free survival (DFS) plots in colorectal adenocarcinomas based on low or high expression of HR23B (assessed using TSS). The 5 year disease free survival for each group is also shown. Log-rank test p<0.001.

d) Kaplan-Meier DFS plots in colorectal adenocarcinomas based on low or high expression of LC3 (assessed on percentage of tumour cells expressing LC3). The 5 year disease free survival for each group is also shown. Log-rank test p<0.001.

FIG. 8: Structure of HR23B

A schematic diagram of the domain structure of HR23B. Running from the N-terminus to the C-terminus, HR23B comprises the following domains: ubiquitin, SPTA rich, UBA, XPC binding and UBA. The word “Immunogen” indicates that the shaded area above (the SPTA rich domain) is an example of a domain against which an anti-HR23B antibody may be directed.

FIG. 9: Prognostic significance of HR23B and LC3 expression in combination on overall survival

a) Kaplan-Meier overall survival plots in colorectal cancer patients based on low or high expression of HR23B in combination with low or high expression of LC3 (assessed using TSS). Analysis time is shown in months.

b) Table showing 3-year overall survival showing 95% confidence intervals.

c) Table showing 5-year overall survival showing 95% confidence intervals.

d) Results of three separate univariate Cox models estimating effect of biomarker on overall survival. Hazard ratios are in comparison to a negative (0) score. The multivariate analysis presents the results after adjustment for the relevant clinical prognostic variables (*age at randomisation, gender, chemotherapy, radiotherapy, treatment, disease site and stage were also included in the multivariate analysis).

FIG. 10: Prognostic significance of HR23B and LC3 expression in combination on disease free survival (DFS)

a) Kaplan-Meier disease free survival in colorectal cancer patients based on low or high expression of HR23B, in combination with low or high expression of LC3 (assessed using TSS). Analysis time is shown in months.

b) Table showing 3-year disease free survival showing 95% confidence intervals.

c) Table showing 5-year disease free survival showing 95% confidence intervals.

d) Results of three separate univariate Cox models estimating effect of biomarker on disease free survival. Hazard ratios are in comparison to a negative (0) score. The multivariate analysis shows the results after adjustment for the relevant clinical prognostic variables (*age at randomisation, gender, chemotherapy, radiotherapy, treatment, disease site and stage were also included in the multivariate analysis).

### EXAMPLES

### Example 1

### Materials and Methods

### Cell Culture

Cells were cultured at 37° C. in a humidified 5% CO₂ incubator in DMEM (Invitrogen) containing 10% FCS (U2OS, A2058, A375, HCT116, HCT15, WT and shRNA HDAC6 A549 cells), or RPMI containing 20% FCS (HUT78, MYLA). All media contained 1% penicillin/streptomycin (Invitrogen). U2OS-TET-Flag-HR23B and HDAC6 inducible cell lines were grown in DMEM containing 10% tetracycline-negative FCS (PAA Laboratories), hygromycin (Invitrogen), G418 (Promega) and 1% penicillin/streptomycin (Invitrogen). Flag-HR23B and Flag-HDAC6 was induced by the addition of 1 μg/ml doxycycline to culture media.

A U2OS-TET-Flag-HDAC6 inducible cell line was created by selecting cells transfected with Flag-HDAC6 inserted in a TET-ON gene expression system (Clontech). The shRNA A549 HDAC6 WT and KD cells were a kind gift from T. P. Yao (49).

### Plasmids

pcDNA-Flag-HDAC6, ΔBUZ, ΔN (439-1215) and 1-503 were a kind gift from T. P. Yao (49). BUZ was amplified from wild-type pcDNA-Flag-HDAC6 with the following primer combination: Forward (5′-CTACTAGCGGCCGCAATGACCTCAACACGC-3′) and Reverse (5′-TAGTAGGCGGCCGCTTATTTATCATCATC-3′) and then inserted into the pcDNA3.1 vector. pcDNA3.1a-ubiquitin (His6-tagged) was a gift from Dr. Dimitris Xirodimas (CRBM, Montpellier, France). HR23B wild-type and the truncated derivatives ΔUbA and ΔUbL were amplified from wild-type HR23B with the following primer combinations: HR23B wild-type with UbL Forward (5′-GTGGTGGAATTCATGCAGGTCACCCTGAAGACCC-3′) and UbA Reverse (5′-20TCTAGACTCGAGATCTTCATCAAAGTTCTGCTGTAG-3′); ΔUbA with UbL Forward (5′-GTGGTGGAATTCATGCAGGTCACCCTGAAGACCC-3′) and UbL Reverse (5′-TCTAGACTCGAGAGACTGACCCGTCACAAGTGC-3′); ΔUbL with UbA Forward (5′-GTGGTGGAATTCATGGCAGTGTCCACACCAGCACCAG-3′) and UbA Reverse (5′-TCTAGACTCGAGATCTTCATCAAAGTTCTGCTGTAG-3′).

The PCR products of HR23B wild-type and ΔUbA and ΔUbL mutants were inserted into the pcDNA3.1 (+)/Myc-His vector (Invitrogen) via the restriction sites EcoRI and XhoI using the Liga Fast rapid DNA ligation system (Promega) in order to create myc/his tagged constructs.

Transfection, siRNA and Compound Treatment

Cells were transfected with siRNAs against HR23B (5′-GAUGCAACGAGUGCACUUG-3′), HDAC6 (00349900; Dharmacon) or non-targeting control #2 (Dharmacon, Perbio), as indicated using oligofectamine (Invitrogen) to a final concentration of 50 nM before harvesting. Plasmids were transfected using Genejuice (Merck) according to manufacturer instructions. Cells were treated with superoylanilide hydroxamic acid (SAHA), valproic acid, 17-AAG, 3-MA or MG132 (Chemietek) at the indicated concentrations or conditions and for the indicated times before harvesting.

### Purification of Proteins Modified by Ubiquitin

Transfected cells in a 10 cm dish were washed twice with ice-cold PBS and harvested into 1 ml of 8M urea, 0.1 M Na₂HPO₄/NaH₂PO₄, 0.01M Tris-HCl, and pH8.0. Lysates were sheared by taking through 23 gauge needle to reduce viscosity. Protein concentration was measured with Bradford (Bio-Rad) and normalised. About 100 μl of lysate was mixed with 4×SDS loading buffer for input. Then 900 μl of lysate was incubated with 70 μl of a 50% slurry of nickel (Ni²⁺-NTA)-agarose beads (Qiagen), 5 mM imidazole and 10 mM β-mercaptoethanol overnight at 4° C. The beads were collected by 1 min centrifugation at 8,000 rpm and washed with 750 μl of the following buffers with 5 min mixing at room temperature for each washing step: twice with 8M urea, 0.1M Na₂HPO₄/NaH₂PO₄, 0.01 M Tris-HCl, pH 8.0, 10 mM β-mercaptoethanol and 0.1% Triton X-100; three times with 8M urea, 0.1M Na₂HPO₄/NaH₂PO₄, 0.01M Tris-HCl, pH 6.3, 10 mM β-mercaptoethanol and 0.1% Triton X-100. The bound proteins were then eluted with 100 μl of elution buffer (200 mM imidazole, 5% SDS, 150 mM Tris-HCl pH6.8, 30% glycerol, 720 mM β-mercaptoethanol, 0.005% bromophenol blue) for 30 min incubation followed by 10 min spin at 13,000 rpm. Samples were heated to 95° C. for 5 min, cooled, and a typical volume of 30 μl loaded on each lane for immunoblot analysis.

### Flow Cytometry

Cells were harvested using trypsin and washed in phosphate buffered saline (PBS) and fixed in 70% ethanol/PBS. To analyse, samples were incubated for 30 min with RNAse A and 20 ng/ml propidium iodide (Sigma) at 4° C. Samples were run on a FACScan flow cytometer (BD Bioscience) and the analysis was carried out using CellQuestPro software. The population of sub-G1 DNA content cells is presented.

### Immunoblotting and Immunoprecipitation

Cells were harvested, washed in PBS and lysed in RIPA lysis buffer (50 mM Tris pH 7.4, 150 mM NaCl, 1 mM EDTA, 0.25% sodium deoxycholate, 1.0% NP-40, and protease inhibitors) at 4° C. for 20 min. Cell lysate was normalised (Bradford assay) and equal protein loading was confirmed with Ponceau S staining. For immunoprecipitation, cells were lysed in TNN buffer (50 nM Tris pH 8, 120 nM NaCl, 0.5% NP-40, 1 mM dithiothreitol, and protease inhibitors) and incubated overnight at 4° C. with the appropriate antibody bound to protein G-Sepharose beads. Total protein was resolved by denaturing SDS-PAGE gel electrophoresis before transfer to nitrocellulose membrane (Inverclyde Biologicals) and probed with antibody overnight at 4° C. Membranes were incubated with corresponding secondary HRP-antibodies for 1 h at room temperature before signal was detected using enhanced chemi-luminescence (Perbio). Antibodies used were anti-HR23B and PARP (uncleaved and cleaved; CellSignalling), LC3 (Nanotools), Cathepsin D (Sigma), LAMP1 (Santa Cruz), HDAC6 (Millipore), Myc (9E10) (Santa Cruz Biotechnology), Flag-M2 and actin (Sigma), Ku80 (Cell Signalling), HSP90α/β (Santa Cruz), GRP78 (Santa Cruz), tubulin (Santa38Cruz), acetylated tubulin (Sigma) and cleaved Caspase 3 (Cell Signalling). Anti-ubiquitin FK2 mouse antibody was purchased from BIOMOL.

### Immunofluorescence

Cells seeded onto coverslips were washed in PBS and fixed in 3.7% formaldehyde for 15 min. Cells were permeabilised in PBS containing 0.5% Triton X-100 for 5 min and then blocked in 5% FCS/PBS for 30 min at room temperature before incubation with primary antibody as indicted. Coverslips were washed in PBS containing 0.05% Tween-20 and incubated with corresponding secondary antibodies for 30 min at room temperature. Alexa Fluro 488 (green fluorescence) or Alexa Fluor 594 (red fluorescence) (Molecular Probes). Finally coverslips were washed extensively in PBS containing 0.05% Tween-20 and mounted onto microscope slides using Vectashield containing DAPI (Vector Laboratories).

### Cell Colony Assay

U2OS-TET-Flag-HR23B inducible cells were seeded at 1000 cells/well in 6-well plates. Flag-HR23B was induced for 4 hr prior to 24 hr drug treatment as indicated. At 9 days post-drug treatment, cells were washed in PBS, fixed in methanol and stained with crystal violet solution for 15 min before extensive washing with water. The colonies were quantified using an Alphalmager® HP Imaging System and Alphaview software by Alpha Innotech Corporation.

### Expression and Immunoprecipitation for Mass Spectrometry

The U2OS TET-Flag-HDAC6 inducible or TRE vector control cell lines were induced with doxycycline 1 μg/ml for 60 hr before harvesting. Cells were harvested, washed twice with ice-cold PBS and lysed in 1.5 ml lysis buffer (0.5% NP40, 100 mM NaCl, 20 mM Tris, 10 mM MgCl₂, pH 7.4) for 1 hr. After removing the debris by centrifugation at 13000 rpm for 10 min, the supernatant was collected and 10% of the total protein was separated for immunoblot analysis. The protein lysate was loaded onto Flag-tagged beads (Sigma), pretreated with dimethyl adipimidate in order to prevent non-specific elution of Ig heavy chain. The beads were incubated with the cell lysate for 3 hr at 4° C. After washing 4 times with ice-cold lysis buffer and once with ice-cold PBS, the bound proteins were eluted by boiling the beads for 5 minutes in 2× Laemmli buffer. After transferring the supernatant to a new tube, β-mercaptoethanol was added to a final concentration of 2%.

Half of the eluates were separated on NuPAGE gradient gels (Invitrogen), fixed and stained with Instant Blue according to the manufacturer's instructions (Expedeon). Enriched stained bands present in the HDAC6 sample as compared to the vector only control were excised and put into a prelubricated microcentrifuge tube (FisherScientific). Preparation of gel slices, reduction, alkylation and in-gel protein digestion was carried out as described (51, 52) except that the alkylation was performed with chloroacetamide instead of iodoacetamide. The peptides were desalted, filtered, and enriched on C18 tips (Sigma Aldrich).

### Analysis of Mass Spectrometry Results

Peptides and proteins were identified by Mascot (v2.3.01 CBRG Cluster) via automated database searching of all MS/MS spectra against the UniProt SwissProt swissprot/uniprot_sprot.v2012.07.13 homo sapien database (20,319 sequences). The data were analysed with the following search parameters: Type of search: MS/MS IonSearch; Enzyme: Trypsin; Fixed modifications: Carbamidomethyl (C); Variable modifications: Oxidation (M), Deamidated (NQ); Mass values: Monoisotopic; Protein mass: Unrestricted; Peptide mass tolerance: ±1.8 Da (#13C=1); Fragment mass tolerance: ±0.6 Da; Max missed cleavages: 3; Instrument type: ESI-TRAP. After removing known contaminants from the list of detected proteins, only those proteins which have individual ion scores of at least 40 and for which at least two significant peptide matches were identified, were accepted as potential HDAC6 interacting proteins. The UniProt accession numbers were searched against the STRING database version 9.0 (Jensen et al., 2009) for protein-protein interactions. Only interactions between the proteins belonging to the dataset were selected. STRING defines a metric called confidence score to define interaction confidence; all interactions for the dataset were chosen which had a confidence score ≧0.4. The clustering was performed via the MCL option which accepts a parameter called “inflation” and was set to 2. Further analysis and the determination of GO enrichments were carried out with Cytoscape 2.8.3 (Shannon et al., 2003) and plugin BiNGO 2.44 (Maere et al., 2005) and Gephi 0.8.1 (53). The following parameters were applied for the BiNGO search: Hypergeometric test; Benjamini and Hochberg false discovery rate correction; significance level: 0.05; GO biological process; organism: Homo sapiens.

### Example 2

### HR23B Influences Autophagy

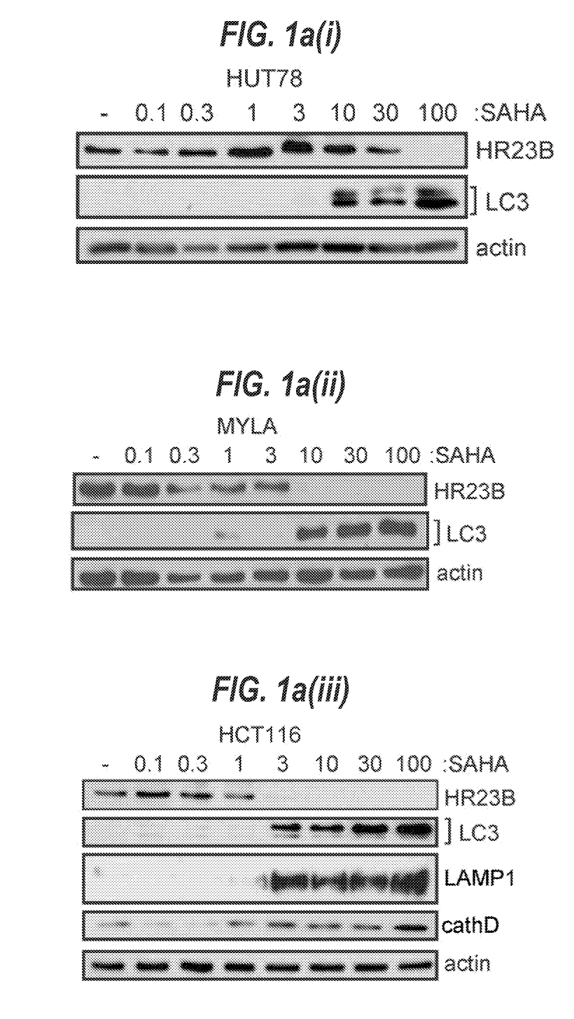

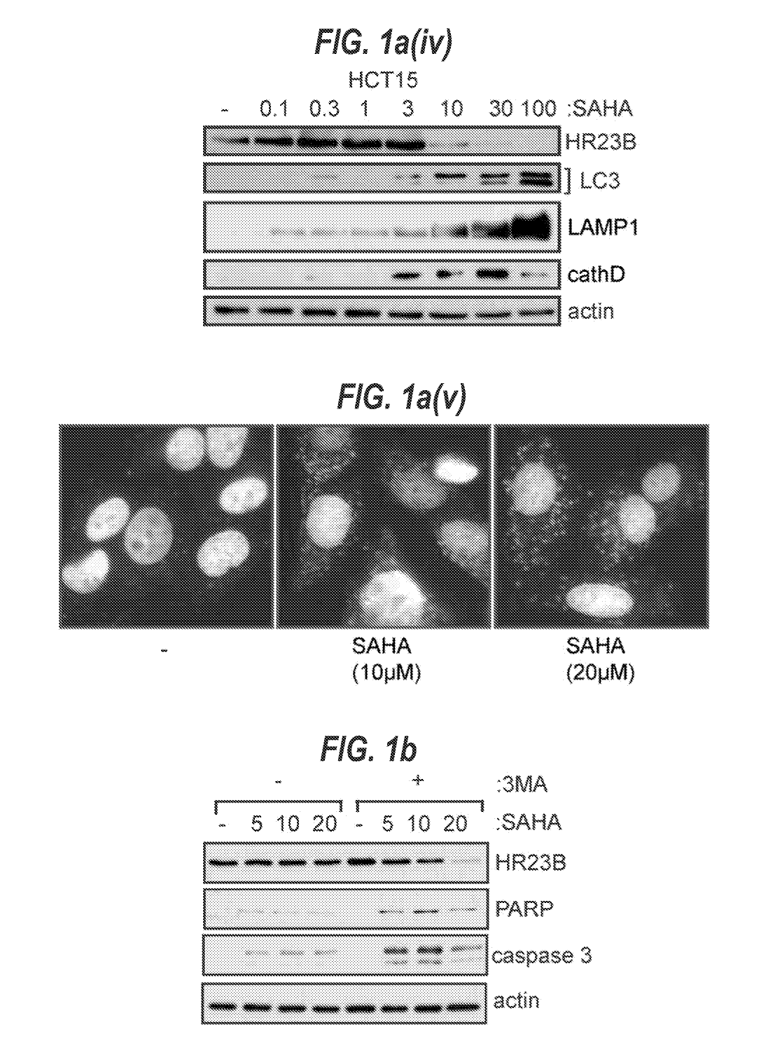

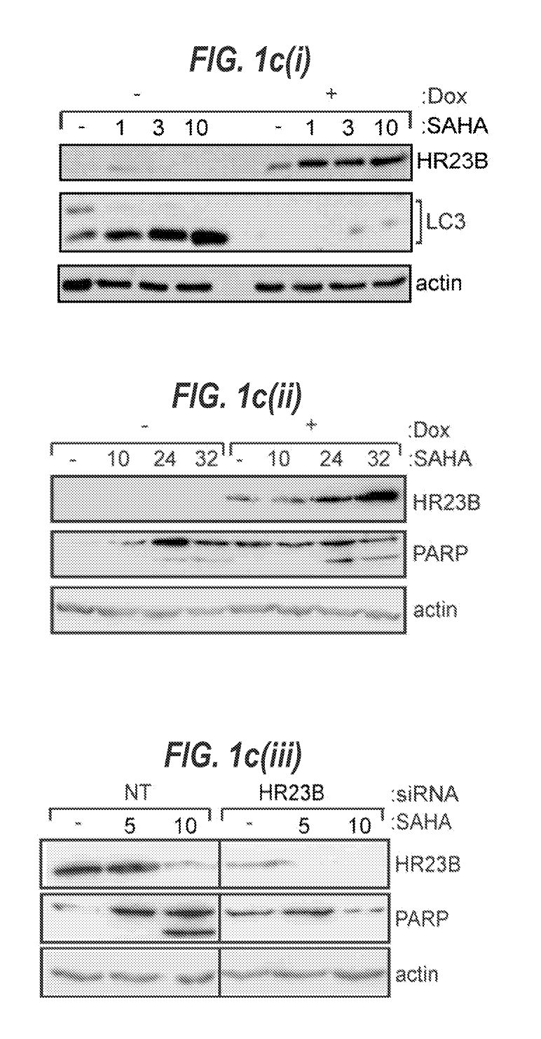

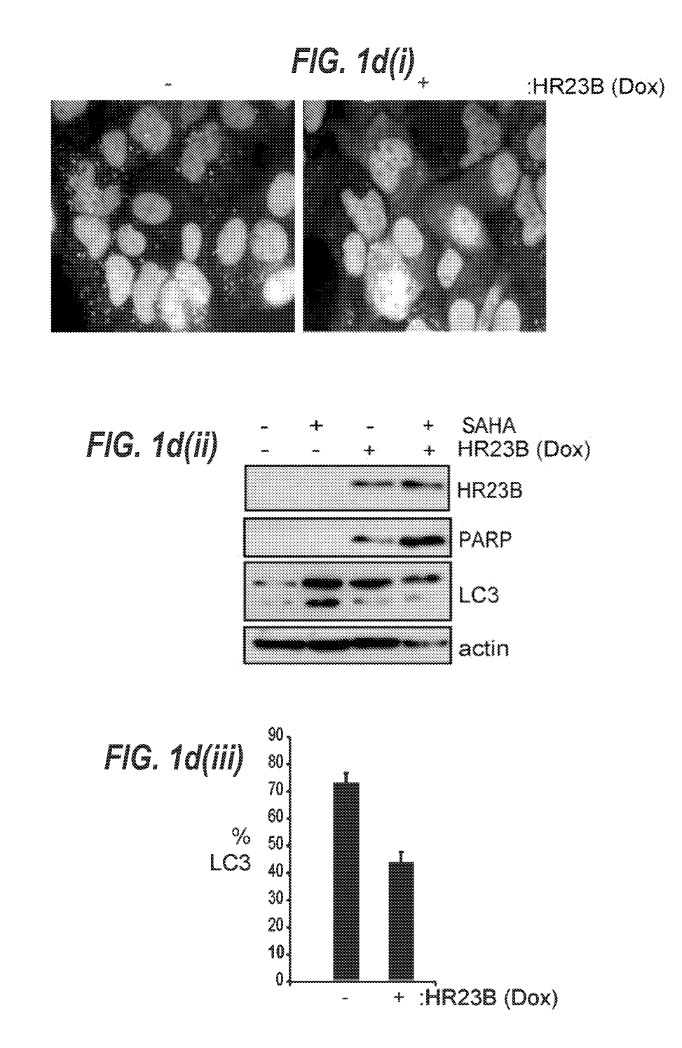

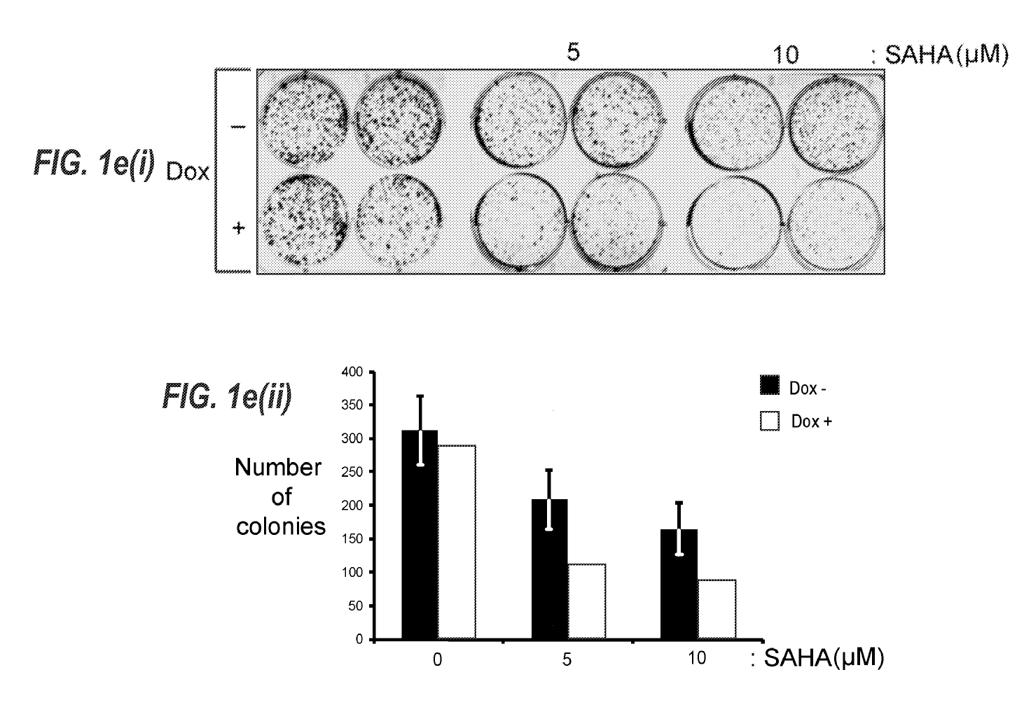

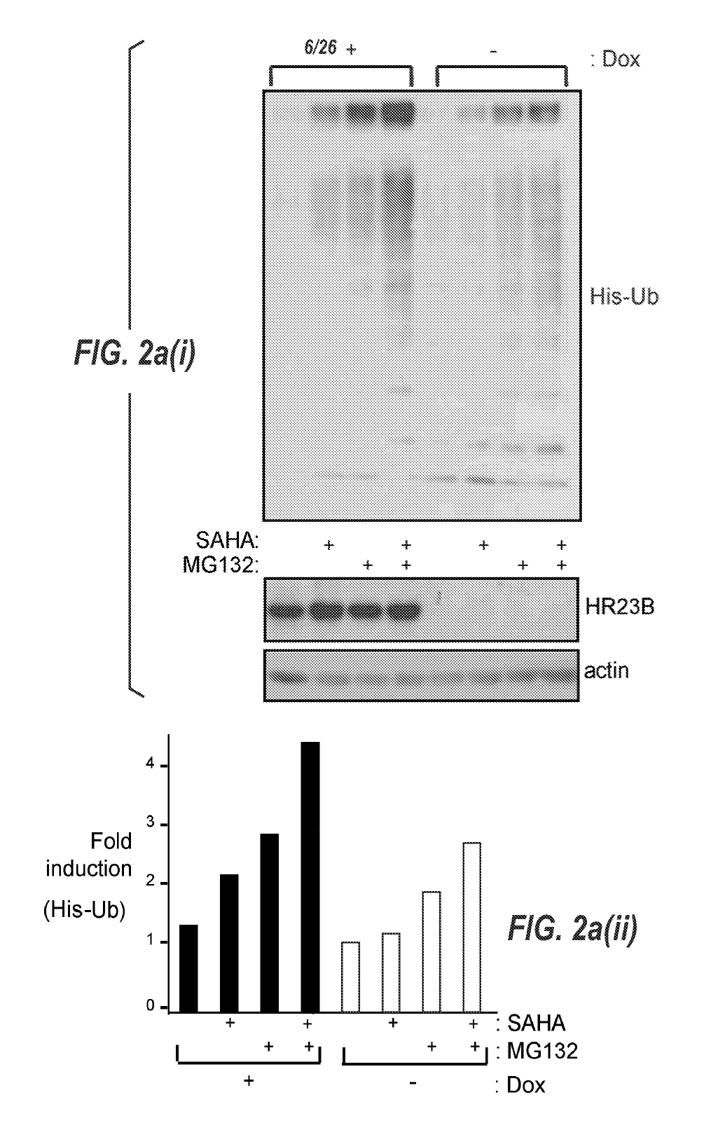

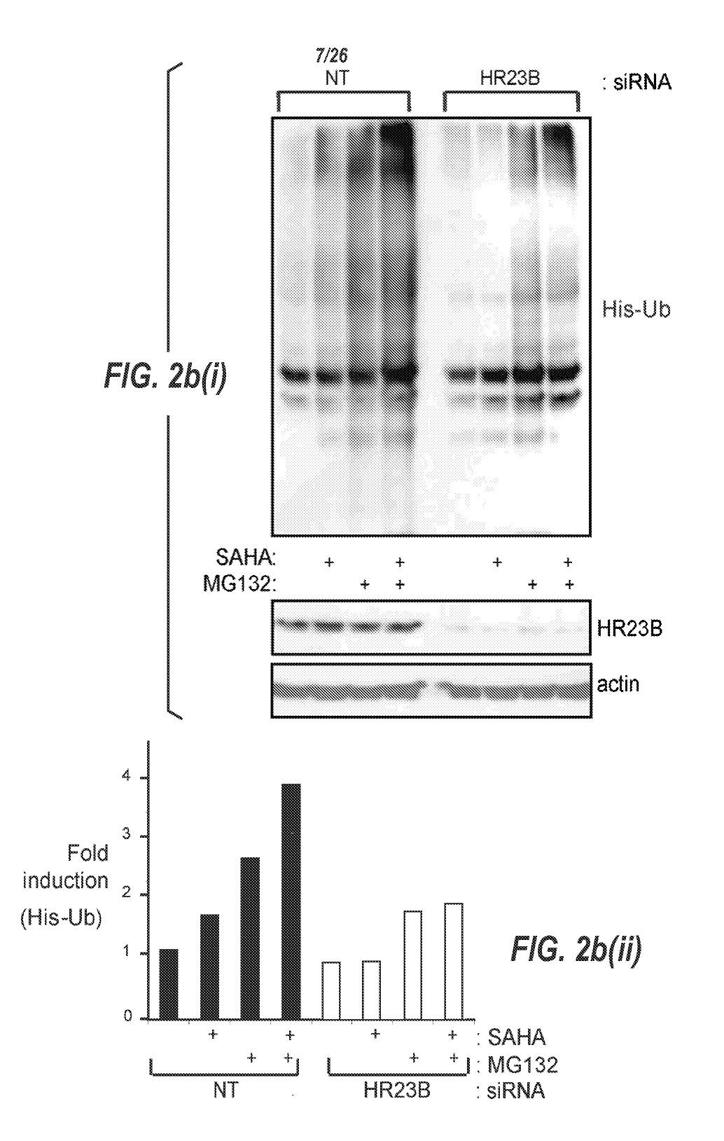

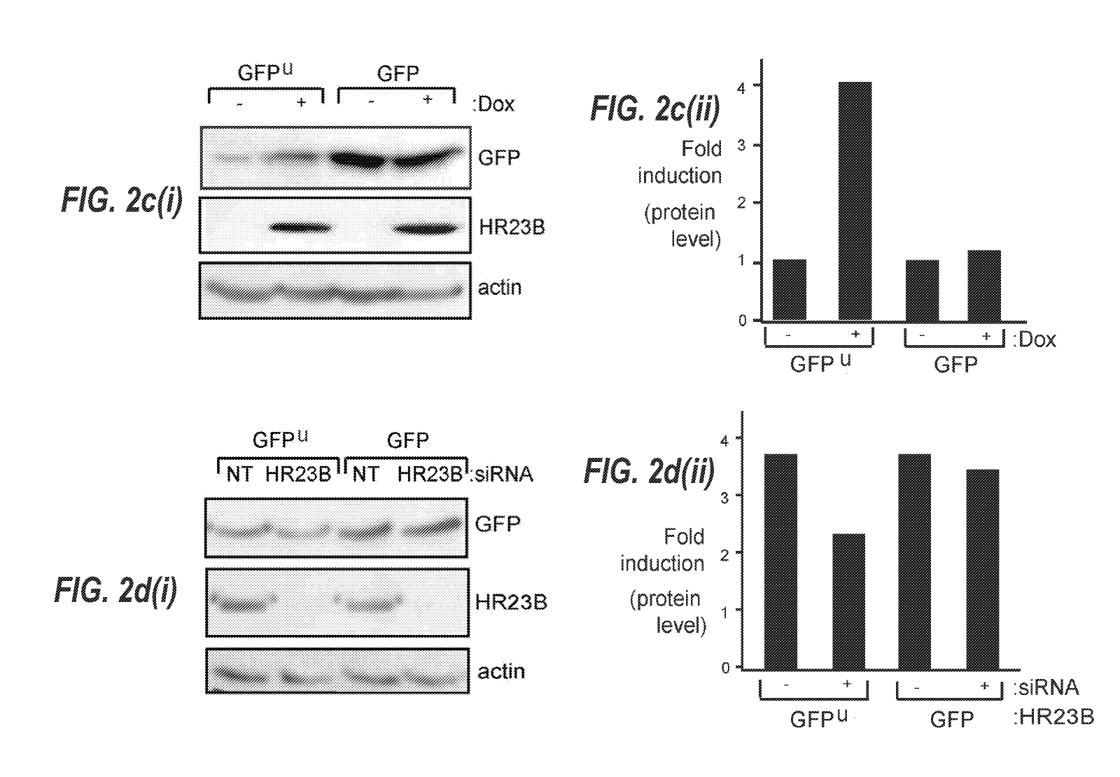

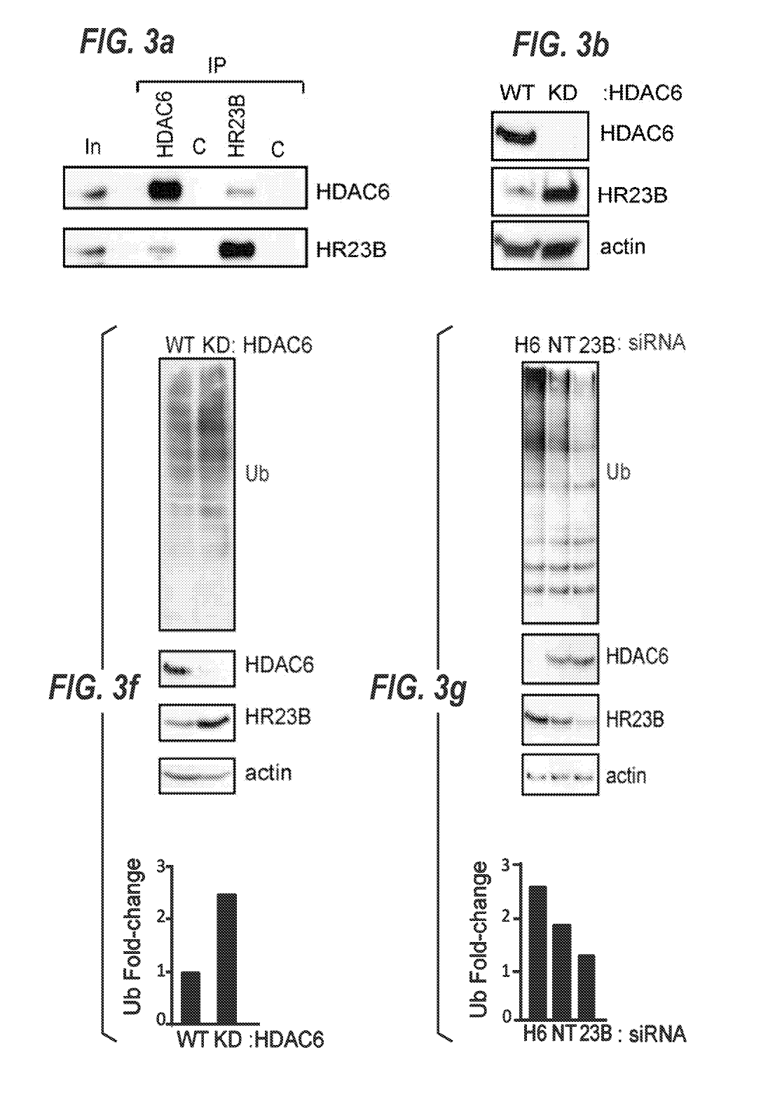

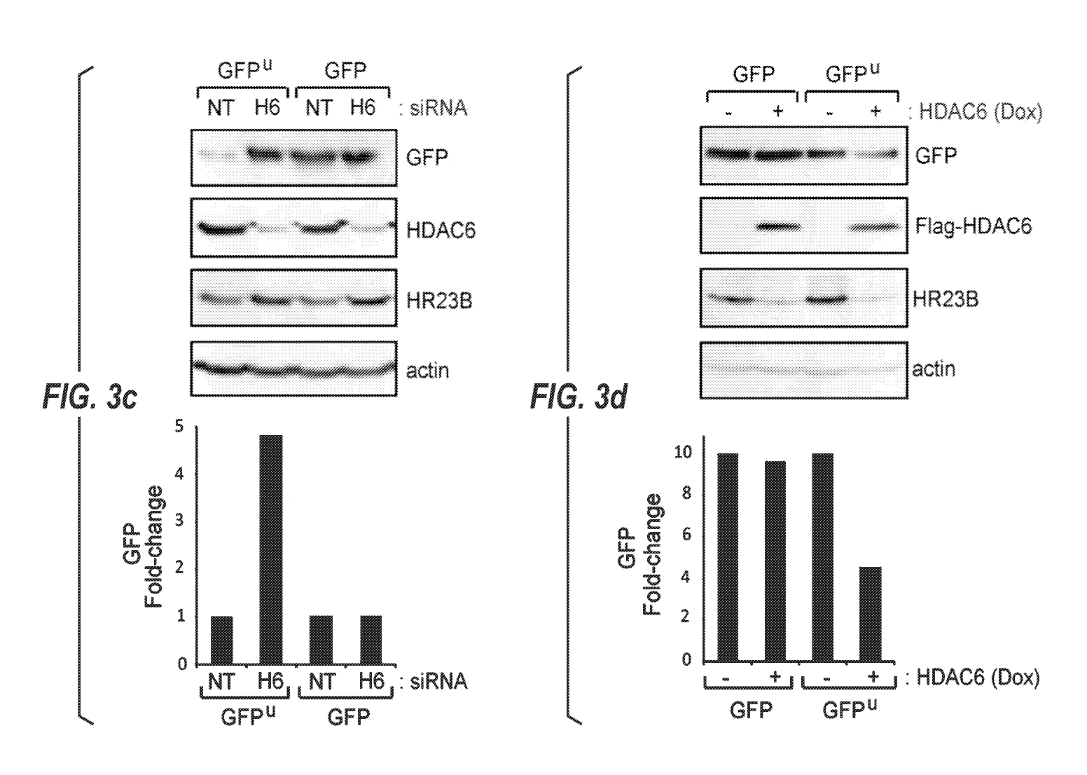

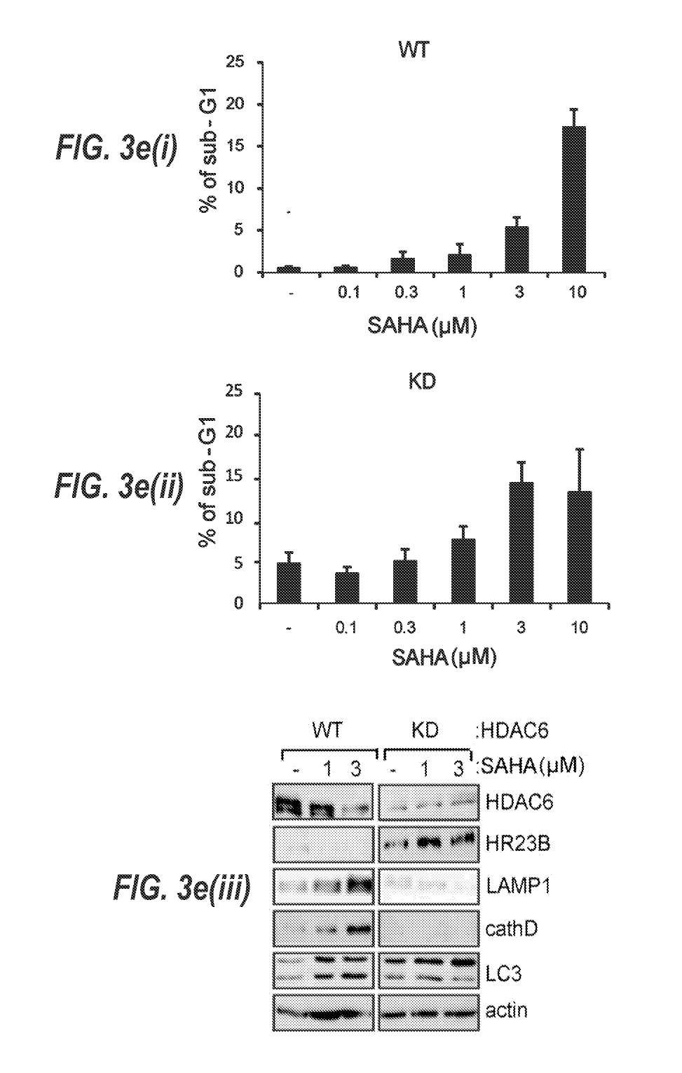

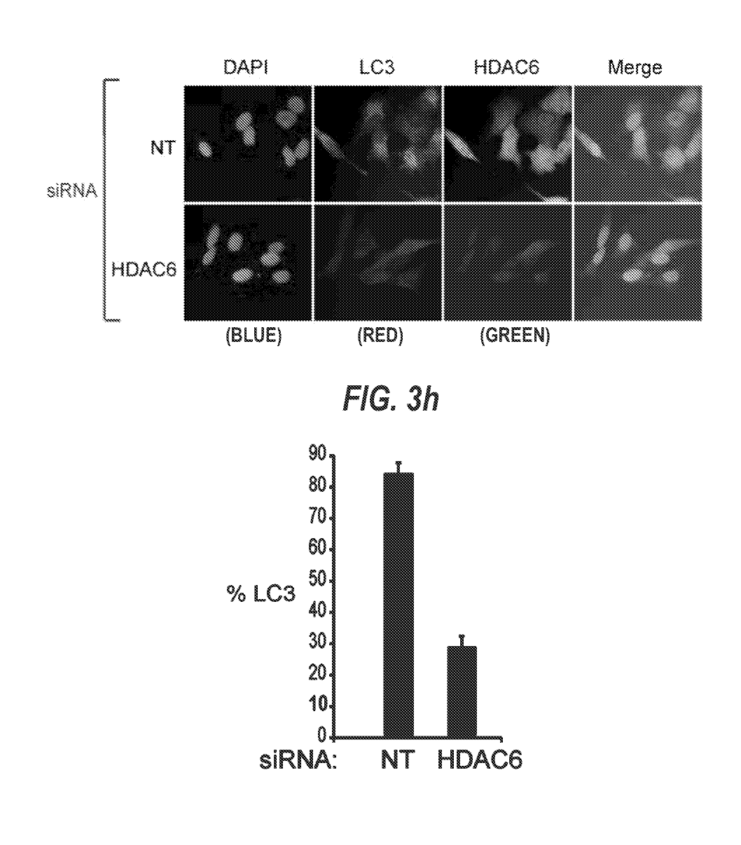

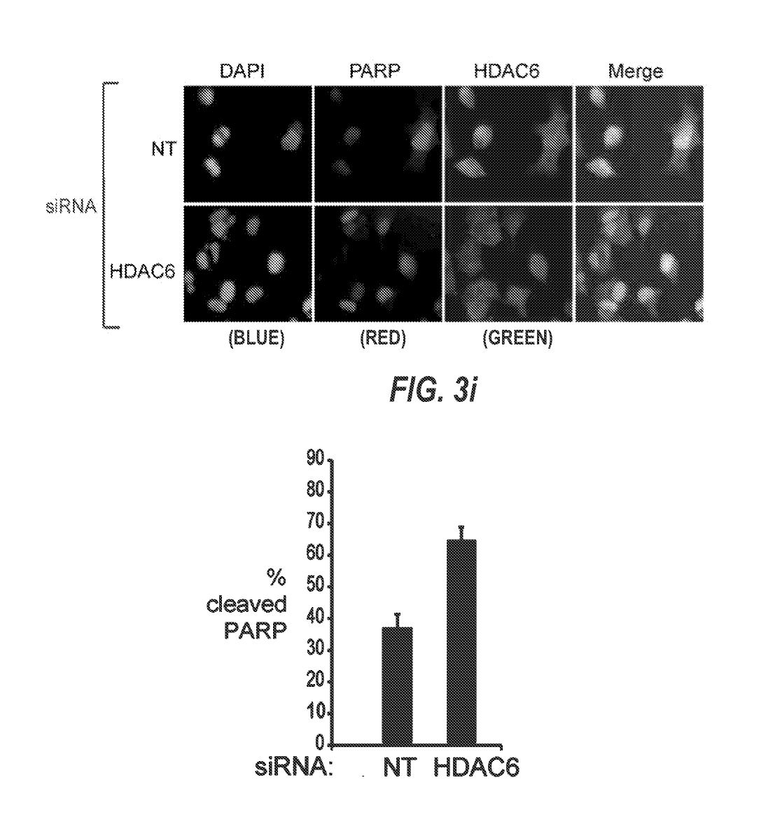

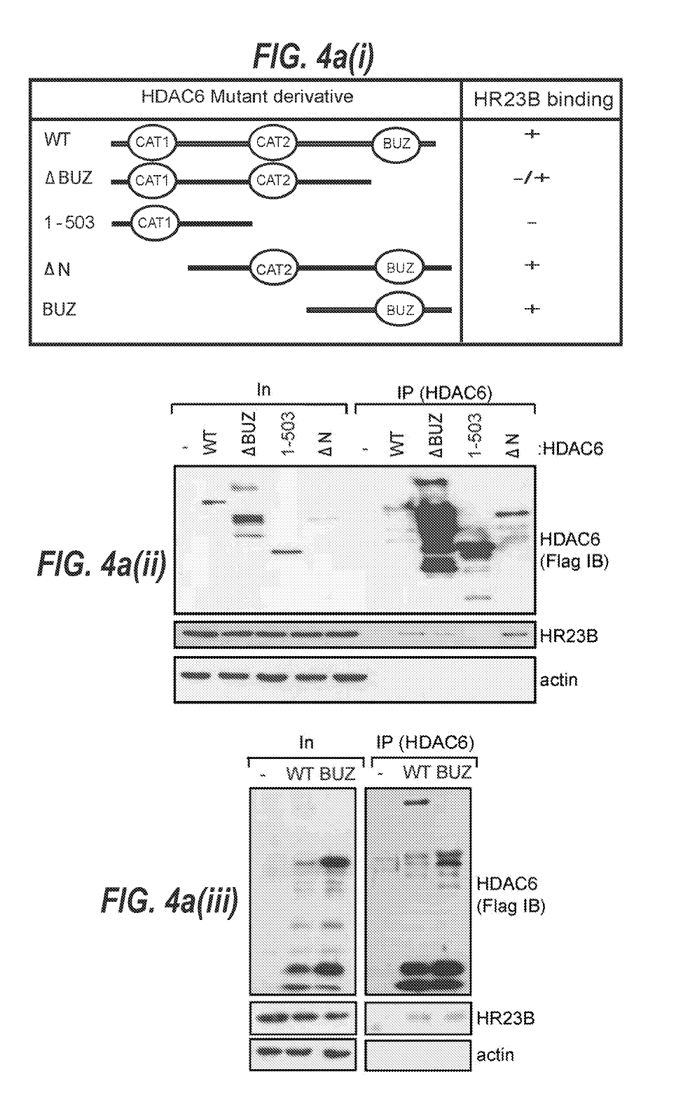

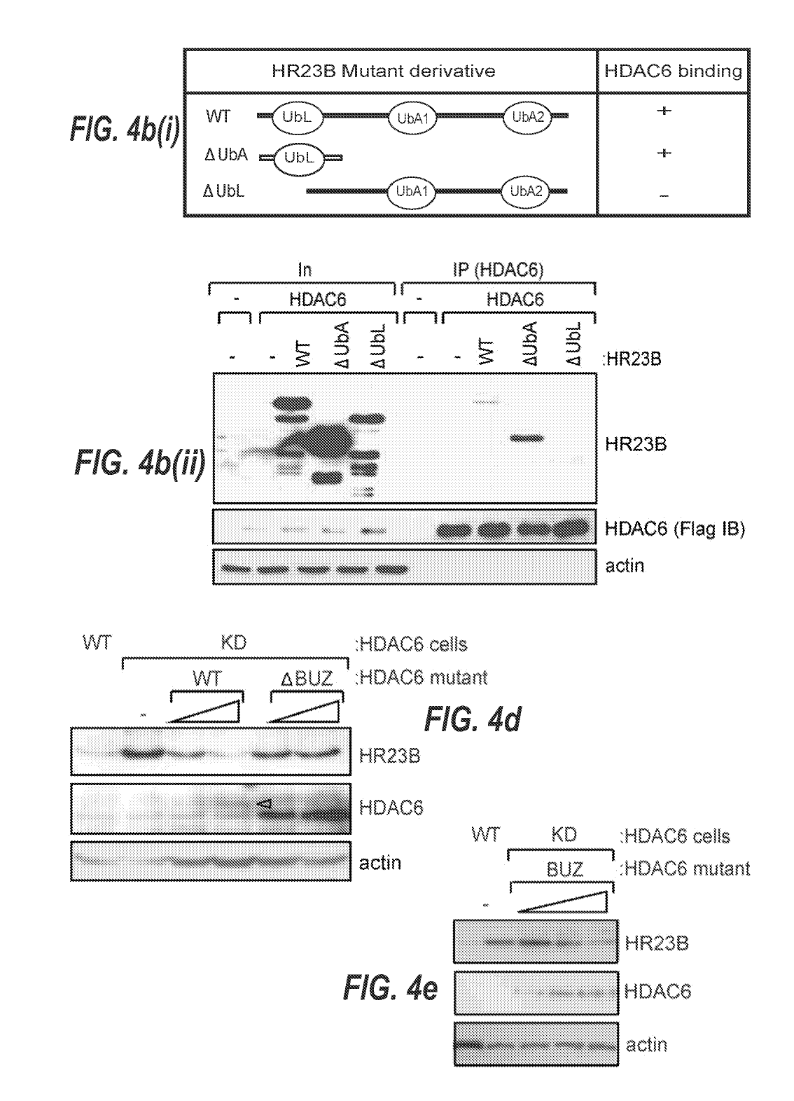

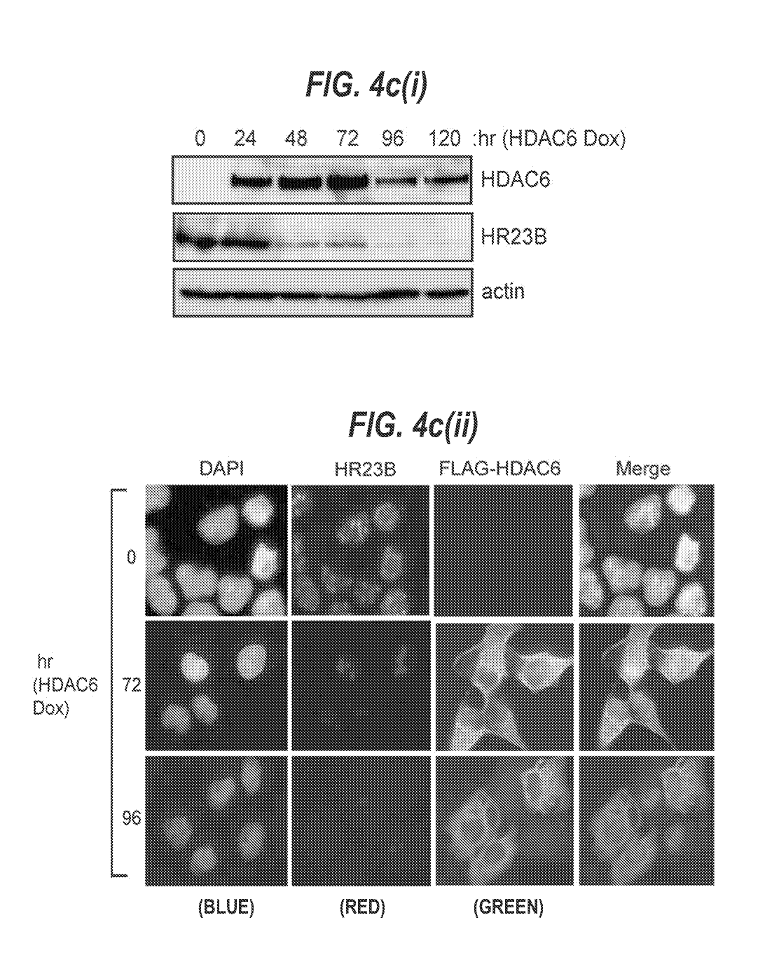

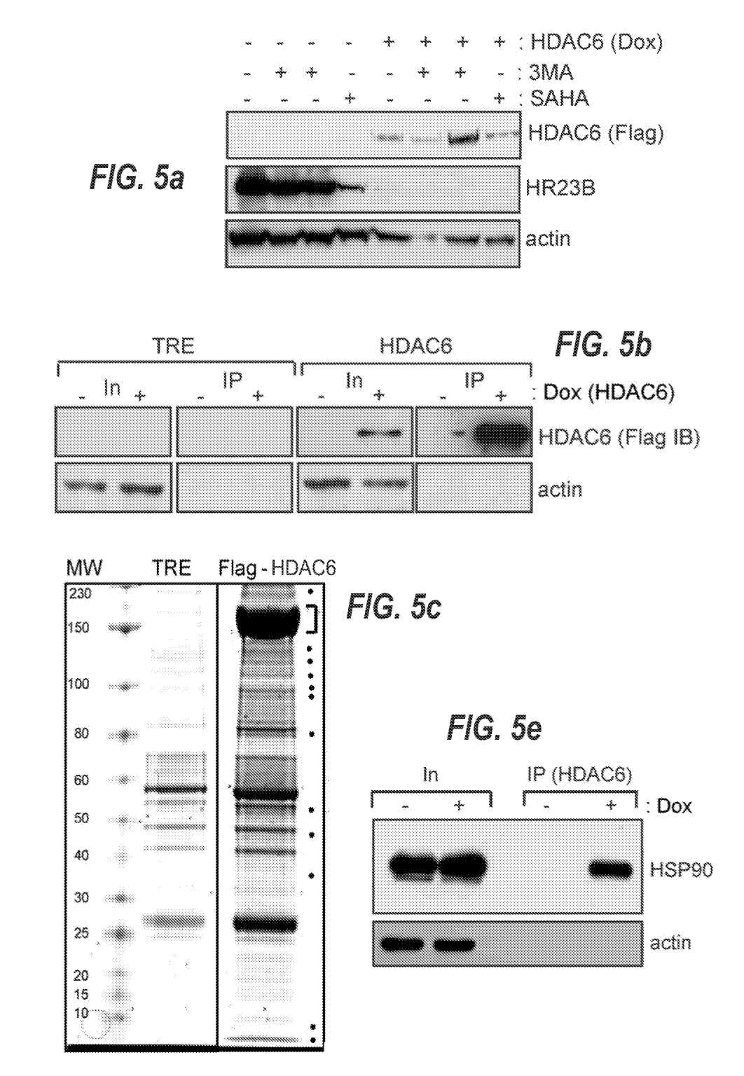

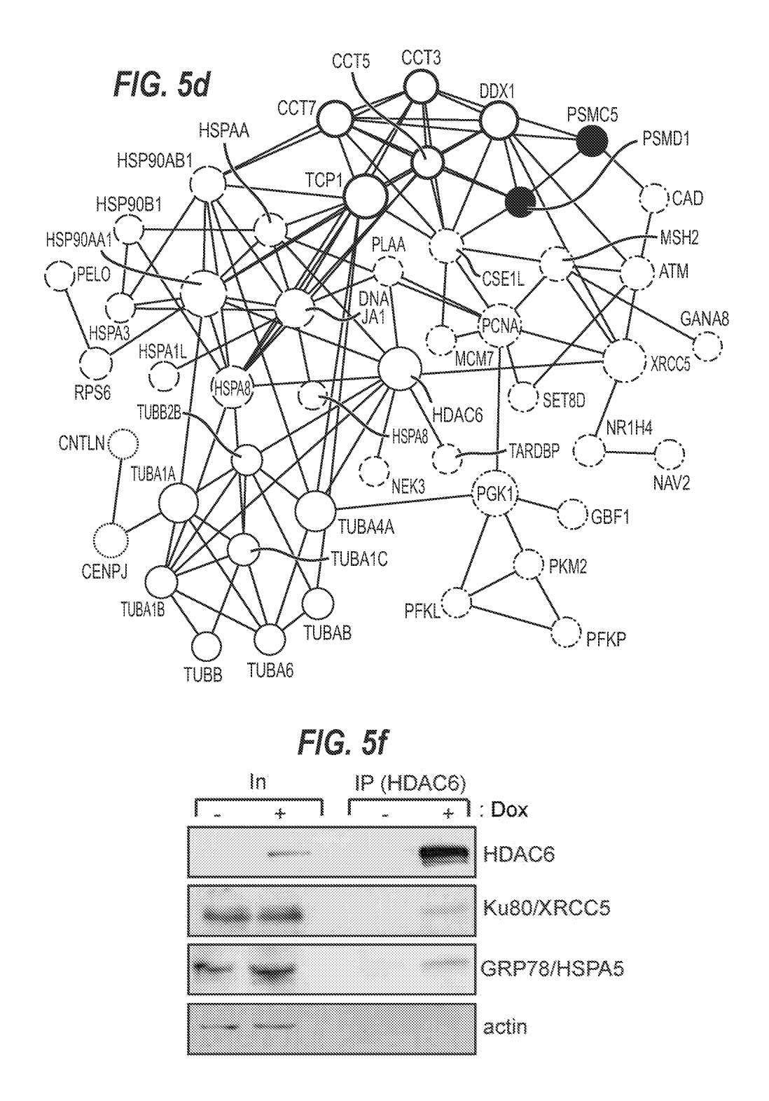

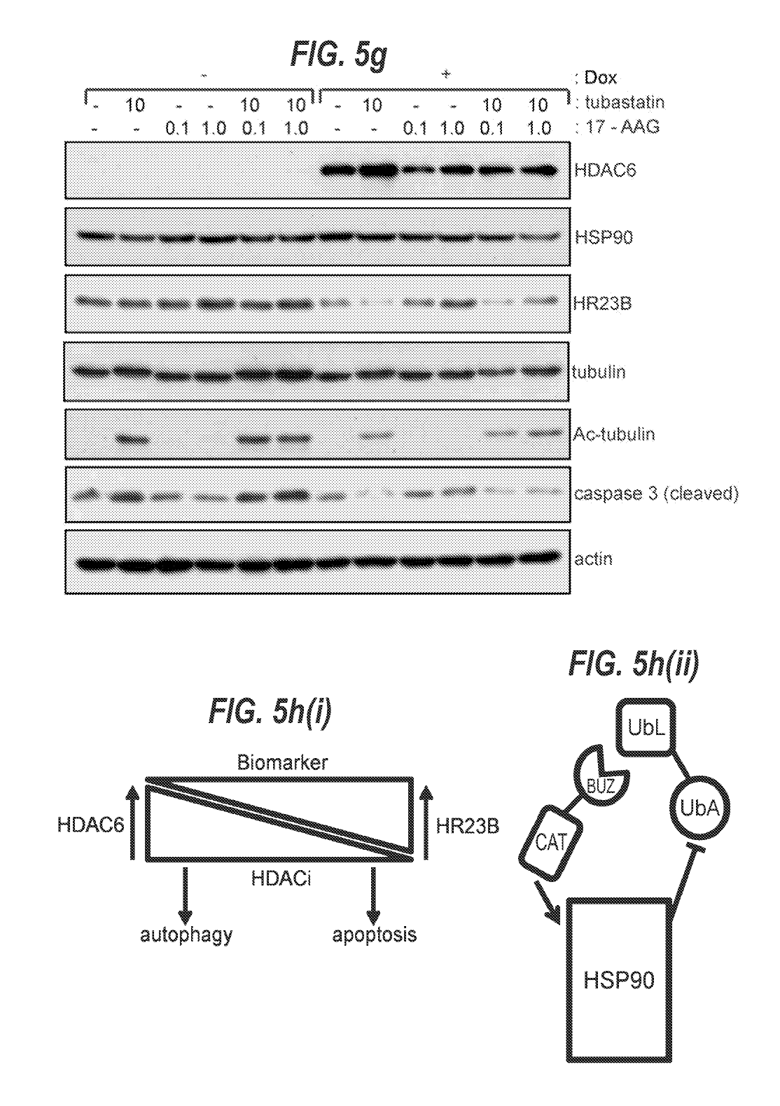

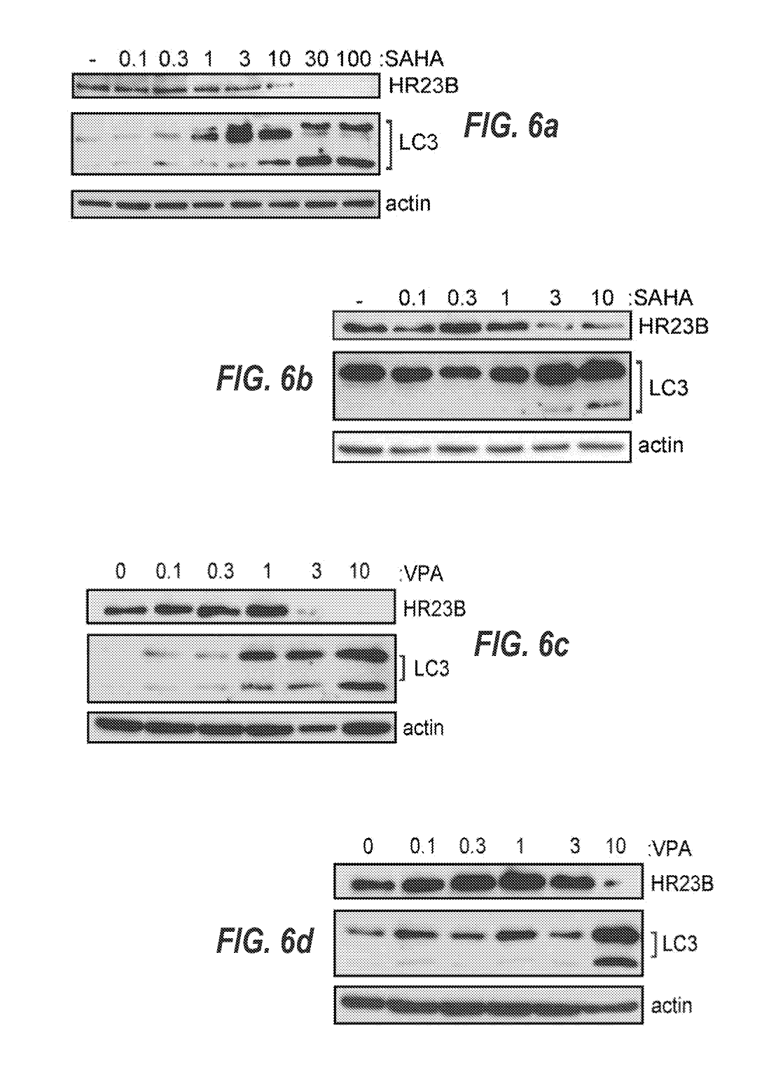

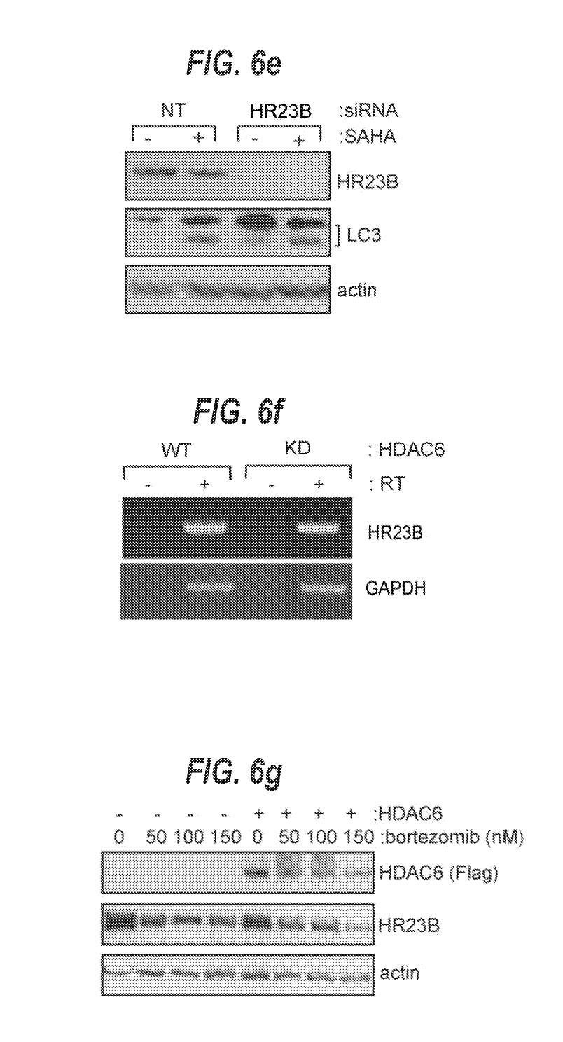

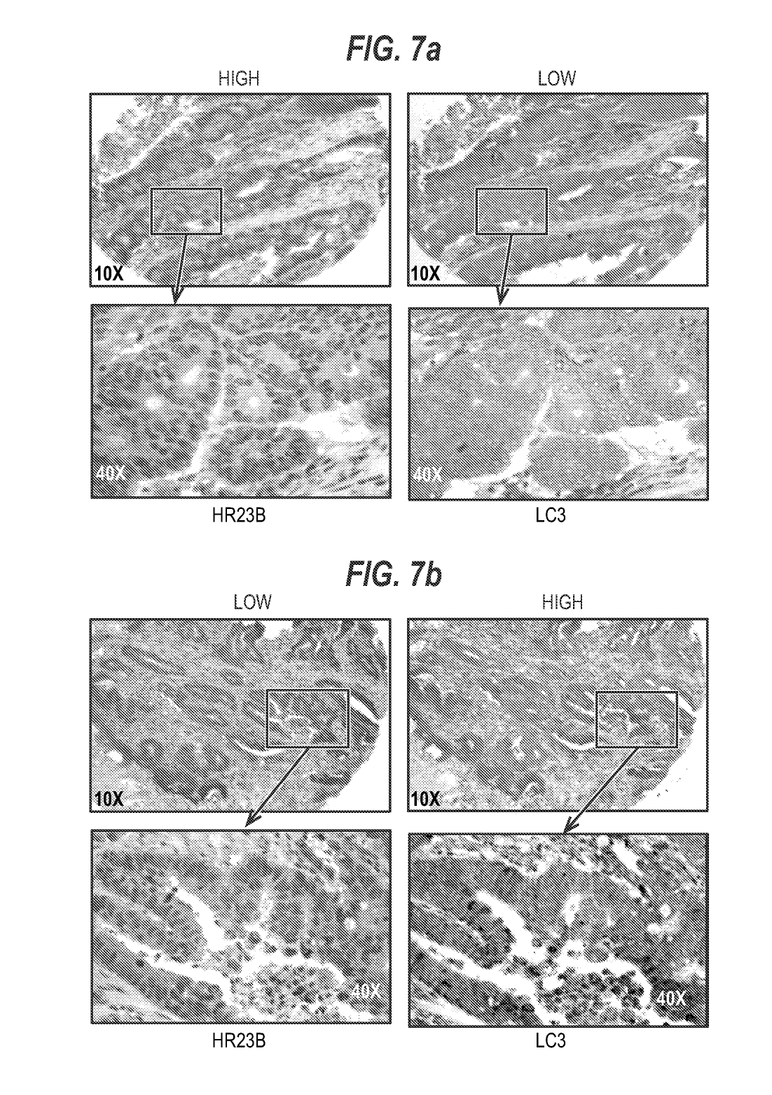

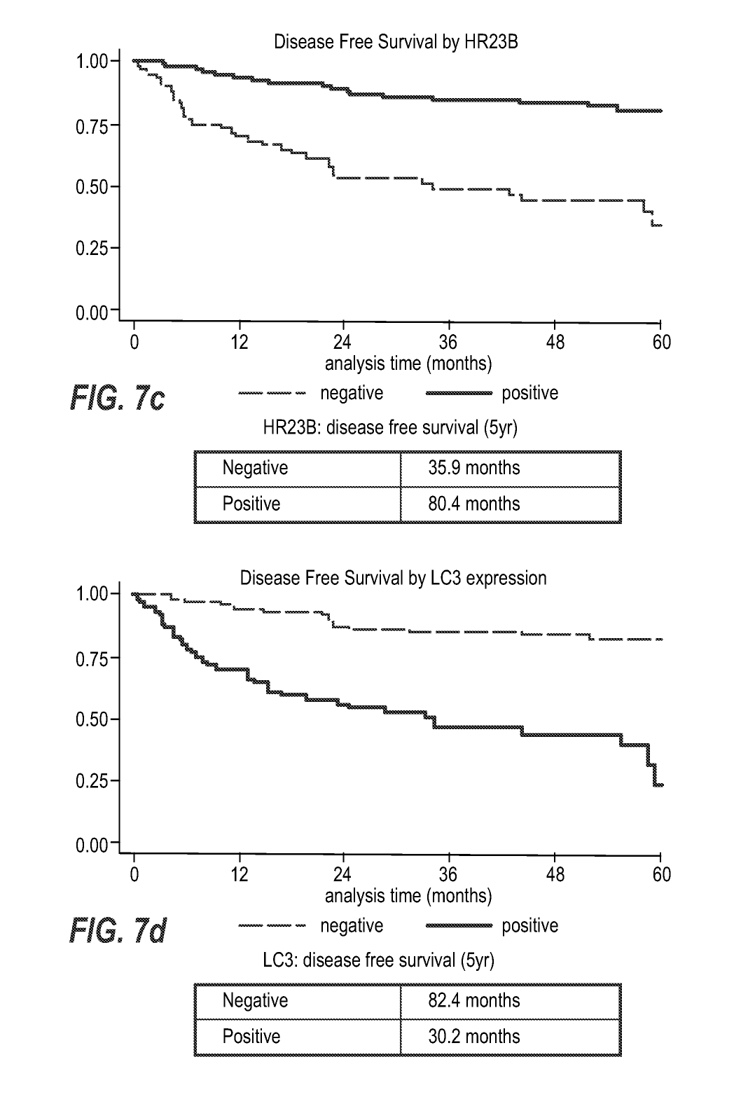

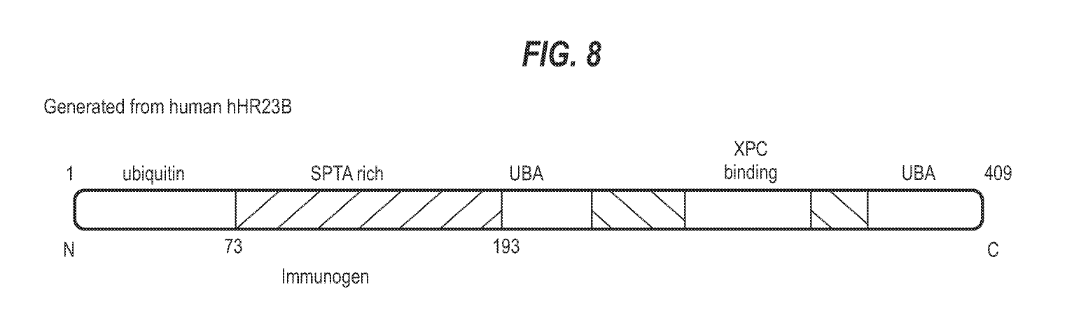

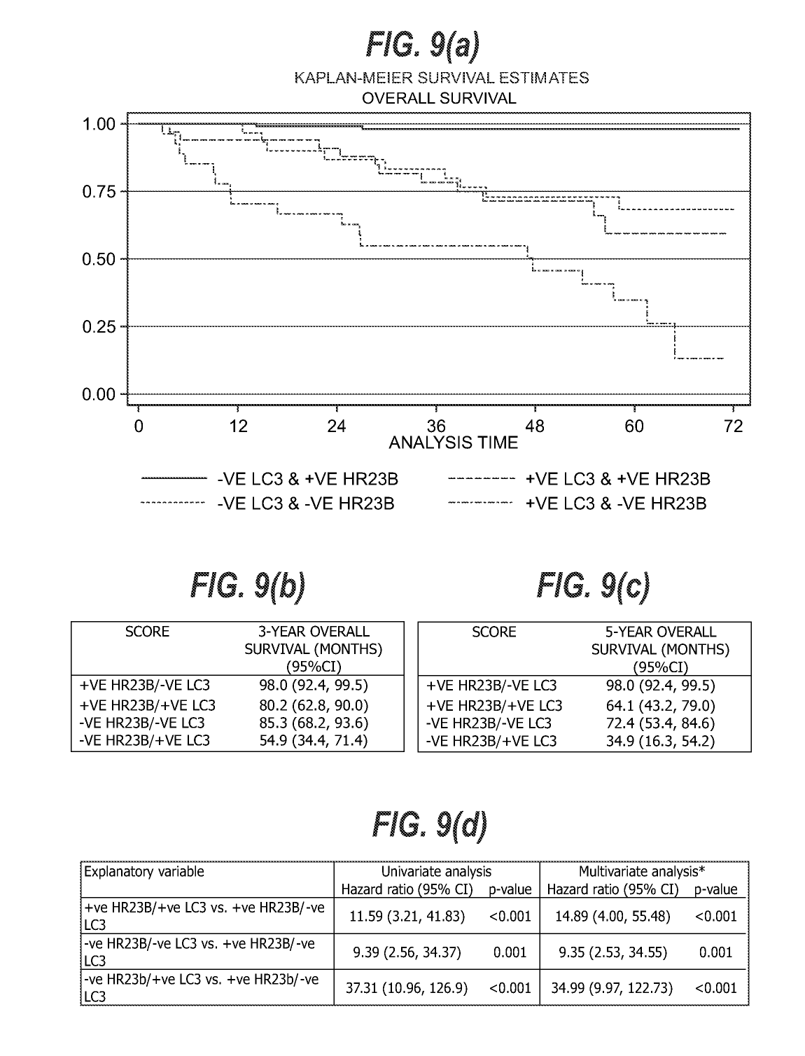

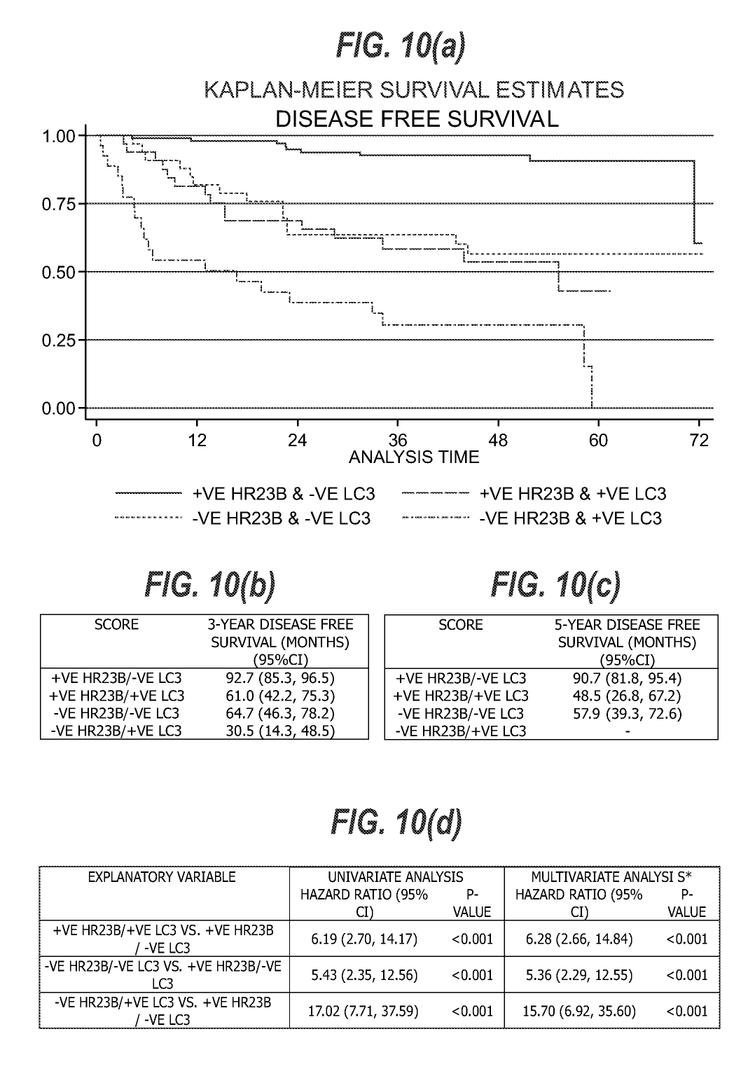

The effect of HDAC inhibitor treatment on the level of markers for autophagy was evaluated in different cell types. The HDAC inhibitor SAHA caused LC3 cleavage as well as the appearance of LAMP1 and cathepsin D in diverse tumour cell lines, for example HUT78, MYLA (CTCL), A2058, A375 (melanoma), HCT116 and HCT15 (CRC; FIG. 1a and FIG. 6a to d). Treatment with other HDAC inhibitors, including valproic acid (VPA), resulted in similar effects (FIG. 1a and FIG. 6a to d), and the increase in autophagy markers coincided with the visual appearance of autophagosomes in cells (for example shown for SAHA; FIG. 1a, v).

Significantly, when cells were co-treated with the inhibitor of autophagy 3-methyladenine (3-MA); (21-23) and SAHA, an enhanced level of apoptosis was apparent (FIG. 1b), suggesting that under these experimental conditions apoptosis and autophagy are separate outcomes of HDAC inhibitor treatment. These results are consistent with previous reports which document the induction of autophagy by HDAC inhibitors (18, 20).

The appearance of autophagocytic markers at increasing SAHA concentration frequently coincided with decreased levels of HR23B (FIG. 1a and FIG. 6a to d), and therefore it is surmised that a relationship might exist between HR23B and autophagy. Since previous studies established that high levels of HR23B sensitise cells to apoptosis in response to HDAC inhibitor treatment (12, 16), studies were conducted to examine in greater detail the influence that different levels of HR23B had on cellular outcome. To pursue this line of investigation, the level of HR23B was altered, either by expressing high levels of ectopic HR23B protein in stable cell lines or depleting endogenous HR23B with siRNA, and thereafter assessed the impact on autophagy.

Reduced levels of LC3 cleavage and enhanced PARP cleavage occurred in cells expressing ectopic HR23B (FIG. 1ci, ii and dii) and decreased levels of HR23B resulted in reduced apoptosis (FIG. 1biii). Increased levels of autophagy (measured as the appearance of cleaved LC3) were also apparent in HR23B siRNA treated cells compared to the control treatment (FIG. 6e). Further, by immunostaining, the autophagosomes apparent in SAHA treated cells were less evident when ectopic HR23B was expressed (FIG. 1d, quantitated in iii).

The role of HR23B was further evaluated by measuring its effect in cell colony formation assays, in the presence and absence of SAHA. The expression of ectopic HR23B (in the stable cell line; +Dox) caused a markedly enhanced growth inhibitory effect of the drug compared to uninduced (−Dox) cells, reflected as a reduction in cell colony formation number (FIG. 1e; quantitated below). Thus, HR23B has the ability to influence the biological outcome of SAHA treatment, specifically the level of apoptotic and autophagocytic cells.

### Example 3

### HR23B Influences the Level of Ubiquitinated Proteins and Proteasome Activity

HR23B binds and shuttles ubiquitinated cargo proteins to the proteasome (13, 14), and proteasome targeting by HR23B is likely to be important for its role as an HDAC inhibitor sensitivity determinant (12, 16). This possibility was evaluated in greater detail by studying the level of global ubiquitination in HDAC inhibitor treated cells, and then the impact of HR23B on ubiquitination and directly on proteasome activity.

To pursue this line of investigation, His-tagged ubiquitin was expressed in transfected cells and the level of ubiquitin-conjugated proteins was assessed by immunoblotting with anti-His antibodies (24) under conditions of drug treatment that causes growth inhibition (12, 16). The global ubiquitination pattern that occurred in SAHA treated (for 20 hr) cells was similar to control untreated cells (FIG. 2a). An increase in ubiquitination was apparent when the ubiquitin proteasome system (UPS) was directly impaired by treating cells with MG132, and cells treated with both SAHA and MG132 had an even greater level of ubiquitination (FIGS. 2a and b, quantitation of ubiquitin signal shown below; ii). Upon the expression of ectopic HR23B (+Dox) in the stable cells and treatment with either agent alone or combined, a higher level of global ubiquitination was apparent when compared to the uninduced (−Dox) cells (FIG. 2a). Thus, a greater amount of ubiquitinated protein was present in cells under conditions of increased HR23B expression. The increased global ubiquitination pattern evident upon expressing HR23B contrasted with the effect of depleting HR23B with siRNA, where a reduced level of ubiquitinated protein was apparent under single or combined drug treatment conditions (FIG. 2b). These results indicate that HR23B impacts on the amount of ubiquitinated protein present in cells, and further that this coincides with the apoptotic effect of HDAC inhibitors (for example FIG. 1cii).

In order to directly measure proteasomal activity under different levels of HR23B expression, the level of GFP^{u} was studied in transfected cells. GFP^{u} is a derivative of GFP protein that is targeted to the proteasome by virtue of the CI1 degron sequence and thus acts as a surrogate and independent measure of proteasomal activity (12, 25). In untreated cells, the GFP^{u} reporter was constitutively degraded, as expected, in contrast to the non-proteasome targeted GFP reporter (FIG. 2c), reflecting GFP^{u} targeting to the proteasome (25). In the presence of an increased level of HR23B (expressed in the stable inducible cell line; +Dox), the GFP^{u} level was higher, contrasting with the non-targeted GFP where the level remained unchanged (FIG. 2c; quantitation of GFP level shown below).

In the converse experiment, depleting HR23B (with siRNA) decreased the level of GFP^{u}, in contrast to the minimal effect on the nontargeted GFP protein which remained unchanged (FIG. 2d), implying that reduced levels of HR23B enhance protein degradation.

Combined with the previous results (FIGS. 2a and b), it is concluded that global ubiquitination and proteasomal activity is influenced by HR23B, and that the high levels of ubiquitination that occur upon increased HR23B expression most likely impair proteasome function (and conversely low levels of ubiquitination enhance proteasome activity). It is, therefore, envisaged that high levels of ubiquitinated substrates saturate the proteasome and that the consequently impaired proteasome activity prevents the degradation of targeted proteins (such as, for example, GFP^{u}), which further provides a mechanism that inhibits cell proliferation.

### Example 4

### Interplay Between HR23B and HDAC6

To gather insight on the mechanisms that influence HR23B activity and thereby impact on the UPS and cellular outcome of HDAC inhibitors. a role for HDAC6 was considered. HDAC6 has been established to control protein turnover and autophagy (26-28). Experiments were conducted to test whether an interaction between HR23B and HDAC6 was apparent and it was found that HDAC6 and HR23B interact in cells (FIG. 3a). A similar interaction was apparent with ectopically expressed proteins (FIGS. 4a and b).

Studies were then conducted to assess whether HDAC6 was able to influence HR23B protein levels by measuring HR23B in cells stably expressing HDAC6 shRNA, where HDAC6 was virtually undetectable (referred to as KD; FIG. 3b). The level of HR23B was markedly higher in the HDAC6 deficient cells compared to the normal control cells (FIG. 3b), contrasting with HR23B RNA which remained similar (FIG. 6f), thus indicating that the impact of HDAC6 was post-transcriptional. Further, an increase in HR23B levels was apparent when the normal A549 cells were transfected with HDAC6 siRNA compared to the control NT siRNA treatment (FIG. 3c; compare HDAC6 to HR23B level). Reduced levels of HR23B were apparent in stable inducible HDAC6 expressing cells upon the expression of HDAC6 (FIG. 3d; compare HDAC6 to HR23B level). Thus, overall, a biochemical relationship exists between HR23B and HDAC6 in which HDAC6 interacts with and negatively regulates the level of HR23B.

Because the level of HR23B is influenced by HDAC6 (FIG. 3b, c and d), and since high levels of HR23B sensitise cells to apoptosis (FIG. 1c), it can be reasoned that the absence of HDAC6 might impact on cell viability in response to HDAC inhibitors. This possibility was evaluated in normal (WT) and HDAC6 deficient (KD) A549 cells. In contrast to WT cells, where a typical low level of apoptosis was apparent and increased apoptosis occurred upon treatment with HDAC inhibitors (about 40-fold), HDAC6 deficient cells had an unusually high basal level of apoptosis (25-fold higher than WT cells) and a lower relative increase in apoptosis (about 2- to 3-fold) occurred upon HDAC inhibitor treatment (FIG. 3e, i and ii; apoptosis measured as the sub-G1 population by flow cytometry).

The level of global ubiquitination was, therefore, evaluated in HDAC6 KD cells compared to their WT counterparts, and it was found that ubiquitination was higher in the KD cells (FIG. 3f, which shows the quantitation of endogenous ubiquitination signal underneath). The higher level of ubiquitination, and coincident increased level of HR23B in the KD cells (FIG. 3f), is compatible with the earlier results describing the higher ubiquitination signal under conditions of increased HR23B expression (FIG. 2a). The enhanced sensitivity to apoptosis upon HDAC inhibitor treatment of the KD A549 cells compared to their WT counterparts (FIG. 3e) is similarly compatible with the role of HR23B as a positive regulator of HDAC inhibitor induced apoptosis (FIG. 1c, d and e). Significantly, enhanced global ubiquitination was also apparent when WT A549 cells were treated with HDAC6 siRNA compared to the NT siRNA control treatment (FIG. 3g). Again, this agrees with the earlier results (FIG. 2a), as depleting HDAC6 caused an increase in HR23B which coincided with higher levels of GFP^{u} but not the untagged GFP version (FIGS. 3c and g). Most importantly, the increased level of HDAC6 (upon induction in the HDAC6 stable cell line; +Dox) and coincidental decrease in HR23B reflected a reduced level of GFP^{u} but not GFP (FIG. 3d). This was the expected outcome, based on the increase in proteasomal activity and protein turnover (leading to lower levels of GFP^{u}), under conditions of increased HDAC6 and the consequent reduced HR23B levels (FIG. 2d).

Furthermore, autophagy was far more pronounced in WT A549 cells, and the level of autophagy markers (LC3 cleavage, LAMP1 and cathepsin D) was reduced in the HDAC6 KD cells compared to the WT counterparts (FIG. 3e, iii). This effect was also apparent when normal A549 cells were treated with HDAC6 siRNA, whereupon the number of autophagocytic cells (measured by LC3 immunostaining) was significantly reduced relative to the control NT siRNA treatment, and contrasted with the number of apoptotic cells (measured by cleaved PARP immunostaining) which increased under the same HDAC6 siRNA treatment conditions (FIGS. 3h and i). Overall, these results establish that HDAC6 is a negative regulator of HR23B protein level, which couples HDAC6 activity (via its interplay with HR23B) to the apoptotic and autophagocytic outcome of treatment with HDAC inhibitors.

### Example 5

### Regulation of HR23B Occurs Independently of HDAC6 Catalytic Activity

To understand in greater detail the mechanism responsible for the regulation of HR23B by HDAC6, particularly the role of its histone deacetylase activity, it was investigated which domains are required for the interaction between HR23B and HDAC6. Using a series of mutants derived from each protein which were expressed ectopically in cells, the HDAC6 BUZ domain was found to be responsible for the interaction with HR23B because the ΔN and BUZ mutant, both containing the BUZ domain, could interact with HR23B whereas BUZ showed a diminished interaction (FIG. 4a, ii and iii); note that the ΔBUZ-HR23B interaction was not completely abolished most likely because of the high level of ectopic ΔBUZ. Conversely, the UbL domain in HR23B was necessary for the interaction with HDAC6 because the ΔUbA mutant, containing only the UbL domain, could interact with HDAC6 whereas ΔUbL could not (FIG. 4b, ii).

As described above (FIG. 3d), in cells stably expressing HDAC6, the induction of HDAC6 coincided with reduced levels of HR23B (FIG. 4ci); a similar effect on HR23B was evident by immunostaining (FIG. 4cii). Moreover, ectopic HDAC6 reduced the level of HR23B in transfected cells (FIG. 4d). Significantly, the BUZ domain was required to down-regulate HR23B because ΔBUZ failed to alter the level of HR23B (FIG. 4d). This contrasts with the expression of the BUZ mutant where down-regulation of HR23B was evident (FIG. 4e). As the BUZ domain is sufficient to affect HR23B, these results equally establish that the catalytic activity of HDAC6 is dispensable for the down-regulation of HR23B. Thus, the HDAC6 BUZ domain is functionally important in regulating HR23B, and therefore the BUZ domain via HR23B impacts on the level of global ubiquitination in cells.

### Example 6

### Role of HDAC6 in the Apoptosis-Autophagy Switch

Given that HDAC6 is a positive regulator of autophagy (28), it appeared possible that HDAC6 may prompt down-regulation of HR23B by an autophagocytic process. The impact of the autophagy inhibitor 3-MA was therefore assessed, but this failed to affect down-regulation of HR23B by HDAC6 (FIG. 5a), thus suggesting that HR23B down-regulation does not occur through an autophagocytic pathway. Similarly, HDAC6 did not mediate its effects on HR23B via the proteasome, because proteasome inhibitors did not affect the HDAC6 mediated down-regulation (FIG. 6g).

Thus, in order to explore the mechanism through which HDAC6 down-regulates HR23B, the possibility was considered that HDAC6 might mediate its effects by interacting with, and perhaps regulating, additional proteins. To address this idea, a proteomics approach was taken. HDAC6 was immunoprecipitated from the induced stable cells (FIG. 5b) and the interacting proteome was analysed. A variety of proteins were found to be immunoprecipitated with HDAC6 compared to the control treatment (FIG. 5c). These proteins were subsequently identified by mass spectrometry (presented as protein clusters in FIG. 5d and each identified protein detailed in Table 1).

### Table 1 Provides Details of HDAC6 Interacting Proteins.

It shows the major proteins interacting with HDAC6 after its overexpression and immunoprecipitation (FIG. 5c). The proteins in the bands of the colloidal blue gel were identified by mass spectrometry. Known and putative contaminants such as keratins were excluded from the list and only proteins were listed with a Mascot score of 40 and with at least two significant peptide matches. Proteins belonging to the same cluster (according to FIG. 5d) are coloured accordingly. The table shows the respective Mascot scores, except column 1 which was manually annotated: column 1 (Band_num) indicates the corresponding band in FIG. 5 c); column 2 (prot_hit_num) the number of the identified protein within this band; column 3 (prot_acc) gives the respective uniprot accession numbers; column 4 (prot_desc) gives the name, alternative names and abbreviations of the proteins; column 5 (prot_score) the mascot score; column 6 (prot_mass) gives the mass of the identified protein.

Some of the proteins had been previously shown to interact with HDAC6, such as β tubulin and its network (29) and the molecular chaperones HSP90 and GRP78/HSPA5 (30). Other components of the HDAC6 interactome that were newly characterised in this study and occurred repeatedly across different mass spectrometry analyses included the chaperonin TCP1 complex (31), DNA repair proteins such as Ku80/XRCC5, PCNA, and MSH2 (32), components of the proteasome such as PSMD1 (26S proteasome regulatory subunit 1) and metabolic enzymes such as PCK1 (phosphoenolpyruvate carboxykinase 1). This suggests that HDAC6 has the potential to influence a number of different protein networks (FIG. 5d). These interactions were similarly evident in the inducible HDAC6 stable cells under immunoprecipitation conditions, for example, between HDAC6 and HSP90, GRP78 and Ku80 (FIGS. 5e and f).

The ability of HDAC6 to bind to interacting partners, like HSP90, suggested an alternative possibility, namely that HDAC6 influences HR23B by targeting this network. Indeed, previous reports have established HDAC6 as an activator of HSP90 chaperone activity (Kovacs et al, 2005). Consequently, the role of HSP90 in the HDAC6-dependent down-regulation of HR23B was evaluated using the HSP90 inhibitor 17-AAG (33). Notably, HDAC6 failed to down-regulate HR23B when HSP90 chaperone activity was inhibited with 17-AAG (FIG. 5g). Moreover, the inhibition of apoptosis that occurred upon the expression of HDAC6 (caspase 3 cleavage; FIG. 5g) was overcome when HDAC6-induced cells were treated with 17-AAG, which therefore paralleled the increase in HR23B levels (FIG. 5g). Importantly, although inhibiting HDAC6 catalytic activity (with tubastatin) did not impact on the HDAC6-dependent down-regulation of HR23B, it did prevent the increase in HR23B upon 17-AAG treatment (FIG. 5g), confirming the catalytic independent role of HDAC6 in the down-regulation of HR23B, but equally suggesting a role for HDAC6 enzyme activity in the reactivation of HR23B upon treatment with 17-AAG.

The results indicate therefore that HDAC6 is a negative regulator of HDAC inhibitor-induced apoptosis through its ability to down-regulate HR23B, and further that HDAC6 prompts the down-regulation of HR23B through the HSP90 chaperone network.

### Example 7

### Prognostic Significance of HR23B

Autophagy is becoming increasingly recognised as a mechanism that cancer cells use to survive under adverse conditions, for example, chemotherapy (Kroemer, G., Marino, G. & Levine, B. Autophagy and the integrated stress response. Mol Cell 40, 280-293 (2010)). It is reasoned therefore that tumours exhibiting different levels of HR23B and LC3 may differ in their clinical history and prognosis, reflecting the detrimental influence that surviving autophagocytic tumour cells might have on therapeutic response and clinical outcome. It was therefore of interest to relate the expression of HR23B and LC3 to clinical history, and then consider its prognostic significance.

Studies of HR23B and LC3 levels were carried out in a sample of 204 patients from the VICTOR trial (Midgley, R. S., et al. Phase III randomized trial assessing rofecoxib in the adjuvant setting of colorectal cancer: final results of the VICTOR trial. J Clin Oncol 28, 4575-4580 (2010) where clinical history had been followed up and annotated over a 5 year period. In total about 1200 biopsies were screened (samples were in triplicate with matched normal colon biopsies), and given a score reflecting the overall staining characteristics of HR23B and LC3, which was then related to clinical disease (specifically disease free survival (DFS)). FIGS. 7a and 7b show an example of the high/low expression of HR23B and LC3 in situ in human tumours. The graphs in FIGS. 7c and 7d show the prognosis of disease which has this type of expression pattern. Tumours expressing high levels of HR23B had improved DFS compared to low expressors (FIG. 7c). Conversely, high LC3 levels correlated with reduced DFS compared to tumours expressing low LC3 levels (FIG. 7d), suggesting that the presence of autophagocytic cells is a poor prognostic indicator. Remarkably, when both data sets were combined, the difference between DFS in high HR23B/low LC3 and low HR23B/high LC3 was even greater (1856 days to 1037 days; data not shown). By analogy with the results derived from the cell line studies, the correlation between high HR23B and improved DFS most likely reflects the propensity of tumour cells for apoptosis. Conversely, tumours where low HR23B coincides with high LC3 and poor prognosis may reflect disease where autophagocytic mechanisms prevail. These results suggest therefore that the level of HR23B and LC3 provides important prognostic information. Significantly, the studies shown in FIG. 7 were conducted in a variety of colorectal samples from HDAC inhibitor naïve patients, and therefore demonstrate that HR23B and LC3 levels are prognostic markers for autophagy for any drug, not only for HDAC inhibitors.

### Example 8

### Prognostic Significance of HR23B and LC3

The VICTOR trial was a phase III randomised, placebo-controlled double-blind trial of rofecoxib in patients after potentially-curative surgery and completion of adjuvant therapy for stage II/III colorectal cancer. This study is registered with ISRCTN, number 98278138. VICTOR stopped recruiting early due to withdrawal of rofecoxib because of safety issues but follow-up continued regularly for a further 5 years.

Originally it was planned to recruit 7000 patients to the VICTOR trial. At the time of rofecoxib withdrawal, 2434 patients had been recruited at 151 hospitals in the UK, although 107 had not yet started treatment when the drug was withdrawn. The treated population therefore comprised 1167 rofecoxib and 1160 placebo patients. These patients were followed up regularly for survival and recurrence. Median follow-up was 4.84 and 4.85 years (rofecoxib versus placebo) with 241 versus 246 deaths and 297 versus 329 recurrences.

Baseline characteristics for all VICTOR participants, split by those who provided consent for tissue sample research and those who did not, and those with evaluable samples, are presented in the table below.

| | | VICTOR | | Patients without | | Patients with | | Patients to | |
|---|---|---|---|---|---|---|---|---|---|
| | | patients | | Samples | | samples | | be analysed | |
| | | (N = 2434) | | (N = 1428) | | (N = 1006) | | (N = 204) | |
| | | N (%) | | N (%) | | N (%) | | N (%) | |
| Age^{†} | Age <50 | 181 | (7.44) | 109 | (7.63) | 72 | (7.16) | 14 | (6.86) |
| | Age 50-59 | 590 | (24.24) | 349 | (24.44) | 241 | (23.96) | 50 | (24.51) |
| | Age 60-69 | 940 | (38.62) | 557 | (39.01) | 383 | (38.07) | 64 | (31.37) |
| | Age 70+ | 723 | (29.7) | 413 | (28.92) | 310 | (30.82) | 76 | (37.25) |
| Cancer Site^{†} | Colon | 1592 | (65.41) | 914 | (64.01) | 678 | (67.40) | 138 | (67.65) |
| | Junction | 181 | (7.44) | 102 | (7.14) | 79 | (7.85) | 15 | (7.35) |
| | Rectum | 661 | (27.16) | 412 | (28.85) | 249 | (24.75) | 51 | (25.00) |
| Stage^{†} | Stage II | 1159 | (47.62) | 667 | (46.71) | 492 | (48.91) | 64 | (31.37) |
| | Stage III | 1275 | (52.38) | 761 | (53.29) | 514 | (51.09) | 140 | (68.63) |
| Chemotherapy^{†} | None | 1580 | (64.9) | 943 | (66.04) | 369 | (36.68) | 58 | (28.43 |
| | Yes | 854 | (35.1) | 485 | (33.96) | 637 | (63.32) | 146 | (71.57) |
| Radiotherapy | Yes | 299 | (12.3) | 191 | (13.38) | 108 | (10.74) | 18 | (8.82) |
| | None | 2135 | (87.7) | 1237 | (86.62) | 898 | (89.26) | 186 | (91.18) |
| Gender | Male | 1560 | (64.09) | 650 | (64.61) | 910 | (63.73) | 129 | (63.24) |
| | Female | 874 | (35.91) | 656 | (35.39) | 518 | (36.27) | 75 | (36.76) |
| Age | Age (mean | 63.78 | (9.68) | 63.52 | (9.54) | 64.15 | (9.87) | 64.91 | (10.14) |
| | (SD)) | | | | | | | | |
| Treatment | Placebo | 1217 | (50.0) | 722 | (50.6) | 706 | (49.4) | 97 | (47.55) |
| | Vioxx | 1217 | (50.0) | 495 | (49.2) | 511 | (50.8) | 107 | (52.45) |

^{†}Minimisation factors for VICTOR trial

The survival characteristics for VICTOR participants, overall and split between patients who provided samples for research and those who did not, and those with evaluable samples differences, in each of these cohorts and overall are shown in the table below.

| | VICTOR | | Patients without | | Patients with | | Patients to | |
|---|---|---|---|---|---|---|---|---|
| | Patients | | samples | | samples | | be analysed | |
| | (N = 2434) | | (N = 1428) | | (N = 1006) | | (N = 204) | |
| Deaths (N (%) | 487 | (20.0) | 294 | (20.59) | 193 | (19.18) | 42 | (20.59) |
| Recurrences/deaths | 704 | (28.92) | 427 | (29.90) | 277 | (27.53) | 59 | (28.92) |
| (any cause) | | | | | | | | |
| Overall Survival | 87.3 | (85.8, 88.6) | 86.6 | (84.7, 88.3) | 88.1 | (85.9, 90.0) | 87.0 | (81.4, 90.9) |
| (3-year % (95% CI)) | | | | | | | | |
| Overall Survival | 78.2 | (76.3, 78) | 77.0 | (74.5, 79.4) | 79.7 | (76.8, 82.2) | 79.0 | (72.1, 84.4) |
| (5-year % (95% CI)) | | | | | | | | |
| Disease Free Survival | 74.6 | (72.7, 76.3) | 73.5 | (71.0, 75.8) | 76.1 | (73.2, 78.7) | 74.0 | (67.2, 79.6) |
| (3-year % (95% CI)) | | | | | | | | |
| Disease Free Survival | 66.5 | (64.3, 68.7) | 65.3 | (62.4, 68.1) | 68.2 | (64.5, 71.5) | 66.4 | (57.7, 73.7) |
| (5-year % (95% CI)) | | | | | | | | |

Overall survival is defined as the time from randomisation until death from any cause. Patients not reported as dead were censored at their last known date alive.

Disease free survival, a secondary endpoint, is defined as time from randomisation until recurrence or death from any cause. Patients who were alive and recurrence-free were censored at their last known recurrence-free date.

Patients with positive HR23B and negative LC3 were compared with patients with negative HR23B and positive LC3. All patients with positive HR23B and negative LC3 or negative HR23B and positive LC3 staining were included in the analysis. HR23B was analysed as positive and negative with positive staining being a score of 5-7 and negative staining being a score of 0-4. LC3 was analysed as positive and negative with positive staining being >5% of tumour cells and negative staining being ≦5%. The table below provides the HR23B and LC3 score frequencies in the patients analysed.

| | Negative HR23B | Positive HR23B | Total |
|---|---|---|---|
| Negative LC3 | 34 | 107 | 141 |
| Positive LC3 | 27 | 36 | 63 |
| Total | 61 | 143 | 204 |

Kaplan Meier plots of the overall survival and disease free survival are provided in FIG. 9a and FIG. 10a, respectively. FIGS. 9b, 9c, 10b and 10c show the 3-year and/or 5-year overall and disease free survival times with 95% confidence intervals, overall and by expression, defined as the survival probability at 36 months or 60 months, from the Kaplan Meier graph taking into account both events and censored observations. A Cox proportional hazards model was fitted using biomarker expression, treatment, and clinical factors including gender, site (colon/rectum), stage (II/III), age and prior treatment. Hazard ratios and 95% confidence intervals are given in The data show that patients having tumours that are negative for HR23B and negative for LC3, positive for HR23B and positive for LC3, and negative for HR23B and positive for LC3, all have a worse overall survival and disease free survival when compared to patients with tumours that are positive for HR23B and negative for LC3. Patients with tumours that are negative for HR23B and positive for LC3 have the worst prognosis.

In conclusion, the Examples highlight HR23B as a key regulator of the cellular response to HDAC inhibitors. Given the potential importance of apoptosis and autophagy in cancer and the outcome of disease progression, it remains possible that HR23B, perhaps in conjunction with HDAC6 and HSP90, is a biomarker signature which has the ability to differentiate between tumours that have the propensity to enter apoptosis verses autophagy (FIG. 5h). This could provide a powerful step forward in developing approaches that aim to align the most effective cancer treatment with clinical disease.

TABLE 1

| Band_nu | Prot_hit_nu | Prot_acc | Prot_desc | Prot_sco | Prot_mas |
|---|---|---|---|---|---|
| I | I | P27708 | CAD protein | 690 | 245167 |
| 2 | 4 | P68363 | Tubulin alpha-IB chain | 246 | 50804 |
| 2 | 9 | Q9NXG0 | Centlein | 49 | 162131 |
| 2 | 25 | Q9HC77 | Centromere protein J | 46 | 154103 |
| 3 | 3 | Q9NY65 | Tubulin alpha-8 chain Brefeldin A-resistance | 134 | 50746 |
| | | | guanine | | |
| 3 | 22 | Q92538 | exchange factor 1 | 56 | 208367 |
| 3 | 28 | Q8IVL1 | Neuron navigator 2 | 47 | 269314 |
| 4 | 1 | Q9UBN7 | Histone deacetylase 6 | 7691 | 132932 |
| 5 | 6 | P55060 | Exportin-2 | 165 | 111145 |
| 5 | 7 | P43246 | DNA mismatch repair protein Msh2 | 119 | 105418 |
| 5 | 9 | P34932 | Heat shock 70 kDa protein 4 | 83 | 95127 |
| 5 | 20 | Q99460 | 26S proteasome non-ATPase regulatory | 58 | 106795 |
| | | | subunit 1 | | |
| 5 | 23 | Q96R11 | Bile acid receptorHRR-1 | 54 | 56847 |
| 5 | 43 | Q14697 | Neutral alpha-glucosidase AB | 46 | 107263 |
| 6 | 1 | P08238 | Heat shock protein HSP 90-beta | 1608 | 83554 |
| 6 | 2 | Q9Y263 | Phospholipase A-2-activating protein | 1096 | 88641 |
| 6 | 3 | P07900 | Heat shock protein HSP 90-alpha | 817 | 85006 |
| 6 | 6 | Q01813 | 6-phosphofructokinase type C | 327 | 86454 |
| 6 | 7 | PI3010 | X-ray repair cross-complementing protein 5 | 267 | 83222 |
| 6 | 8 | PI 7858 | 6-phosphofructokinase, liver type | 240 | 85762 |
| 6 | 10 | P14625 | Endoplasmin | 190 | 92696 |
| 6 | 12 | P33993 | DNA replication licensing factor MCM7 | 183 | 81884 |
| 6 | 16 | Q92499 | ATP-dependent RNA helicase DDX1 | 134 | 83349 |
| 7 | 1 | PI 1021 | 78 kDa glucose-regulated protein | 656 | 72402 |
| 7 | 2 | PI 1142 | Heat shock cognate 71 kDa protein | 199 | 71082 |
| 7 | 4 | P54652 | Heat shock-related 70 kDa protein 2 | 170 | 70263 |
| 7 | 5 | P34931 | Heat shock 70 kDa protein 1 -like | 156 | 70730 |
| 7 | 6 | PI 7066 | Heat shock 70 kDa protein 6 | 156 | 71440 |
| 8 | 1 | P48643 | T-complex protein 1 subunit epsilon | 343 | 60089 |
| 8 | 2 | P14618 | Pyruvate kinase isozymes M1/M2 | 250 | 58470 |
| 8 | 4 | P49368 | T-complex protein 1 subunit gamma | 214 | 61066 |
| 8 | 5 | PI 7987 | T-complex protein 1 subunit alpha | 212 | 60819 |
| 8 | 6 | Q99832 | T-complex protein 1 subunit eta | 203 | 59842 |
| 9 | 1 | P07437 | Tubulin beta-5 chain | 140 | 50095 |
| 9 | 2 | Q9BQE3 | Tubulin alpha- 1C chain | 136 | 50548 |
| 9 | 3 | P68371 | Tubulin beta-4B chain | 132 | 50255 |
| 9 | 4 | QI3509 | Tubulin beta-3 chain | 109 | 50856 |
| 9 | 5 | P04350 | Tubulin beta-4A chain | 109 | 50010 |
| 9 | 5 | Q71U36 | Tubulin alpha-1A chain | 93 | 50788 |
| 10 | 3 | P00558 | Phosphoglycerate kinase 1 | 163 | 44985 |
| 10 | 4 | P31689 | DnaJ homolog subfamily A member 1 | 150 | 45581 |
| 10 | 5 | P62195 | 26S protease regulatory subunit 8 | 136 | 45768 |
| 10 | 7 | Q13148 | TAR DNA-binding protein 43 | 118 | 45053 |
| 10 | 19 | Q9BRX2 | Protein pelota homolog | 59 | 43788 |
| 11 | 5 | PI2004 | Proliferating cell nuclear antigen | 112 | 29092 |
| 11 | 10 | P51956 | Serine/threonine-protein kinase Nek3 | 69 | 58181 |
| 11 | 11 | P62753 | 40S ribosomal protein S6 | 66 | 28834 |
| 11 | 21 | Q9NQR1 | N-lysine methyltransferase SETD8 | 50 | 43376 |
| 12 | 4 | 013315 | Serine-protein kinase ATM | 55 | 355564 |

### REFERENCES

1. Inche A G, La Thangue N B. Chromatin control and cancer-drug discovery: realizing the promise. Drug Discov Today 2006 February; 11(3-4): 97-109.
2. Bolden J E, Peart M J, Johnstone R W. Anticancer activities of histonedeacetylase inhibitors. Nat Rev Drug Discov 2006 September; 5(9): 769-784.
3. Minucci S, Pelicci P G. Histone deacetylase inhibitors and the promise of epigenetic (and more) treatments for cancer. Nat Rev Cancer 2006 January; 6(1):38-51.
4. Choudhary C, Kumar C, Gnad F, Nielsen M L, Rehman M, Walther T C, et al. Lysine acetylation targets protein complexes and co-regulates major cellular functions. Science 2009 Aug. 14; 325(5942): 834-840.
5. Marks P, Rifkind R A, Richon V M, Breslow R, Miller T, Kelly W K. Histone deacetylases and cancer: causes and therapies. Nat Rev Cancer 2001 December; 1(3):194-202.
6. Marks P A, Breslow R. Dimethyl sulfoxide to vorinostat: development of this histone deacetylase inhibitor as an anticancer drug. Nat Biotechnol 2007 January; 25(1): 84-90.
7. Richon V M, Garcia-Vargas J, Hardwick J S. Development of vorinostat: current applications and future perspectives for cancer therapy. Cancer Lett 2009 Aug. 8; 280(2): 201-210.
8. Duvic M, Vu J. Vorinostat: a new oral histone deacetylase inhibitor approved for cutaneous T-cell lymphoma. Expert Opin Investig Drugs 2007 July; 16(7):1111-1120.
9. Khan O, La Thangue N B. Drug Insight: histone deacetylase inhibitor-based therapies for cutaneous T-cell lymphomas. Nat Clin Pract Oncol 2008 December; 5(12): 714-726.
10. Mann B S, Johnson J R, Cohen M H, Justice R, Pazdur R. FDA approval summary: vorinostat for treatment of advanced primary cutaneous T-cell lymphoma. Oncologist 2007 October; 12(10): 1247-1252.
11. Stimson L, La Thangue N B. Biomarkers for predicting clinical responses to HDAC inhibitors. Cancer Lett 2009 Aug. 8; 280(2): 177-183.
12. Fotheringham S, Epping M T, Stimson L, Khan 0, Wood V, Pezzella F, et al. Genome-wide loss-of-function screen reveals an important role for the proteasome in HDAC inhibitor-induced apoptosis. Cancer Cell 2009 Jan. 6; 15(1): 57-66.
13. Chen L, Shinde U, Ortolan T G, Madura K. Ubiquitin-associated (UBA) domains in Rad23 bind ubiquitin and promote inhibition of multi-ubiquitin chain assembly. EMBO Rep 2001 October; 2(10):933-938.
14. Chen L, Madura K. Rad23 promotes the targeting of proteolytic substrates to the proteasome. Mol Cell Biol 2002 July; 22(13): 4902-4913.
15. Schauber C, Chen L, Tongaonkar P, Vega I, Lambertson D, Potts W, et al. Rad23 links DNA repair to the ubiquitin/proteasome pathway. Nature 1998 Feb. 12; 391(6668): 715-718.
16. Khan 0, Fotheringham S, Wood V, Stimson L, Zhang C, Pezzella F, et al. HR23B is a biomarker for tumor sensitivity to HDAC inhibitor-based therapy. Proc Natl Acad Sci USA 2010 Apr. 6; 107(14): 6532-6537.
17. Duvic M, Talpur R, Ni X, Zhang C, Hazarika P, Kelly C, et al. Phase 2 trial of oral vorinostat (suberoylanilide hydroxamic acid, SAHA) for refractory cutaneous T-cell lymphoma (CTCL). Blood 2007 Jan. 1; 109(1): 31-39.
18. Shao Y, Gao Z, Marks P A, Jiang X. Apoptotic and autophagic cell death induced by histone deacetylase inhibitors. Proc Natl Aced Sci USA 2004 Dec. 28; 101(52): 18030-18035.
19. Stimson L, Wood V, Khan 0, Fotheringham S, La Thangue N B. HDAC inhibitor-based therapies and haematological malignancy. Ann Oncol 2009 August; 20(8): 1293-1302.
20. Gammoh N, Lam D, Puente C, Ganley I, Marks P A, Jiang X. Role of autophagy in histone deacetylase inhibitor-induced apoptotic and nonapoptotic cell death. Proc Natl Acad Sci USA 2012 Apr. 24; 109(17): 6561-6565.
21. Yamamoto A, Tagawa Y, Yoshimori T, Moriyama Y, Masaki R, Tashiro Y. Bafilomycin A1 prevents maturation of autophagic vacuoles by inhibiting fusion between autophagosomes and lysosomes in rat hepatoma cell line, H-4-II-E cells. Cell Struct Funct 1998 February; 23(1): 33-42.
22. Seglen P O, Gordon P B. 3-Methyladenine: specific inhibitor of autophagic/lysosomal protein degradation in isolated rat hepatocytes. Proc Natl Acad Sci USA 1982 March; 79(6): 1889-1892.
23. Petiot A, Ogier-Denis E, Blommaart E F, Meijer A J, Codogno P. Distinct classes of phosphatidylinositol 3′-kinases are involved in signaling pathways that control macroautophagy in H T-29 cells. J Biol Chem 2000 Jan. 14; 275(2): 992-998.
24. Camus S, Menendez S, Fernandes K, Kua N, Liu G, Xirodimas D P, et al. The p53 isoforms are differentially modified by Mdm2. Cell Cycle 2012 Apr. 15; 11(8): 1646-1655.
25. Bence N F, Bennett E J, Kopito R R. Application and analysis of the GFPu family of ubiquitin-proteasome system reporters. Methods Enzymol 2005; 399: 481-490.
26. Hubbert C, Guardiola A, Shao R, Kawaguchi Y, Ito A, Nixon A, et al. HDAC6 is a microtubule-associated deacetylase. Nature 2002 May 23; 417(6887): 455-458.
27. Pandey U B, Nie Z, Batlevi Y, McCray B A, Ritson G P, Nedelsky N B, et al. HDAC6 rescues neurodegeneration and provides an essential link between autophagy and the UPS. Nature 2007 Jun. 14; 447(7146): 859-863.
28. Lee J Y, Koga H, Kawaguchi Y, Tang W, Wong E, Gao Y S, et al. HDAC6 controls autophagosome maturation essential for ubiquitin-selective quality-control autophagy. EMBO J 2010 Mar. 3; 29(5): 969-980.
29. Valenzuela-Fernandez A, Cabrero J R, Serrador J M, Sanchez-Madrid F.HDAC6: a key regulator of cytoskeleton, cell migration and cell-cell interactions. Trends Cell Biol 2008 June; 18(6): 291-297.
30. Kovacs J J, Murphy P J, Gaillard S, Zhao X, Wu J T, Nicchitta C V, et al. HDAC6 regulates Hsp90 acetylation and chaperone-dependent activation of glucocorticoid receptor. Mol Cell 2005 May 27; 18(5): 601-607.
31. Posokhova E, Song H, Belcastro M, Higgins L, Bigley L R, Michaud N A, et al. Disruption of the chaperonin containing TCP-1 function affects protein networks essential for rod outer segment morphogenesis and survival. Mol Cell Proteomics 2011 January; 10(1): M110 000570.
32. Lisby M, Barlow J H, Burgess R C, Rothstein R. Choreography of the DNA damage response: spatiotemporal relationships among checkpoint and repair proteins. Cell 2004 Sep. 17; 118(6): 699-713.
33. Trepel J, Mollapour M, Giaccone G, Neckers L. Targeting the dynamic HSP90 complex in cancer. Nat Rev Cancer 2010 August; 10(8): 537-549.
34. Kroemer G, Marino G, Levine B. Autophagy and the integrated stress response. Mol Cell 2010 Oct. 22; 40(2): 280-293.
35. Levine B, Kroemer G. Autophagy in the pathogenesis of disease. Cell 2008 Jan. 11; 132(1): 27-42.
36. Kimmelman A C. The dynamic nature of autophagy in cancer. Genes Dev 2011, October 1; 25(19): 1999-2010.
37. Degenhardt K, Mathew R, Beaudoin B, Bray K, Anderson D, Chen G, et al. Autophagy promotes tumor cell survival and restricts necrosis, inflammation, and tumorigenesis. Cancer Cell 2006 July; 10(1): 51-64.
38. Mathew R, Karantza-Wadsworth V, White E. Role of autophagy in cancer. Nat Rev Cancer 2007 December; 7(12): 961-967.
39. Degtyarev M, De Maziere A, Orr C, Lin J, Lee B B, Tien J Y, et al. Aktinhibition promotes autophagy and sensitizes PTEN-null tumors tolysosomotropic agents. J Cell Biol 2008 Oct. 6; 183(1): 101-116.
40. Dang C V. Antimalarial therapy prevents Myc-induced lymphoma. J Clin Invest 2008 January; 118(1): 15-17.
41. Gupta A, Roy S, Lazar A J, Wang W L, McAuliffe J C, Reynoso D, et al. Autophagy inhibition and antimalarials promote cell death in gastrointestinal stromal tumor (GIST). Proc Natl Acad Sci USA 2010 Aug. 10; 107(32): 14333-14338.
42. Kraft C, Peter M, Hofmann K. Selective autophagy: ubiquitin-mediated recognition and beyond. Nat Cell Biol 2010 September; 12(9): 836-841.
43. Voorhees P M, Orlowski R Z. The proteasome and proteasome inhibitors in cancer therapy. Annu Rev Pharmacol Toxicol 2006; 46: 189-213.
44. Boyault C, Gilquin B, Zhang Y, Rybin V, Garman E, Meyer-Klaucke W, et al. HDAC6-p97/VCP controlled polyubiquitin chain turnover. EMBO J 2006 Jul. 26; 25(14): 3357-3366.
45. Moscat J, Diaz-Meco M T. p62 at the crossroads of autophagy, apoptosis, and cancer. Cell 2009 Jun. 12; 137(6): 1001-1004.
46. Korolchuk V I, Menzies F M, Rubinsztein D C. Mechanisms of cross-talk between the ubiquitin-proteasome and autophagy-lysosome systems. FEBS Lett 2010 Apr. 2; 584(7): 1393-1398.
47. Dikic I, Johansen T, Kirkin V. Selective autophagy in cancer development and therapy. Cancer Res 2010 May 1; 70(9): 3431-3434.
48. Scroggins B T, Robzyk K, Wang D, Marcu M G, Tsutsumi S, Beebe K, et al. An acetylation site in the middle domain of Hsp90 regulates chaperone function. Mol Cell 2007 Jan. 12; 25(1): 151-159.
49. Kawaguchi Y, Kovacs J J, McLaurin A, Vance J M, Ito A, Yao T P. The deacetylase HDAC6 regulates aggresome formation and cell viability in response to misfolded protein stress. Cell 2003 Dec. 12; 115(6): 727-738.
50. Jensen L J, Kuhn M, Stark M, Chaffron S, Creevey C, Muller J, et al. STRING8—a global view on proteins and their functional interactions in 630 organisms. Nucleic Acids Res 2009 January; 37(Database issue): D412-416.
51. Shevchenko A, Tomas H, Havlis J, Olsen J V, Mann M. In-gel digestion for mass spectrometric characterization of proteins and proteomes. Nat Protoc 2006; 1(6): 2856-2860.
52. David Y, Ternette N, Edelmann M J, Ziv T, Gayer B, Sertchook R, et al. E3 ligases determine ubiquitination site and conjugate type by enforcing specificity on E2 enzymes. J Biol Chem 2011 Dec. 23; 286(51): 44104-44115.
53. Bastian M, Heymann S, Jacomy M. Gephi: an open source software for exploring and manipulating networks. International AAAI Conference on Weblogs and Social Media 2009.

1. A method for determining the susceptibility of a cell or a patient of interest to entering an autophagocytic state upon treatment with a drug, comprising determining the level of HR23B in the cell or patient.

2. A method according to claim 1, which further comprises determining the level of LC3 in the cell or patient.

3. A method according to claim 1 or claim 2, wherein a low level of HR23B, optionally combined with a high level of LC3, is indicative of the cell or patient being susceptible to entering an autophagocytic state.

4. A method for determining the susceptibility of a cell or a patient of interest to treatment with a drug comprising determining the level of HR23B in combination with the level of one or more of HDAC6, LC3 and HSP90 in the cell or patient.

5. A method according to claim 4, which further comprises determining that the cell or patient has a high level of HR23B and a low level of one or more of HDAC6, LC3 and HSP90 and is therefore susceptible to treatment with the drug.

6. A method according to claim 4, which further comprises determining that the cell or patient has a low level of HR23B and a high level of one or more of HDAC6, LC3 and HSP90 and is therefore not susceptible to treatment with the drug.

7. A method according to any one of claims 4 to 6, for determining the susceptibility of a cell or a patient of interest to treatment with an HDAC inhibitor comprising determining the levels of HR23B and HDAC6 in the cell or patient.

8. The method of claim 7, which further comprises determining that the cell or patient has a high level of HR23B and a low level of HDAC6 and is therefore susceptible to treatment with an HDAC inhibitor.

9. The method of claim 7, which further comprises determining that the cell or patient has a low level of HR23B and a high level of HDAC6 and is therefore not susceptible to treatment with an HDAC inhibitor.

10. The method of claim 4 or claim 5, comprising determining that the cell or patient has a high level of HR23B and a low level of LC3 and is therefore susceptible to treatment with the drug.

11. The method of claim 4 or claim 6, comprising determining that the cell or patient has a low level of HR23B and a high level of LC3 and is therefore not susceptible to treatment with the drug.

12. A method for determining the susceptibility of a cell or a patient of interest to treatment with an HDAC inhibitor comprising determining the level of HSP90 in the cell or patient.

13. The method of any one of claim 4 to 8, 10 or 12, which further comprises determining that the cell or patient has a low level of HSP90 and is therefore susceptible to treatment with an HDAC inhibitor.

14. The method of claim 13, which further comprises determining that the cell or patient has a high level of HR23B and is therefore susceptible to treatment with an HDAC inhibitor.

15. The method of any one of claims 4 to 14, which further comprises administering the drug to the cell or patient if the cell or patient has been found to be susceptible to treatment with the drug.

16. The method of any one of claims 4 to 14, which further comprises administering the drug to the cell or patient if the cell or patient has been found not to have a low level of HR23B and a high level of HDAC6.

17. The method of any one of the preceding claims, wherein the cell is present in a sample that has been obtained from a patient of interest.

18. The method of any one of the preceding claims, wherein the cell is a population of cells.

19. The method of any one of the preceding claims, wherein the cell is a cancer cell, such as a tumour cell.

20. The method of any one of the preceding claims, wherein the patient of interest has cancer.

21. The method of any one of the preceding claims, which further comprises determining the susceptibility of a disease from which the cell is derived to treatment with the drug.

22. The method of any one of the preceding claims, which comprises determining the levels of any one or more of HR23B, HDAC6, HSP90 and LC3 using immunostaining.

23. HR23B in combination with one or more of HDAC6, LC3 and HSP90 for use as biomarkers for determining the susceptibility of a cell or a patient of interest to treatment with a drug.

24. HSP90 for use as a biomarker for determining the susceptibility of a cell or a patient of interest to treatment with a drug.

25. A kit for determining the level of HR23B in combination with the level of one or more of HDAC6, HSP90 and LC3 in a cell or patient of interest, which comprises an anti-HR23B antibody and one or more of an anti-HDAC6 antibody, an anti-HSP90 antibody and an anti-LC3 antibody, optionally in combination with a label for visualising the antibodies.

26. A method of treating a disease or other condition with a drug, which comprises administering the drug to a cell or patient who has been identified as not having a low level of HR23B.

27. A method according to claim 26, wherein the cell or patient has been identified as not having a high level of LC3.

28. A method of treating a disease or other condition with a drug, which comprises administering the drug to a cell or patient who has been identified as having a high level of HR23B in combination with a low level of one or more of HDAC6, HSP90 and LC3 or as not having a low level of HR23B in combination with a high level of one or more of HDAC6, HSP90 and LC3.

29. A method according to claim 28, which comprises administering the drug to a cell or patient who has been identified as having a high level of HR23B and a low level of HDAC6 or as not having a low level of HR23B and a high level of HDAC6.

30. A drug for use in treating a disease or other condition in a cell or patient of interest who has been identified as having a high level of HR23B in combination with a low level of one or more of HDAC6, HSP90 and LC3 or as not having a low level of HR23B in combination with a high level of one or more of HDAC6, HSP90 and LC3.

31. A drug for use according to claim 30, wherein the cell or patient of interest has been identified as having a high level of HR23B and a low level of HDAC6 or as not having a low level of HR23B and a high level of HDAC6.

32. A method of treating a patient having a disease or other condition, wherein the disease or other condition has been determined to have a level of HR23B in combination with a level of one or more of HDAC6, HSP90 and LC3 that indicates that the disease is susceptible to treatment with a drug, wherein the treating comprises administering the drug to the patient.

33. A method of treating a patient having a disease or other condition, wherein the disease or other condition has been determined not to have a low level of HR23B, wherein the treating comprises administering the drug to the patient.

34. A method of increasing the susceptibility of a cell or patient of interest to treatment with a drug comprising increasing the level of HR23B and/or decreasing the level of HDAC6 and/or inactivating or decreasing the level of HSP90 and/or decreasing the level of LC3 in the cell or patient.

35. A method of treating a disease or other condition with a drug, which comprises administering the drug to a cell or patient wherein the treating comprises administering the drug in combination with an HR23B-increasing agent and/or an HDAC6-decreasing agent and/or an HSP90 inhibitor and/or an LC3-decreasing agent, either simultaneously, separately or sequentially.

36. A drug for use in treating a disease or other condition in a cell or patient of interest wherein the treating comprises administering the drugin combination with an HR23B-increasing agent and/or an HDAC6-decreasing agent and/or an HSP90 inhibitor and/or an LC3-decreasing agent, either simultaneously, separately or sequentially.

37. A method according to claim 35 or a drug according to claim 36, wherein the treating comprises administering the HR23B-increasing agent and/or the HDAC6-decreasing agent and/or the HSP90 inhibitor and/or the LC3-decreasing agent before the drug.

38. An HR23B-increasing agent for use in treating a disease or other condition in a cell or patient of interest, wherein the treating comprises administering the HR23B-increasing agent simultaneously, separately or sequentially with a drug and optionally with an HDAC6-decreasing agent and/or with an HSP90 inhibitor and/or with an LC3-decreasing agent.

39. An HDAC6-decreasing agent for use in treating a disease or other condition in a cell of patient of interest, wherein the treating comprises administering the HDAC6-decreasing agent simultaneously, separately or sequentially with a drug and optionally with an HR23B-increasing agent and/or an HSP90 inhibitor and/or an LC3-decreasing agent.

40. An HSP90 inhibitor for use in treating a disease or other condition in a cell or patient of interest, wherein the treating comprises administering the HSP90 inhibitor simultaneously, separately or sequentially with a drug and optionally with an HR23B-increasing agent and/or an HDAC6-decreasing agent and/or an LC3-decreasing agent.

41. An LC3-decreasing agent for use in treating a disease or other condition in a cell or patient of interest, wherein the treating comprises administering the LC3-decreasing agent simultaneously, separately or sequentially with a drug and optionally with an HR23B-increasing agent and/or an HDAC6-decreasing agent and/or an HSP90 inhibitor.

42. An HDAC6-decreasing agent according to claim 39, an HSP90 inhibitor according to claim 40 or an LC3-decreasing agent according to claim 41 for use in treating a disease or other condition in a cell or patient of interest, wherein the treating comprises administering the HDAC6-decreasing agent, HSP90 inhibitor or the LC3-decreasing agent simultaneously, separately or sequentially with a drug, and wherein the cell or patient of interest has high levels of HR23B.

43. A product comprising a drug in combination with one, two, three or all four of i) an HR23B-increasing agent, ii) an HDAC6-decreasing agent, iii) an HSP90 inhibitor, and iv) an LC3-decreasing agent in the treatment of a disease of other condition in a cell or patient of interest, wherein the drug in combination with one, two, three or all four of i) an HR23B-increasing agent, ii) an HDAC6-decreasing agent, iii) an HSP90 inhibitor, and iv) a LC3-decreasing agent are for simultaneous, separate or sequential administration

44. A method, biomarker, drug, HR23B-increasing agent, HDAC6-decreasing agent, HSP90 inhibitor, LC3-decreasing agent or product according to any one of the preceding claims, wherein the disease or other condition is cancer.

45. A method to optimise the dosage of a drug comprising administering the drug to a cell or patient of interest, monitoring whether an apoptotic state or an autophagy state ensues, and administering a further dose of drug which has been adjusted if necessary, or alternatively not administering a further dose of the drug.

46. A method of claim 45, which comprises monitoring a change in level of the HR23B biomarker optionally in combination with the level of one or more of the LC3, HDAC6 and HSP90 biomarkers following drug treatment and correlating this with an apoptotic or autophagy state.

47. A method, biomarker, kit, drug, inhibitor, agent or product according to any one of the preceding claims, wherein the drug is an anti-cancer drug.

48. A method, biomarker, kit, drug, inhibitor, agent or product according to any one of the preceding claims, wherein the drug is an HDAC inhibitor.

49. A method according to claim 1 or claim 2, which further comprises determining the level of HDAC6 in the cell or patient.

50. A method according to claim 1, claim 2 or claim 49, which further comprises determining the level of HSP90 in the cell or patient.


SEQUENCE LISTING (20 sequences)

SEQ ID NO: 1 - DNA, 30 bp, Artificial Sequence
  synthetic primer
ctactagcggccgcaatgacctcaacacgc

SEQ ID NO: 2 - DNA, 29 bp, Artificial Sequence
  synthetic primer
tagtaggcggccgcttatttatcatcatc

SEQ ID NO: 3 - DNA, 34 bp, Artificial Sequence
  synthetic primer
gtggtggaattcatgcaggtcaccctgaagaccc

SEQ ID NO: 4 - DNA, 36 bp, Artificial Sequence
  synthetic primer
tctagactcgagatcttcatcaaagttctgctgtag

SEQ ID NO: 5 - DNA, 34 bp, Artificial Sequence; identical to SEQ ID NO: 3
  synthetic primer

SEQ ID NO: 6 - DNA, 33 bp, Artificial Sequence
  synthetic primer
tctagactcgagagactgacccgtcacaagtgc

SEQ ID NO: 7 - DNA, 37 bp, Artificial Sequence
  synthetic primer
gtggtggaattcatggcagtgtccacaccagcaccag

SEQ ID NO: 8 - DNA, 36 bp, Artificial Sequence; identical to SEQ ID NO: 4
  synthetic primer

SEQ ID NO: 9 - RNA, 19 nt, Artificial Sequence
  synthetic siRNA
gaugcaacgagugcacuug

SEQ ID NO: 10 - DNA, 3648 bp, Homo sapiens
atgacctcaaccggccaggattccaccacaaccaggcagcgaagaagtaggcagaacccccagtcgccccctcaggactccagtgtcacttcgaagcgaaatattaaaaagggagccgttccccgctctatccccaatctagcggaggtaaagaagaaaggcaaaatgaagaagctcggccaagcaatggaagaagacctaatcgtgggactgcaagggatggatctgaaccttgaggctgaagcactggctggcactggcttggtgttggatgagcagttaaatgaattccattgcctctgggatgacagcttcccggaaggccctgagcggctccatgccatcaaggagcaactgatccaggagggcctcctagatcgctgcgtgtcctttcaggcccggtttgctgaaaaggaagagctgatgttggttcacagcctagaatatattgatctgatggaaacaacccagtacatgaatgagggagaactccgtgtcctagcagacacctacgactcagtttatctgcatccgaactcatactcctgtgcctgcctggcctcaggctctgtcctcaggctggtggatgcggtcctgggggctgagatccggaatggcatggccatcattaggcctcctggacatcacgcccagcacagtcttatggatggctattgcatgttcaaccacgtggctgtggcagcccgctatgctcaacagaaacaccgcatccggagggtccttatcgtagattgggatgtgcaccacggtcaaggaacacagttcaccttcgaccaggaccccagtgtcctctatttctccatccaccgctacgagcagggtaggttctggccccacctgaaggcctctaactggtccaccacaggtttcggccaaggccaaggatataccatcaatgtgccttggaaccaggtggggatgcgggatgctgactacattgctgctttcctgcacgtcctgctgccagtcgccctcgagttccagcctcagctggtcctggtggctgctggatttgatgccctgcaaggggaccccaagggtgagatggccgccactccggcagggttcgcccagctaacccacctgctcatgggtctggcaggaggcaagctgatcctgtctctggagggtggctacaacctccgcgccctggctgaaggcgtcagtgcttcgctccacacccttctgggagacccttgccccatgctggagtcacctggtgccccctgccggagtgcccaggcttcagtttcctgtgctctggaagcccttgagcccttctgggaggttcttgtgagatcaactgagaccgtggagagggacaacatggaggaggacaatgtagaggagagcgaggaggaaggaccctgggagccccctgtgctcccaatcctgacatggccagtgctacagtctcgcacagggctggtctatgaccaaaatatgatgaatcactgcaacttgtgggacagccaccaccctgaggtaccccagcgcatcttgcggatcatgtgccgtctggaggagctgggccttgccgggcgctgcctcaccctgacaccgcgccctgccacagaggctgagctgctcacctgtcacagtgctgagtacgtgggtcatctccgggccacagagaaaatgaaaacccgggagctgcaccgtgagagttccaactttgactccatctatatctgccccagtaccttcgcctgtgcacagcttgccactggcgctgcctgccgcctggtggaggctgtgctctcaggagaggttctgaatggtgctgctgtggtgcgtcccccaggacaccacgcagagcaggatgcagcttgcggtttttgctttttcaactctgtggctgtggctgctcgccatgcccagactatcagtgggcatgccctacggatcctgattgtggattgggatgtccaccacggtaatggaactcagcacatgtttgaggatgaccccagtgtgctatatgtgtccctgcaccgctatgatcatggcaccttcttccccatgggggatgagggtgccagcagccagatcggccgggctgcgggcacaggcttcaccgtcaacgtggcatggaacgggccccgcatgggtgatgctgactacctagctgcctggcatcgcctggtgcttcccattgcctacgagtttaacccagaactggtgctggtctcagctggctttgatgctgcacggggggatccgctggggggctgccaggtgtcacctgagggttatgcccacctcacccacctgctgatgggccttgccagtggccgcattatccttatcctagagggtggctataacctgacatccatctcagagtccatggctgcctgcactcgctccctccttggagacccaccacccctgctgaccctgccacggcccccactatcaggggccctggcctcaatcactgagaccatccaagtccatcgcagatactggcgcagcttacgggtcatgaaggtagaagacagagaaggaccctccagttctaagttggtcaccaagaaggcaccccaaccagccaaacctaggttagctgagcggatgaccacacgagaaaagaaggttctggaagcaggcatggggaaagtcacctcggcatcatttggggaagagtccactccaggccagactaactcagagacagctgtggtggccctcactcaggaccagccctcagaggcagccacagggggagccactctggcccagaccatttctgaggcagccattgggggagccatgctgggccagaccacctcagaggaggctgtcgggggagccactccggaccagaccacctcagaggagactgtgggaggagccattctggaccagaccacctcagaggatgctgttgggggagccacgctgggccagactacctcagaggaggctgtaggaggagctacactggcccagaccatctcggaggcagccatggagggagccacactggaccagactacgtcagaggaggctccagggggcaccgagctgatccaaactcctctagcctcgagcacagaccaccagacccccccaacctcacctgtgcagggaactacaccccagatatctcccagtacactgattgggagtctcaggaccttggagctaggcagcgaatctcagggggcctcagaatctcaggccccaggagaggagaacctactaggagaggcagctggaggtcaggacatggctgattcgatgctgatgcagggatctaggggcctcactgatcaggccatattttatgctgtgacaccactgccctggtgtccccatttggtggcagtatgccccatacctgcagcaggcctagacgtgacccaaccttgtggggactgtggaacaatccaagagaattgggtgtgtctctcttgctatcaggtctactgtggtcgttacatcaatggccacatgctccaacaccatggaaattctggacacccgctggtcctcagctacatcgacctgtcagcctggtgttactactgtcaggcctatgtccaccaccaggctctcctagatgtgaagaacatcgcccaccagaacaagtttggggaggatatgccccacccacactaa

SEQ ID NO: 11 - protein, 1215 aa, Homo sapiens
MTSTGQDSTTTRQRRSRQNPQSPPQDSSVTSKRNIKKGAVPRSIPNLAEVKKKGKMKKLGQAMEEDLIVGLQGMDLNLEAEALAGTGLVLDEQLNEFHCLWDDSFPEGPERLHAIKEQLIQEGLLDRCVSFQARFAEKEELMLVHSLEYIDLMETTQYMNEGELRVLADTYDSVYLHPNSYSCACLASGSVLRLVDAVLGAEIRNGMAIIRPPGHHAQHSLMDGYCMFNHVAVAARYAQQKHRIRRVLIVDWDVHHGQGTQFTFDQDPSVLYFSIHRYEQGRFWPHLKASNWSTTGFGQGQGYTINVPWNQVGMRDADYIAAFLHVLLPVALEFQPQLVLVAAGFDALQGDPKGEMAATPAGFAQLTHLLMGLAGGKLILSLEGGYNLRALAEGVSASLHTLLGDPCPMLESPGAPCRSAQASVSCALEALEPFWEVLVRSTETVERDNMEEDNVEESEEEGPWEPPVLPILTWPVLQSRTGLVYDQNMMNHCNLWDSHHPEVPQRILRIMCRLEELGLAGRCLTLTPRPATEAELLTCHSAEYVGHLRATEKMKTRELHRESSNFDSIYICPSTFACAQLATGAACRLVEAVLSGEVLNGAAVVRPPGHHAEQDAACGFCFFNSVAVAARHAQTISGHALRILIVDWDVHHGNGTQHMFEDDPSVLYVSLHRYDHGTFFPMGDEGASSQIGRAAGTGFTVNVAWNGPRMGDADYLAAWHRLVLPIAYEFNPELVLVSAGFDAARGDPLGGCQVSPEGYAHLTHLLMGLASGRIILILEGGYNLTSISESMAACTRSLLGDPPPLLTLPRPPLSGALASITETIQVHRRYWRSLRVMKVEDREGPSSSKLVTKKAPQPAKPRLAERMTTREKKVLEAGMGKVTSASFGEESTPGQTNSETAVVALTQDQPSEAATGGATLAQTISEAAIGGAMLGQTTSEEAVGGATPDQTTSEETVGGAILDQTTSEDAVGGATLGQTTSEEAVGGATLAQTTSEAAMEGATLDQTTSEEAPGGTELIQTPLASSTDHQTPPTSPVQGTTPQISPSTLIGSLRTLELGSESQGASESQAPGEENLLGEAAGGQDMADSMLMQGSRGLTDQAIFYAVTPLPWCPHLVAVCPIPAAGLDVTQPCGDCGTIQENWVCLSCYQVYCGRYINGHMLQHHGNSGHPLVLSYIDLSAWCYYCQAYVHHQALLDVKNIAHQNKFGEDMPHPH

SEQ ID NO: 12 - DNA, 2912 bp, Homo sapiens
cagttgcttcagcgtcccggtgtggctgtgccgttggtcctgtgcggtcacttagccaagatgcctgaggaaacccagacccaagaccaaccgatggaggaggaggaggttgagacgttcgcctttcaggcagaaattgcccagttgatgtcattgatcatcaatactttctactcgaacaaagagatctttctgagagagctcatttcaaattcatcagatgcattggacaaaatccggtatgaaactttgacagatcccagtaaattagactctgggaaagagctgcatattaaccttataccgaacaaacaagatcgaactctcactattgtggatactggaattggaatgaccaaggctgacttgatcaataaccttggtactatcgccaagtctgggaccaaagcgttcatggaagctttgcaggctggtgcagatatctctatgattggccagttcggtgttggtttttattctgcttatttggttgctgagaaagtaactgtgatcaccaaacataacgatgatgagcagtacgcttgggagtcctcagcagggggatcattcacagtgaggacagacacaggtgaacctatgggtcgtggaacaaaagttatcctacacctgaaagaagaccaaactgagtacttggaggaacgaagaataaaggagattgtgaagaaacattctcagtttattggatatcccattactctttttgtggagaaggaacgtgataaagaagtaagcgatgatgaggctgaagaaaaggaagacaaagaagaagaaaaagaaaaagaagagaaagagtcggaagacaaacctgaaattgaagatgttggttctgatgaggaagaagaaaagaaggatggtgacaagaagaagaagaagaagattaaggaaaagtacatcgatcaagaagagctcaacaaaacaaagcccatctggaccagaaatcccgacgatattactaatgaggagtacggagaattctataagagcttgaccaatgactgggaagatcacttggcagtgaagcatttttcagttgaaggacagttggaattcagagcccttctatttgtcccacgacgtgctccttttgatctgtttgaaaacagaaagaaaaagaacaatatcaaattgtatgtacgcagagttttcatcatggataactgtgaggagctaatccctgaatatctgaacttcattagaggggtggtagactcggaggatctccctctaaacatatcccgtgagatgttgcaacaaagcaaaattttgaaagttatcaggaagaatttggtcaaaaaatgcttagaactctttactgaactggcggaagataaagagaactacaagaaattctatgagcagttctctaaaaacataaagcttggaatacacgaagactctcaaaatcggaagaagctttcagagctgttaaggtactacacatctgcctctggtgatgagatggtttctctcaaggactactgcaccagaatgaaggagaaccagaaacatatctattatatcacaggtgagaccaaggaccaggtagctaactcagcctttgtggaacgtcttcggaaacatggcttagaagtgatctatatgattgagcccattgatgagtactgtgtccaacagctgaaggaatttgaggggaagactttagtgtcagtcaccaaagaaggcctggaacttccagaggatgaagaagagaaaaagaagcaggaagagaaaaaaacaaagtttgagaacctctgcaaaatcatgaaagacatattggagaaaaaagttgaaaaggtggttgtgtcaaaccgattggtgacatctccatgctgtattgtcacaagcacatatggctggacagcaaacatggagagaatcatgaaagctcaagccctaagagacaactcaacaatgggttacatggcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtattgatgaagatgaccctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggttctttcacagatggggtaacgtgcactgtaagacgtatgtaacatgatgttaactttgtgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaatactcgagcccagtcttgtggatggaaatgtagtgctcgagtcacattctgcttaaagttgtaacaaatacagatgagt

SEQ ID NO: 13 - protein, 732 aa, Homo sapiens
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 14 - DNA, 2543 bp, Homo Sapiens
ctccggcgcagtgttgggactgtctgggtatcggaaagcaagcctacgttgctcactattacgtataatccttttcttttcaagatgcctgaggaagtgcaccatggagaggaggaggtggagacttttgcctttcaggcagaaattgcccaactcatgtccctcatcatcaataccttctattccaacaaggagattttccttcgggagttgatctctaatgcttctgatgccttggacaagattcgctatgagagcctgacagacccttcgaagttggacagtggtaaagagctgaaaattgacatcatccccaaccctcaggaacgtaccctgactttggtagacacaggcattggcatgaccaaagctgatctcataaataatttgggaaccattgccaagtctggtactaaagcattcatggaggctcttcaggctggtgcagacatctccatgattgggcagtttggtgttggcttttattctgcctacttggtggcagagaaagtggttgtgatcagaaagcacaacgatgatgaacagtatgcttgggagtcttctgctggaggttccttcactgtgcgtgctgaccatggtgagcccattggcatgggtaccaaagtgatcctccatcttaaagaagatcagacagagtacctagaagagaggcgggtcaaagaagtagtgaagaagcattctcagttcataggctatcccatcaccctttatttggagaaggaacgagagaaggaaattagtgatgatgaggcagaggaagagaaaggtgagaaagaagaggaagataaagatgatgaagaaaagcccaagatcgaagatgtgggttcagatgaggaggatgacagcggtaaggataagaagaagaaaactaagaagatcaaagagaaatacattgatcaggaagaactaaacaagaccaagcctatttggaccagaaaccctgatgacatcacccaagaggagtatggagaattctacaagagcctcactaatgactgggaagaccacttggcagtcaagcacttttctgtagaaggtcagttggaattcagggcattgctatttattcctcgtcgggctccctttgacctttttgagaacaagaagaaaaagaacaacatcaaactctatgtccgccgtgtgttcatcatggacagctgtgatgagttgataccagagtatctcaattttatccgtggtgtggttgactctgaggatctgcccctgaacatctcccgagaaatgctccagcagagcaaaatcttgaaagtcattcgcaaaaacattgttaagaagtgccttgagctcttctctgagctggcagaagacaaggagaattacaagaaattctatgaggcattctctaaaaatctcaagcttggaatccacgaagactccactaaccgccgccgcctgtctgagctgctgcgctatcatacctcccagtctggagatgagatgacatctctgtcagagtatgtttctcgcatgaaggagacacagaagtccatctattacatcactggtgagagcaaagagcaggtggccaactcagcttttgtggagcgagtgcggaaacggggcttcgaggtggtatatatgaccgagcccattgacgagtactgtgtgcagcagctcaaggaatttgatgggaagagcctggtctcagttaccaaggagggtctggagctgcctgaggatgaggaggagaagaagaagatggaagagagcaaggcaaagtttgagaacctctgcaagctcatgaaagaaatcttagataagaaggttgagaaggtgacaatctccaatagacttgtgtcttcaccttgctgcattgtgaccagcacctacggctggacagccaatatggagcggatcatgaaagcccaggcacttcgggacaactccaccatgggctatatgatggccaaaaagcacctggagatcaaccctgaccaccccattgtggagacgctgcggcagaaggctgaggccgacaagaatgataaggcagttaaggacctggtggtgctgctgtttgaaaccgccctgctatcttctggcttttcccttgaggatccccagacccactccaaccgcatctatcgcatgatcaagctaggtctaggtattgatgaagatgaagtggcagcagaggaacccaatgctgcagttcctgatgagatcccccctctcgagggcgatgaggatgcgtctcgcatggaagaagtcgattaggttaggagttcatagttggaaaacttgtgcccttgtatagtgtccccatgggctcccactgcagcctcgagtgcccctgtcccacctggctccccctgctggtgtctagtgtttttttccctctcctgtccttgtgttgaaggcagtaaactaagggtgtcaagccccattccctctctactcttgacagcaggattggatgttgtgtattgtggtttattttattttcttcattttgttctgaaattaaagtatgcaaaataaagaatatgccgtttttatac

SEQ ID NO: 15 - protein, 724 aa, Homo sapiens
MPEEVHHGEEEVETFAFQAEIAQLMSLIINTFYSNKEIFLRELISNASDALDKIRYESLTDPSKLDSGKELKIDIIPNPQERTLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKDDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDELIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKAKFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALLSSGFSLEDPQTHSNRIYRMIKLGLGIDEDEVAAEEPNAAVPDEIPPLEGDEDASRMEEVD

SEQ ID NO: 16 - protein, 409 aa, Homo sapiens
MQVTLKTLQQQTFKIDIDPEETVKALKEKIESEKGKDAFPVAGQKLIYAGKILNDDTALKEYKIDEKNFVVVMVTKPKAVSTPAPATTQQSAPASTTAVTSSTTTTVAQAPTPVPALAPTSTPASITPASATASSEPAPASAAKQEKPAEKPAETPVATSPTATDSTSGDSSRSNLFEDATSALVTGQSYENMVTEIMSMGYEREQVIAALRASFNNPDRAVEYLLMGIPGDRESQAVVDPPQAASTGAPQSSAVAAAAATTTATTTTTSSGGHPLEFLRNQPQFQQMRQIIQQNPSLLPALLQQIGRENPQLLQQISQHQEHFIQMLNEPVQEAGGQGGGGGGGSGGIAEAGSGHMNYIQVTPQEKEAIERLKALGFPEGLVIQAYFACEKNENLAANFLLQQNFDED

SEQ ID NO: 17 - protein, 121 aa, Artificial Sequence
  Homo sapiens
MPSDRPFKQRRSFADRCKEVQQIRDQHPSKIPVIIERYKGEKQLPVLDKTKFLVPDHVNMSELVKIIRRRLQLNPTQAFFLLVNQHSMVSVSTPIADIYEQEKDEDGFLYMVYASQETFGF

SEQ ID NO: 18 - protein, 14 aa, Homo sapiens
DPEETVKALKEKIE

SEQ ID NO: 19 - protein, 14 aa, Homo sapiens
PAPASAAKQEKPAE

SEQ ID NO: 20 - protein, 14 aa, Homo sapiens
VATSPTATDSTSGD